(19) United States
(12) Patent Application Publication
(10) Pub. No.: US 2009/0252678 A1
(43) Pub. Date: Oct. 8, 2009

(54) COX-2-TARGETED IMAGING AGENTS

(75) Inventors: Lawrence J. Marnett, Nashville, TN (US); Sergei Timofeevski, Carlsbad, CA (US); Daniel Prudhomme, Nashville, TN (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: Vanderbilt University, Nashville, TN (US)

(21) Appl. No.: 12/423,358

(22) Filed: Apr. 14, 2009

Related U.S. Application Data
(62) Division of application No. 10/877,303, filed on Jun. 25, 2004, now abandoned.
(60) Provisional application No. 60/482,422, filed on Jun. 25, 2003.

(51) Int. Cl. A61K 51/04

(52) U.S. Cl. 424/1.81; 424/1.65

(57) ABSTRACT

The presently disclosed subject matter provides a method for synthesizing a radiological imaging agent by reacting a COX-2-selective ligand with a compound comprising a detectable group, wherein the COX-2-selective ligand is a derivative of a non-steroidal anti-inflammatory drug (NSAID) comprising an ester moiety or a secondary amide moiety. Also provided are compositions that are synthesized using the method, as well as methods of using the compositions of the presently disclosed subject matter.

### CROSS REFERENCE TO RELATED APPLICATIONS

This application is a divisional of U.S. patent application Ser. No. 10/877,303, filed Jun. 25, 2004 (now abandoned), which itself is based on and claims priority to U.S. Provisional Application Ser. No. 60/482,422, filed Jun. 25, 2003. The disclosure of each of these applications is incorporated herein by reference in its entirety.

### GRANT STATEMENT

This work was supported by grant CA85283 from the United States National Institutes of Health. Accordingly, the United States Government has certain rights in the presently disclosed subject matter.

### TECHNICAL FIELD

The presently disclosed subject matter generally relates to imaging agents that comprise COX-2-selective ligands. More particularly, the presently disclosed subject matter relates to derivatives of non-steroidal anti-inflammatory drugs that exhibit binding to cyclooxygenase-2 (COX-2) and that comprise functional groups allowing them to be used as radiological imaging agents.

### Table of Abbreviations

¹¹C—carbon-11
¹⁸F—fluorine-18
ACN—acetonitrile
ApC^{Min-}—a mouse strain that is highly susceptible to the formation of spontaneous intestinal adenomas
APHS—o-(acetoxyphenyl)hept-2-ynyl sulfide
At—astatine
BOC—tert-butoxycarbonyl
(BOC)₂O—Di-tert-butyl dicarbonate
Br—bromine
Cl—chlorine
COX-1—cyclooxygenase 1
COX-2—cyclooxygenase 2
CID—collision-induced dissociation
CT—computed tomography
DIPEA—diisopropylethylamine
DMAP—4-(dimethylamino)pyridine
DMF—dimethylformamide
DMSO—dimethyl sulfoxide
DOTA—tetraazacyclododecyltetraacetic acid
DTPA—diethylenetriamine pentaacetate
ED₅₀—effective dose 50
EDCl—1-ethyl-3-(3′-dimethylaminopropyl)carbodiimide
ELISA—enzyme-linked immunosorbent assay
ESI—electrospray ionization
Et—ethyl
ETYA—5,8,11,14-eicosatetraynoic acid
F—fluorine
FAP—familial adenomatous polyposis
F-APHS—fluoroacetyl derivative of o-(acetoxyphenyl)hept-2-ynyl sulfide
FDA—U.S. Food and Drug Administration
HCl_{(g)}—HCl gas
HOBt—N-hydroxybenzotriazole
I—iodine
IC₅₀—concentration that inhibits by 50%
INDO—indomethacin
keV—kiloelectron volts
k_{inact}—rate constant for inactivation
Kᵢ—inhibition constant
LAH—lithium aluminum hydride
LPS—lipopolysaccharide
MPM—mouse resident peritoneal macrophages
NIR—near infrared
NIH—National Institutes of Health
NMe₂—N,N-dimethyl
NMe₃—N,N,N-trimethyl
NSAIDs—non-steroidal anti-inflammatory drugs
PET—positron emission tomography
PG—prostaglandin
PGD₂—prostaglandin D₂
PGE₂—prostaglandin E₂
PGG₂—prostaglandin G₂
PGH₂—prostaglandin H₂
SPECT—single photon emission computed tomography
TEA—triethylamine
THF—tetrahydrofuran
TLC—thin layer chromatography
Ts-Cl—tosyl chloride
TXA₂—thromboxane A₂
TXB₂—thromboxane B₂

### BACKGROUND

A limitation of current diagnostic imaging methods is that it is often not possible to deliver the imaging agent specifically to the tissue or cell type that one wishes to image. In the case of target tissue imaging, what is needed is an agent that is specific for the target tissue, yet does not bind appreciably to surrounding non-target cells. Particularly desirable as imaging agents are compounds that can be used with non-invasive imaging techniques such as positron emission tomography (PET) and others.

In the area of diagnostic imaging of cancer, current methods for tumor-specific imaging are hindered by imaging agents that also accumulate in normal tissues. Additionally, a lack of targeting ligands that are capable of binding to multiple tumor types necessitates the synthesis of a wide range of agents in order to image different tumor types. Ideally, a targeting molecule should display specific targeting in the absence of substantial binding to normal tissues, and a capacity for targeting to a variety of tumor types and stages. Finally, early diagnosis of neoplastic changes can result in more effective treatment of cancer. Thus, there exists a long-felt need in the art for methods to achieve delivery of imaging agents to tumors early in the course of tumorigenesis.

Cyclooxygenase (COX) activity originates from two distinct and independently regulated enzymes, termed COX-1 and COX-2 (see DeWift and Smith, 1988; Yokoyama and Tanabe, 1989; Hla and Neilson, 1992). COX-1 is a constitutive isoform and is mainly responsible for the synthesis of cytoprotective prostaglandin in the gastrointestinal tract and for the synthesis of thromboxane, which triggers aggregation of blood platelets (Allison et al., 1992). COX-2, on the other hand, is inducible and short-lived. Its expression is stimulated in response to endotoxins, cytokines, and mitogens (Kujubu et al., 1991; Lee et al., 1992; O'Sullivan et al., 1993).

Cyclooxygenase-2 (COX-2) catalyzes the committed step in the biosynthesis of prostaglandins, thromboxane, and prostacyclin (Smith et al., 2000). COX-2 is not expressed in most normal tissues, but is present in inflammatory lesions and tumors (Fu et al., 1990; Eberhart et al., 1994). Studies by Eberhart et al. and Kargman et al. by first demonstrated that COX-2 mRNA and protein are expressed in tumor cells from colon cancer patients but not in surrounding normal tissue (Eberhart et al., 1994; Kargman et al., 1995). COX-2 expression appears to be an early event in colon tumorigenesis because it is detectable in colon polyps (Eberhart et al., 1994). Approximately 55% of polyps demonstrate COX-2 expression compared to approximately 85% of colon adenocarcinomas. The concept that COX-2 is expressed in malignant tumors and their precursor lesions has been extended to a broader range of solid tumors including those of the esophagus (Kandil et al., 2001), bladder (Ristimaki et al., 2001), breast (Ristimaki et al., 2002), pancreas (Tucker et al., 1999), lung (Soslow et al., 2000), and melanoma (Denkert et al., 2001).

The expression of COX-2 in tumors appears to have functional consequences. Prostaglandins have been demonstrated to stimulate cell proliferation (Marnett, 1992), inhibit apoptosis (Tsujii and DuBois, 1995), increase cell motility (Sheng et al., 2001), and enhance angiogenesis in animal models (Daniel et al., 1999; Masferrer et al., 2000). COX-2 expression is dramatically elevated in rodent models of colon cancer and crossing COX-2 knockout mice into the APC^{Min-} background (a mouse strain that is highly susceptible to the formation of spontaneous intestinal adenomas) reduces the number of intestinal tumors by ˜85% compared to APC^{Min-} controls (DuBois et al., 1996; Oshima et al., 1996). COX-2 expression is detected in breast cancers from the subset of patients exhibiting Her-2/neu overexpression. Overexpression of COX-2 specifically targeted to the breast of multiparous rodents induces breast cancer. These findings suggest that COX-2 contributes to tumor progression so that its expression in tumor tissue plays an important functional role. In fact, high COX-2 expression in tumors is associated with poor clinical outcome (Tucker et al., 1999; Denkert et al., 2001; Kandil et al., 2001; Ristimaki et al., 2002). Consequently, several clinical trials have been initiated to evaluate the potential of COX-2 inhibitors as chemopreventive agents and adjuvants to chemotherapy.

COX-2 is a molecular target for the anti-inflammatory, analgesic, and antipyretic effects of non-steroidal anti-inflammatory drugs (NSAIDs), particularly the recently developed COX-2-selective inhibitors, celecoxib (sold under the trade name CELEBREX® by Pfizer Inc. of New York, N.Y., United States of America) and rofecoxib (sold under the trade name VIOXX® by Merck and Co., Inc. of Whitehouse Station, N.J., United States of America). See also Vane and Botting, 1996. NSAIDs exhibit varying selectivity for COX-2 and COX-1 but, in general, few of them display high selectivity for COX-2 (Meade et al., 1993). NSAIDs possess cancer chemopreventive activity, while COX-selective drugs retard the growth of human tumor xenografts in nude mice and induce polyp regression in individuals with familial polyposis (Sheng et al., 1997; Kawamori et al., 1998; Steinbach et al., 2000). These activities have been attributed to these drugs' ability to inhibit COX-2.

### SUMMARY

A method for synthesizing a radiological imaging agent is disclosed. In some embodiments, the method comprises reacting a COX-2-selective ligand with a compound comprising a detectable group, wherein the COX-2-selective ligand is a derivative of a non-steroidal anti-inflammatory drug (NSAID) comprising an ester moiety or a secondary amide moiety. In some embodiments, a carboxylic acid group of the NSAID has been derivatized to an ester or a secondary amine.

In some embodiments, the NSAID is selected from the group consisting of fenamic acids, indoles, phenylalkanoic acids, phenylacetic acids, pharmaceutically acceptable salts thereof, and combinations thereof. In some embodiments, the NSAID is selected from the group consisting of aspirin, o-(acetoxyphenyl)hept-2-ynyl sulfide (APHS), indomethacin, 6-methoxy-α-methyl-2-naphthylacetic acid, meclofenamic acid, 5,8,11,14-eicosatetraynoic acid (ETYA), diclofenac, flufenamic acid, niflumic acid, mefenamic acid, sulindac, tolmetin, suprofen, ketorolac, flurbiprofen, ibuprofen, aceloferac, alcofenac, amfenac, benoxaprofen, bromfenac, carprofen, clidanac, diflunisal, efenamic acid, etodolic acid, fenbufen, fenclofenac, fenclorac, fenoprofen, fleclozic acid, indoprofen, isofezolac, ketoprofen, loxoprofen, meclofenamate, naproxen, orpanoxin, pirprofen, pranoprofen, tolfenamic acid, zaltoprofen, zomepirac, and pharmaceutically acceptable salts thereof, and combinations thereof. In some embodiments, the NSAID is selected from the group consisting of aspirin, o-(acetoxyphenyl)hept-2-ynyl sulfide (APHS), indomethacin, meclofenamic acid, 5,8,11,14-eicosatetraynoic acid (ETYA), ketorolac, and pharmaceutically acceptable salts thereof, and combinations thereof.

In some embodiments, the secondary amide derivative is selected from the group consisting of indomethacin-N-methyl amide, indomethacin-N-ethan-2-ol amide, indomethacin-N-octyl amide, indomethacin-N-nonyl amide, indomethacin-N-(2-methylbenzyl)amide, indomethacin-N-(4-methylbenzyl)amide, indomethacin-N—[(R)-α,4-dimethylbenzyl]amide, indomethacin-N—((S)-α,4-dimethylbenzyl)amide, indomethacin-N-(2-phenethyl)amide, indomethacin-N-(4-fluorophenyl)amide, indomethacin-N-(4-chlorophenyl)amide, indomethacin-N-(4-acetamidophenyl)amide, indomethacin-N-(4-methylmercapto)phenyl amide, indomethacin-N-(3-methylmercaptophenyl)amide, indomethacin-N-(4-methoxyphenyl)amide, indomethacin-N-(3-ethoxyphenyl)amide, indomethacin-N-(3,4,5-trimethoxyphenyl)amide, indomethacin-N-(3-pyridyl)amide, indomethacin-N-5-[(2-chloro)pyridyl]amide, indomethacin-N-5-[(1-ethyl)pyrazolo]amide, indomethacin-N-(3-chloropropyl)amide, indomethacin-N-methoxycarbonylmethyl amide, indomethacin-N-2-(2-L-methoxycarbonylethyl)amide, indomethacin-N-2-(2-D-methoxycarbonylethyl)amide, indomethacin-N-(4-methoxycarbonylbenzoyl)amide, indomethacin-N-(4-methoxycarbonylmethylphenyl)amide, indomethacin-N-(2-pyrazinyl)amide, indomethacin-N-2-(4-methylthiazolyl)amide, indomethacin-N-(4-biphenyl)amide, and combinations thereof.

In some embodiments of the present method, the detectable group is selected from the group consisting of a halogen-containing moiety, a fluorescent moiety, a metal ion-chelating moiety, a dye, a radioisotope-containing moiety, and combinations thereof. In some embodiments, the halogen-containing moiety comprises a chloride atom, a fluorine atom, an iodine atom, a bromine atom, or a radioactive isotope thereof.

The presently disclosed subject matter also provides a method for imaging a target tissue in a subject. In some embodiments, the method comprises administering to the subject a radiological imaging agent under conditions sufficient for binding the radiological imaging agent to the target tissue, wherein the radiological imaging agent comprises a derivative of a non-steroidal anti-inflammatory drug (NSAID) comprising an ester moiety or a secondary amide moiety and further comprises a detectable group, and detecting the detectable group in the target tissue. In some embodiments of the method, a carboxyl group of the non-steroidal anti-inflammatory drug is derivatized to an ester or secondary amide.

In some embodiments, the target tissue is selected from the group consisting of an inflammatory lesion, a pre-neoplastic lesion, a tumor, a neoplastic cell, a pre-neoplastic cell, and a cancer cell. In some embodiments, the pre-neoplastic lesion is selected from the group consisting of a colon polyp and Barrett's esophagus. In some embodiments, the tumor is selected from the group consisting of a primary tumor, a metastasized tumor, and a carcinoma.

In some embodiments of the present method, the subject is a mammal. In some embodiments, the mammal is a human.

Various routes of administration of the imaging agent can be employed in the disclosed methods. In some embodiments, the administering is via a route selected from the group consisting of peroral, intravenous, intraperitoneal, inhalation, and intratumoral.

In some embodiments, the (NSAID) is selected from the group consisting of fenamic acids, indoles, phenylalkanoic acids, phenylacetic acids, pharmaceutically acceptable salts thereof, and combinations thereof. In some embodiments, the NSAID is selected from the group consisting of aspirin, o-(acetoxyphenyl)hept-2-ynyl sulfide (APHS), indomethacin, 6-methoxy-α-methyl-2-naphthylacetic acid, meclofenamic acid, 5,8,11,14-eicosatetraynoic acid (ETYA), diclofenac, flufenamic acid, niflumic acid, mefenamic acid, sulindac, tolmetin, suprofen, ketorolac, flurbiprofen, ibuprofen, aceloferac, alcofenac, amfenac, benoxaprofen, bromfenac, carprofen, clidanac, diflunisal, efenamic acid, etodolic acid, fenbufen, fenclofenac, fenclorac, fenoprofen, fleclozic acid, indoprofen, isofezolac, ketoprofen, loxoprofen, meclofenamate, naproxen, orpanoxin, pirprofen, pranoprofen, tolfenamic acid, zaltoprofen, zomepirac, and pharmaceutically acceptable salts thereof, and combinations thereof. In some embodiments, the NSAID is selected from the group consisting of aspirin, o-(acetoxyphenyl)hept-2-ynyl sulfide (APHS), indomethacin, meclofenamic acid, 5,8,11,14-eicosatetraynoic acid (ETYA), ketorolac, and pharmaceutically acceptable salts thereof, and combinations thereof.

The disclosed methods can employ radiological and/or optical imaging agents as disclosed herein. In some embodiments of the presently disclosed subject matter, the imaging agent comprises the following structure:

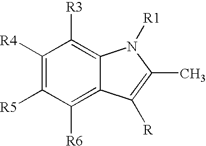

wherein
R is selected from the group consisting of

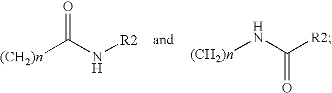

R1 is selected from the group consisting of a detectable group,

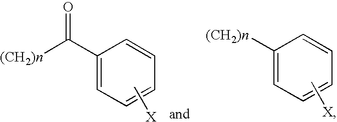

wherein X is a halogen or a radioactive isotope thereof at one or more positions of the aromatic ring;
R2 comprises a detectable group or a halo substituted aryl;
R3, R4, R5, and R6 are each independently selected from the group consisting of hydrogen; halo; C₁ to C₆ alkyl or branched alkyl; C₁ to C₆ alkoxy or branched alkoxy; benzyloxy; SCH₃; SOCH₃; SO₂CH₃; SO₂NH₂; and CONH₂;
n is 0-5 inclusive;
   and wherein at least one of R1 and R2 comprises a detectable group.

In some embodiments, the imaging agent comprises the following structure:

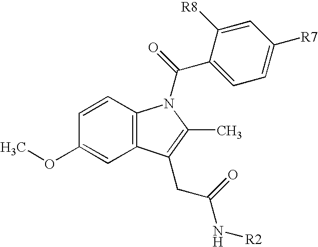

wherein R7 comprises a halogen and R8 is selected from the group consisting of hydrogen, a halogen, C₁-C₆ alkyl or branched alkyl, and C₁-C₆ aryl or branched aryl. In some embodiments, R3 is ¹⁸F.

In some embodiments of the imaging agent, R7 is Cl and R2 has the following structure:

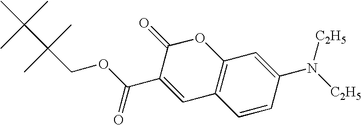

In some embodiments, R7 is Cl and R2 has the following structure:

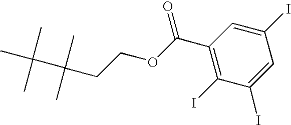

In some embodiments, R7 is Cl and R2 has the following structure:

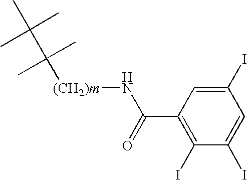

wherein m=an integer between 0 and 8, inclusive.

In some embodiments, R7 is Cl and R2 has the following structure:

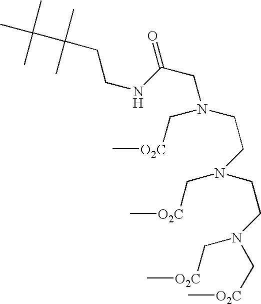

In some embodiments, R2 further comprises a coordinated metal ion. In some embodiments, the coordinated metal ion is selected from the group consisting of Gd³⁺, Eu³⁺, Fe³⁺, Mn²⁺, Yt³⁺, Dy³⁺, and Cr³⁺. In some embodiments, the coordinated metal ion is Gd³⁺ or Eu³⁺.

In some embodiments, R7 is Cl and R2 has the following structure:

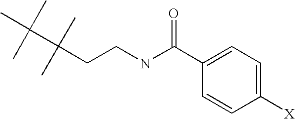

wherein X is a halogen or a radioactive isotope thereof. In some embodiments, X is ¹⁸F.

In some embodiments, R7 is Cl and R2 has the following structure:

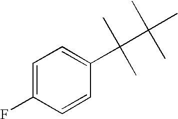

In some embodiments, R7 is Cl and R2 has the following structure:

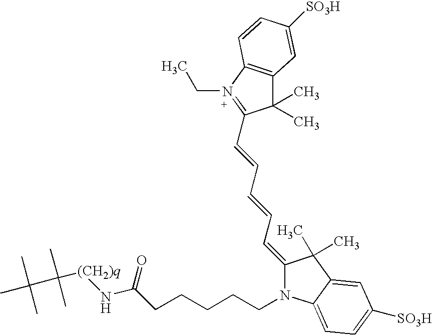

wherein q=an integer between 0 and 8, inclusive.

In some embodiments, the imaging agent comprises the following structure:

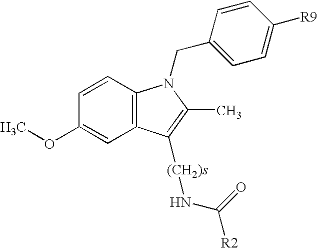

wherein R9 is a halogen, R2 is p-halobenzene, and s=14. In some embodiments, R9 is Br, s=2, and R2 is p-¹⁸F-benzene.

In some embodiments, the imaging agent comprises the following structure:

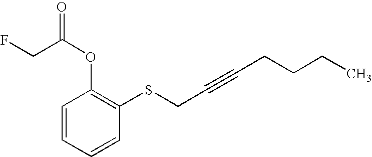

In some embodiments, the fluorine atom is ¹⁸F.

In some embodiments, the imaging agent comprises the following structure:

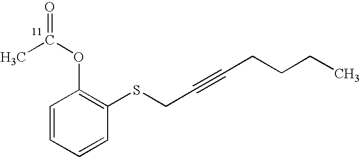

In some embodiments, the imaging agent comprises the following structure:

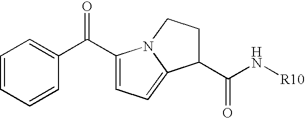

wherein R10 comprises a detectable group. In some embodiments of this aspect, R10 has the following structure:

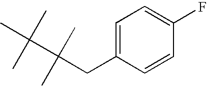

In some embodiments, the imaging agent comprises the following structure:

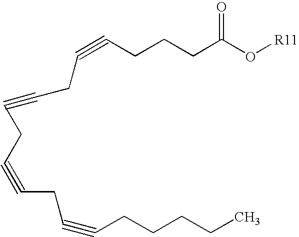

wherein R11 comprises a detectable group selected from the group consisting of a halogen-containing moiety, a fluorescent moiety, a metal ion-chelating moiety, a dye, a radioisotope-containing moiety, and combinations thereof.

In some embodiments, the imaging agent comprises the following structure:

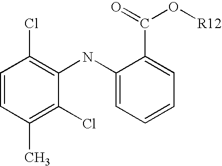

wherein R12 comprises a detectable group selected from the group consisting of a halogen-containing moiety, a fluorescent moiety, a metal ion-chelating moiety, a dye, a radioisotope-containing moiety, and combinations thereof.

According to the present disclosure, the imaging agent comprises a detectable group. In some embodiments, the detectable group is selected from the group consisting of a halogen-containing moiety, a fluorescent moiety, a metal ion-chelating moiety, a dye, a radioisotope-containing moiety, and combinations thereof. The detectable group can be detected using various radiological and/or optical detection methodologies. In some embodiments, the detecting is by positron emission tomography, near infrared luminescence, or monochromatic X-ray.

The presently disclosed subject matter also provides an imaging agent comprising a detectable group and an indomethacin derivative, wherein the agent is selected from the group consisting of a compound having one of the following structures:

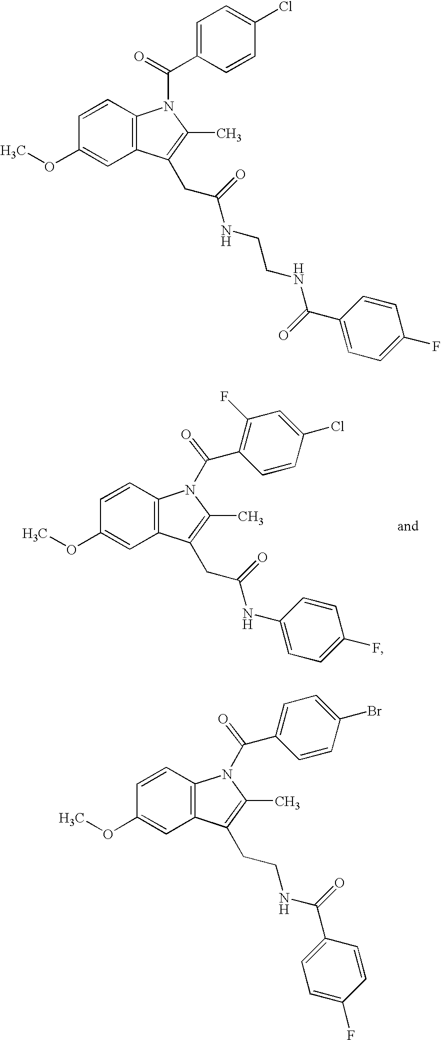

In some embodiments, the detectable group comprises ¹⁸F. In some embodiments, one or more fluorine atoms present in the structures listed above is ¹⁸F.

### BRIEF DESCRIPTION OF THE DRAWINGS

FIG. 1 depicts the general reaction catalyzed by cyclooxygenases by which arachidonic acid is converted to prostaglandin G₂ (PGG₂) and then to prostaglandin H₂ (PGH₂).

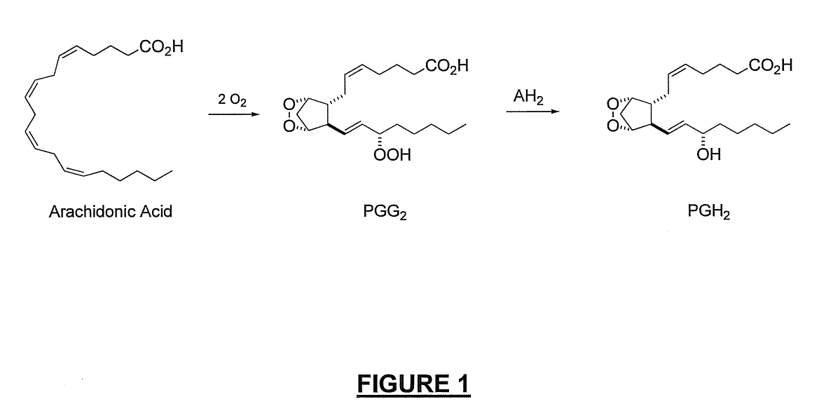

FIG. 2 depicts the conversion of aspirin to o-(acetoxyphenyl)hept-2-ynyl sulfide (APHS).

FIG. 3 depicts the conversion of indomethacin to COX-2-selective ligands Compounds 1 and 2.

FIG. 4 depicts Compound 3, a coumarin-derived ester of the ethanolamide of indomethacin.

FIG. 5 depicts the structures of 5,8,11,14-eicosatetraynoic acid (ETYA), meclofenamic acid, ketorolac, and indomethacin, four NSAIDs to which the disclosed conversion process has been successfully applied.

FIG. 6 depicts the structures of several indomethacin derivatives that bind to COX-2. None of the compounds shown inhibits COX-1 up to 66 μM.

FIG. 7 depicts the synthesis of Compounds 4 and 5, which are iodine-containing contrast agents. EDCl: 1-ethyl-3-(3′-dimethylaminopropyl)carbodiimide; DMAP: 4-(dimethylamino)pyridine.

FIG. 8 depicts the synthesis of two iodine-containing contrast agents tethered via amide linkages of varying length. EDCl: 1-ethyl-3-(3′-dimethylaminopropyl)carbodiimide; HOBt: N-Hydroxybenzotriazole; DMF: dimethylformamide.

FIG. 9 depicts an alternate synthesis scheme for the construction of iodine-containing contrast agents Compounds 8 and 10-12. EDCl: 1-ethyl-3-(3′-dimethylaminopropyl)carbodiimide; DMAP: 4-(dimethylamino)pyridine; TEA: triethylamine; DMF: dimethylformamide; INDO: indomethacin.

FIG. 10 depicts the synthesis of Compound 14, a heavy metal chelating agent tethered to indomethacin.

FIG. 11 depicts Compounds 16-18, which are indomethacin derivatives.

FIG. 12 depicts two alternative routes for the synthesis of ¹⁸F-APHS. Et: ethyl group, CH₂CH₃.

FIG. 13 depicts the synthesis of ¹¹C-APHS.

FIG. 14 depicts the synthesis of ¹⁸F-containing Compound 18. Also shown are the fluorinated ketorolac and diarylpyrazole derivatives, Compounds 19 and 20, respectively.

FIG. 15 depicts the synthesis of indomethacin-based dyes for NIR luminescence imaging.

FIG. 16 depicts a scheme for synthesizing indoyl amide derivatives of indomethacin, including a fluoro-standard, Compound 389, and a PET precursor, Compound 390. For each step, the components of each reaction are symbolized by an encircled lowercase letter. The components of each reaction are as follows: a: ammonium chloride, EDCl, HOBt, DIPEA, and DMF; b: LAH and THF; c: (BOC)₂O and DMF; d: NaH, bromobenzylbromide, and DMF; e: HCl_{(g)} and dichloromethane; f: 4-F—C₆H₄CO₂H, EDCl, HOBt, DIPEA, and DMF; g: 4-NO₂—C₆H₄CO₂H, EDCl, HOBt, DIPEA, and DMF; h: KRYPTOFIX_{2,2,2}®, ¹⁸F-KF, and ACN.

FIG. 17 depicts a scheme for synthesizing various diamide derivatives of indomethacin. For each step, the components of each reaction are symbolized by an encircled lowercase letter. The components of each reaction are as follows: a: N—BOC-ethylenediamine, EDCl, HOBt, DIPEA, and DMF; b: HCl_{(g)} and dichloromethane; c: EDCl, HOBt, DIPEA, and DMF (X═I, F, NO₂, OH, or NMe₂); d: CF₃SO₃CH₃ and dichloromethane; e: Ts-Cl and dichloromethane; f: KRYPTOFIX_{2,2,2}®, ¹⁸F-KF, and ACN.

FIG. 18 depicts a scheme for synthesizing amide derivatives of indomethacin. For each step, the components of each reaction are symbolized by an encircled lowercase letter. The components of each reaction are as follows: a: 10 N NaOH and DMF; b: 4-fluoroaniline, EDCl, HOBt, DMAP, and dichloromethane; c: NaH, 4-chloro-2-nitro-benzoyl chloride, and DMF; d: SOCl₂, pyridine, and DMF; e: NaH, 4-chloro-2-fluoro-benzoyl chloride, and DMF; f: KRYPTOFIX_{2,2,2}®, ¹⁸F-KF, and ACN.

FIG. 19 depicts a scheme for production of ¹⁸F and the exchange chemistry that can be used to radiolabel NSAID (for example, indomethacin) derivatives to create COX-2-targeted imaging agents. For each step, the components of each reaction are symbolized by an encircled lowercase letter. The components of each reaction are as follows: a: ₁₀KV bombardment; b: K₂CO₃; c: KRYPTOFIX_{2,2,2}®, DMSO, 85° C., with X═F, NO₂, I, OTs, or NMe₃⁺.

### DETAILED DESCRIPTION

The present subject matter will be now be described more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Unless otherwise defined, all technical and scientific terms used herein have the same meaning as commonly understood by one of ordinary skill in the art to which the presently disclosed subject matter belongs. All publications, patent applications, patents, and other references mentioned herein are incorporated by reference in their entirety.

Throughout the specification and claims, a given chemical formula or name shall encompass all optical and stereoisomers as well as racemic mixtures where such isomers and mixtures exist.

Throughout the specification, drawings, and claims, some chemical structures are depicted without including certain methyl groups and/or hydrogens. In the structures, solid lines represent bonds between two atoms, and unless otherwise indicated, between carbon atoms. Thus, bonds that have no atom specifically recited on one end and/or the other have a carbon atom at that and/or the other end. For example, a structure depicted as “—O—” represents C—O—C. Given that hydrogens are not explicitly placed in all structures, implicit hydrogens are understood to exist in the structures as necessary. Thus, a structure depicted as “—O” can represent H₃C—O, as appropriate given the valences of the particular atoms.

Additionally, throughout the specification, including the drawings and the claims, a bond that is depicted as such

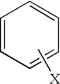

is intended to represent an aromatic ring in which one or more of the hydrogens is replaced by another moiety, such as a halogen or a radioactive isotope thereof. As used herein, this schematic representation also represents aromatic rings in which more than one hydrogen has been replaced. In those embodiments in which more than one hydrogen has been replaced, the schematic depiction is intended to represent any combination of different moieties (e.g. halogens and/or radioactive isotopes thereof) in any of the possible positions of the aromatic ring.

Following long-standing patent law convention, the terms “a” and “an” mean “one or more” when used in this application, including the claims.

### I. General Considerations

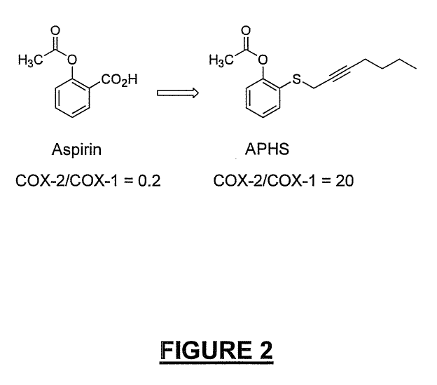

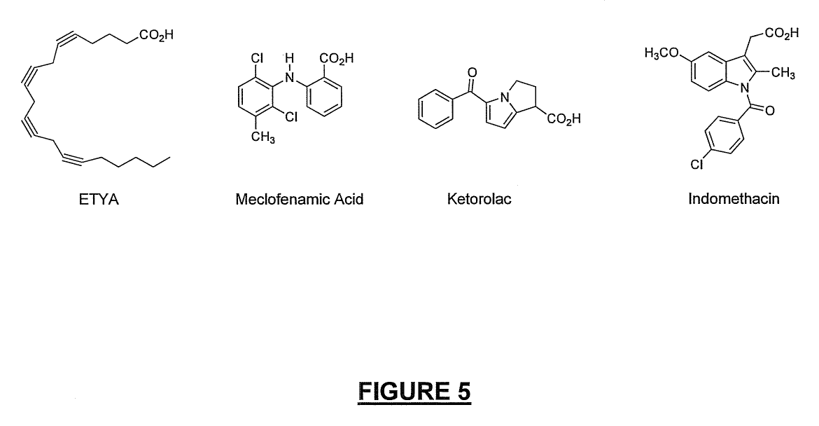

Novel approaches have recently been developed that allow the facile conversion of non-selective NSAIDs into highly selective COX-2 ligands (Kalgutkar et al., 1998a; Kalgutkar et al., 2000a). This is accomplished by conversion of the carboxylic acid functional group, common to most NSAIDs, to a derivative. In one strategy, aspirin, an NSAID that covalently modifies COX-1 and COX-2 by acetylation, was converted to an acetylating agent, o-(acetoxyphenyl)hept-2-ynyl sulfide (APHS), that is 100 times more selective for COX-2 than aspirin (Kalgutkar et al., 1998a; see also FIG. 2). Utilizing another strategy, it was discovered that several carboxylic acid-containing NSAIDs can be transformed into highly selective COX-2 inhibitors by converting them into neutral amide or ester derivatives (Kalgutkar et al., 2000b). This strategy has proven effective in the case of the NSAIDs 5,8,11,14-eicosatetraynoic acid (ETYA), meclofenamic acid, ketorolac, and indomethacin (FIG. 5). In the cases of ETYA, ketorolac, and meclofenamic acid, their amide derivatives exhibit selective COX-2 inhibitory activity. Several of the most potent inhibitors are haloalkyl or haloaryl amide derivatives, including the p-fluorobenzylamide of ketorolac (IC₅₀-COX-2=80 nM; IC₅₀-COX-1>65 μM) and the p-fluorophenylamide of indomethacin (IC₅₀-COX-2=52 nM; IC₅₀-COX-1>66 μM).

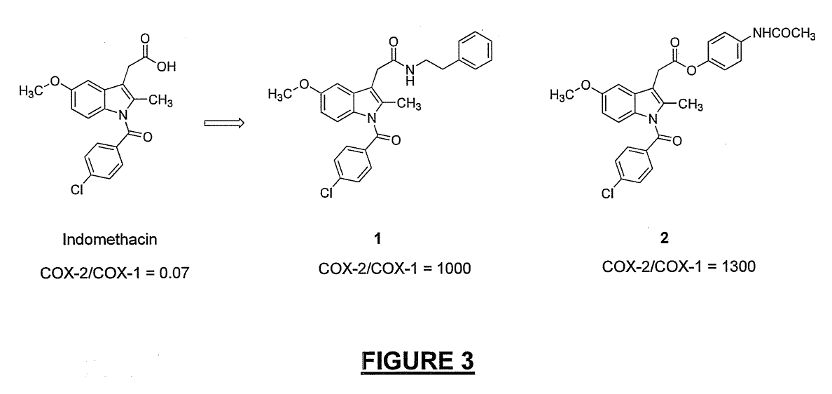

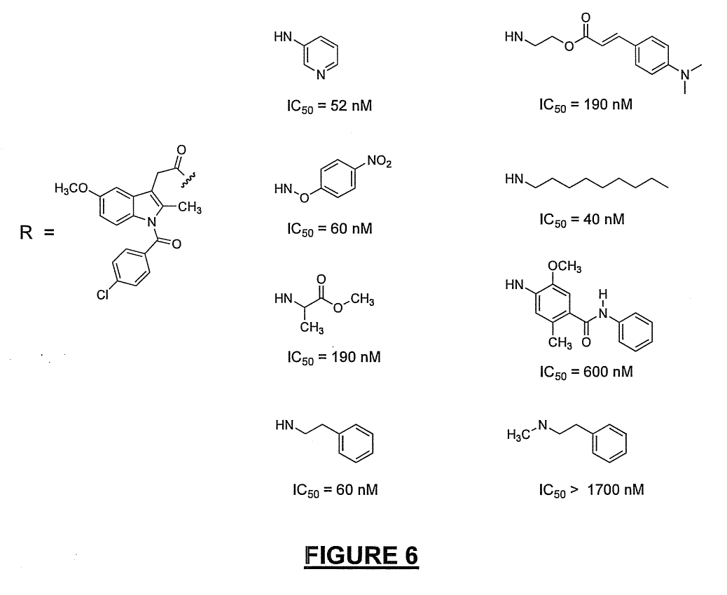

A major effort in the development of COX-2 inhibitors as derivatives of NSAIDs has focused on indomethacin as a parent compound. Indomethacin, which is approximately 15-fold more potent an inhibitor of COX-1 than COX-2, can be converted in a single step to amide or ester derivatives that exhibit COX-2 selectivities of greater than 1300-fold relative to COX-1 (FIG. 3; see also Kalgutkar et al., 2000b). Both amides and esters of indomethacin are active, and a large number of alkyl and aromatic substituents exhibit potent and selective COX-2 inhibition. FIG. 6 provides an example of some of the inhibitors that have been generated from the amidation of indomethacin, and illustrates the wide variety of structural moieties that are selective COX-2 inhibitors.

### II. COX-2-Selective Ligands

In some embodiments, the presently disclosed subject matter relates to a method for synthesizing a radiological imaging agent comprising combining a COX-2-selective ligand with a functional group comprising a detectable moiety, wherein the COX-2-selective ligand is a derivative of a non-steroidal anti-inflammatory drug (NSAID) comprising an ester moiety or a secondary amide moiety. Thus, the method provides for the synthesis of a bifunctional molecule: one function being the ability to selectively bind COX-2, and the other to be detectable by radiological or optical imaging.

As used herein, the phrase “COX-2-selective ligand” refers to a molecule that exhibits preferential binding to a COX-2 polypeptide. As used herein, “selective binding” means a preferential binding of one molecule for another in a mixture of molecules. The binding of an inhibitor to a target molecule can be considered selective if the binding affinity is about 1×10⁴ M⁻¹ to about 1×10⁶ M⁻¹ or greater. In some embodiments, a COX-2-selective ligand is a COX-2-selective inhibitor, a “COX-2-selective inhibitor” being defined as a molecule that inhibits the activity of COX-2 in relative excess of its inhibition of COX-1. In some embodiments, COX-2-selective ligands bind covalently to COX-2 polypeptides. In other embodiments, COX-2-selective ligands bind non-covalently to COX-2 polypeptides

In some embodiments, a COX-2-selective ligand is a derivative of a non-steroidal anti-inflammatory drug (NSAID). As used herein, the term “derivative” refers to a structural variant of a compound in which one or more atoms have been changed to yield a new compound containing one or more functional groups that differ from the parent compound. This change can occur by any suitable process, but typically occurs by reacting the NSAID with an intermediate, wherein a group is transferred from the intermediate to the NSAID to create a derivative.

NSAIDs that can be derivatized can intrinsically be COX-2 selective ligands. Alternatively, non-COX-2-selective NSAIDS can be converted into COX-2-selective ligands for use in the methods described herein. Methods for converting non-COX-2-selective NSAIDS into COX-2-selective ligands include the methods generally set forth in Kaigutkar et al., 1998a; and/or Kalgutkar et al., 1998b; and/or Kalgutkar et al., 2000a; and/or Kalgutkar et al., 2000b. These methods include, but are not limited to, methods for acetylating NSAIDs to make them COX-2-selective, and methods for converting NSAIDs into their respective neutral amide or ester derivatives to make them COX-2 selective. These methods are useful in making NSAID derivatives that covalently bind COX-2, as well as in making NSAID derivatives that non-covalently bind COX-2.

In some embodiments, the NSAID is selected from the group consisting of fenamic acids, indoles, phenylalkanoic acids, phenylacetic acids, pharmaceutically acceptable salts thereof, and combinations thereof. In some embodiments, the non-steroidal anti-inflammatory drug is selected from the group consisting of aspirin, o-(acetoxyphenyl)hept-2-ynyl sulfide (APHS), indomethacin, 6-methoxy-α-methyl-2-naphthylacetic acid, meclofenamic acid, 5,8,11,14-eicosatetraynoic acid (ETYA), diclofenac, flufenamic acid, niflumic acid, mefenamic acid, sulindac, tolmetin, suprofen, ketorolac, flurbiprofen, ibuprofen, aceloferac, alcofenac, amfenac, benoxaprofen, bromfenac, carprofen, clidanac, diflunisal, efenamic acid, etodolic acid, fenbufen, fenclofenac, fenclorac, fenoprofen, fleclozic acid, indoprofen, isofezolac, ketoprofen, loxoprofen, meclofenamate, naproxen, orpanoxin, pirprofen, pranoprofen, tolfenamic acid, zaltoprofen, zomepirac, and pharmaceutically acceptable salts thereof, and combinations thereof. In some embodiments, the non-steroidal anti-inflammatory drug is selected from the group consisting of aspirin, o-(acetoxyphenyl)hept-2-ynyl sulfide (APHS), indomethacin, meclofenamic acid, 5,8,11,14-eicosatetraynoic acid (ETYA), ketorolac, and pharmaceutically acceptable salts thereof, and combinations thereof.

In some embodiments, a COX-2 ligand is a derivative of an NSAID comprising an ester moiety or a secondary amide moiety. In some embodiments, a carboxylic acid group of the NSAID as been derivatized to an ester or a secondary amide. In some embodiments, the secondary amide derivative is selected from the group consisting of indomethacin-N-methyl amide, indomethacin-N-ethan-2-ol amide, indomethacin-N-octyl amide, indomethacin-N-nonyl amide, indomethacin-N-(2-methylbenzyl)amide, indomethacin-N-(4-methylbenzyl)amide, indomethacin-N—[(R)-α,4-dimethylbenzyl]amide, indomethacin-N-((S)-α,4-dimethylbenzyl)amide, indomethacin-N-(2-phenethyl)amide, indomethacin-N-(4-fluorophenyl)amide, indomethacin-N-(4-chlorophenyl)amide, indomethacin-N-(4-acetamidophenyl)amide, indomethacin-N-(4-methylmercapto)phenyl amide, indomethacin-N-(3-methylmercaptophenyl)amide, indomethacin-N-(4-methoxyphenyl)amide, indomethacin-N-(3-ethoxyphenyl)amide, indomethacin-N-(3,4,5-trimethoxyphenyl)amide, indomethacin-N-(3-pyridyl)amide, indomethacin-N-5-[(2-chloro)pyridyl]amide, indomethacin-N-5-[(1-ethyl)pyrazolo]amide, indomethacin-N-(3-chloropropyl)amide, indomethacin-N-methoxycarbonylmethyl amide, indomethacin-N-2-(2-L-methoxycarbonylethyl)amide, indomethacin-N-2-(2-D-methoxycarbonylethyl)amide, indomethacin-N-(4-methoxycarbonylbenzoyl)amide, indomethacin-N-(4-methoxycarbonylmethylphenyl)amide, indomethacin-N-(2-pyrazinyl)amide, indomethacin-N-2-(4-methylthiazolyl)amide, indomethacin-N-(4-biphenyl)amide, and combinations thereof.

Those skilled in the art will appreciate that an evaluation of the selectivity and efficacy of binding of the NSAID derivative to the COX-2 enzyme, e.g., after the derivative is synthesized, can be desirable. Methods of screening selective COX-2 inhibitors for activity can be carried out in vitro and/or in intact cells, and are known in the art. See e.g., Kalgutkar et al., 1998a; Kalgutkar et al., 1998b; Kalgutkar et al., 2000a; Kalgutkar et al., 2000b; Kalgutkar et al., 2002. One example of an in vitro screening method takes advantage of the fact that both human and murine recombinant COX-2 can be expressed and isolated in pure form from an Sf-9 cell expression system. Briefly, typical assays involve the incubation of COX-1 (44 nM) or COX-2 (66 nM) in a 200 μL reaction mixture containing 100 mM Tris-HCl, pH 8.0, 500 μM phenol and 50 μM ¹⁴C-arachidonic acid (55 mCi/mmol) for 30 seconds at 37° C. COX-1, which is not readily obtained in pure form from similar expression systems, can be purified from ovine seminal vesicles by standard procedures. Alternatively, membrane preparations from outdated human platelets can provide a source of human COX-1. The NSAID derivative(s) that is being screened for activity is added as a stock solution in dimethyl sulfoxide (DMSO) either concomitantly with the addition of arachidonic acid (to test for competitive inhibition) or for various periods of time prior to the addition of arachidonic acid (to test for time-dependent inhibition). The reaction is stopped by the addition of 200 μL of ethanol/methanol/1 M citrate, pH 4.0 (30:4:1). The extracted products are separated by thin layer chromatography (TLC), which allows quantitation of total product formation as well as assessment of product distribution. This assay is useful to define IC₅₀ values for inhibition of either enzyme, and to determine time-dependency of inhibition. It also provides information concerning changes in products formed as a result of inhibition.

While the TLC assay described above provides considerable information, it is labor-intensive for screening large numbers of candidate NSAID derivatives. Accordingly, as an alternative, a simplified assay can be used. Incubation conditions can be essentially as described above, except all candidate derivatives are first screened at a concentration of 1 mM with a preincubation time of 30 minutes. The substrate need not be radiolabeled, and the reaction can be stopped by the addition of 2 μL of formic acid. Product formation can be quantitated by enzyme-linked immunosorbent assay (ELISA) using commercially available kits. Compounds found to demonstrate potency and selectivity against COX-2 can optionally be further evaluated by the TLC assay. Other in vitro assay methods for screening NSAID derivatives for activity (e.g., selectivity for the COX-2 enzyme) can also be used by the skilled artisan.

As will be appreciated by the skilled artisan, activity in purified enzyme preparations as described above does not guarantee that an NSAID derivative will be effective in intact cells. Thus, NSAID derivatives that are identified as potentially useful in the methods described herein can be further tested using, for example, the RAW264.7 murine macrophage cell line. These cells are readily available (for example, from the American Type Culture Collection, Manassas, Va., United States of America) and are easily cultured in large numbers. They normally express low levels of COX-1 and very low to undetectable levels of COX-2. Upon exposure to bacterial lipopolysaccharide (LPS), however, COX-2 levels increase dramatically over the ensuing 24 hour period, and the cells produce PGD₂ and PGE₂ from endogenous arachidonic acid stores (generally, ˜1 nmol/10⁷ cells total PG formation). After LPS exposure, the addition of exogenous arachidonic acid results in the formation of additional PGD₂ and PGE₂ as a result of metabolism by the newly synthesized COX-2.

This system provides a number of approaches for testing the inhibitory potency of COX-2-selective ligands (e.g., inhibitors). In general, following LPS activation, cells can be treated for 30 minutes with the desired concentrations of candidate derivative(s) in DMSO. ¹⁴C-arachidonic acid can be added, and the cells can be incubated for 15 minutes at 37° C. Product formation can be assessed following extraction and TLC separation of the culture medium. Alternatively, the effects of candidate derivatives on PG synthesis from endogenous arachidonic acid can be assessed by incubating cells with desired concentrations of candidate derivatives 30 minutes prior to LPS exposure. Following a 24 hour incubation, medium can be collected and extracted, and the amount of PGD₂ and/or PGE₂ can be assayed by gas chromatography-mass spectrometry, liquid chromatography-mass spectrometry, or ELISA. The latter method can prove to be particularly useful, since NSAID derivatives are often found to be more potent when assayed for activity using endogenous arachidonic acid as opposed to exogenously supplied substrate.

The RAW264.7 assay is but one example of a cell-based assay for screening the activity of NSAID derivatives; the skilled artisan will appreciate that assays using alternative cell lines and methodologies can be used.

### III. Radiological and Optical Imaging Agents

Described herein are radiological and/or optical imaging agents that comprise COX-2-selective ligands and a detectable group. In certain embodiments, the COX-2-selective ligands are NSAID derivatives comprising an ester moiety or a secondary amide moiety. As used herein, the term “radiological imaging agent” refers to a compound that can be used to enhance the visualization of a tissue or cell using standard radiological or optical imaging techniques.

Methods of synthesizing inventive imaging agents are also described. In some embodiments, the present imaging agents are synthesized by reacting a COX-2-selective ligand with a compound comprising a detectable group. In certain embodiments, the COX-2-selective ligands are NSAID derivatives as described above. In still other certain embodiments, the NSAID derivatives comprise an ester moiety or a secondary amide moiety.

“Detectable groups”, as defined herein, are functional groups that can be detected by one or more spectroscopic techniques, as described herein. Representative spectroscopic techniques that can be used to detect radiological and/or optical imaging agents and detectable groups include, but are not limited to, those techniques that detect fluorescence; chemical and biological luminescence; visible, ultraviolet, X-ray, infrared, and microwave light wavelengths; radiation generated by radioisotopes (for example, ¹⁸F), and others. Specific techniques include, but are not limited to, scintigraphic imaging techniques (for example, positron emission tomography (PET), single photon emission computed tomography (SPECT), gamma camera imaging, and rectilinear scanning), near infrared luminescence (NIR), and monochromatic X-ray.

The skilled artisan will appreciate that the selection of a particular spectroscopic technique plays a role in determining the desired characteristics of the imaging agent and detectable groups, and the applicability of any particular embodiment described herein to the selected technique. Stated another way, the skilled artisan will understand that the choice of a detectable group in synthesizing an imaging agent can depend in whole or in part on the specific spectroscopic technique being employed.

Exemplary detectable groups include, but are not limited to, halogen-containing moieties, fluorescent moieties, metal ion-chelating moieties, dyes, radioisotope-containing moieties, and combinations thereof. In some embodiments, a halogen-containing moiety comprises a fluorine atom, an iodine atom, a bromine atom, or a radioactive isotope thereof.

For use in positron emission tomography, the detectable group comprises an appropriate positron-emitting source. The term “positron-emitting source” refers to an atom that emits a particle that can directly or indirectly be detected using a PET scanner. PET generally uses a short half-life, radioactively labeled substance introduced into the material to be scanned (for example, into a tumor present within a subject) for the purposes of the scan. This radioactive substance emits positrons, which, after annihilation with electrons, give rise to positron annihilation radiation, which can be detected using standard PET techniques. Representative positron-emitting sources include, but are not limited to, ¹¹C, ¹⁴O, ¹⁵O, ¹⁷F, ¹⁸F, ¹⁹Ne, ⁵²Fe, ⁶²Zn, ⁶⁴Cu, and ⁶⁸Ga, although other positron-emitting sources could also be employed.

For use in monochromatic X-ray detection, the detectable group will desirably comprise one or more iodine-containing moieties. Examples of such moieties include substituted benzene rings, in which at least one hydrogen has been replaced with iodine. In some embodiments, the iodine-containing moiety comprises a benzene ring with three hydrogens replaced by iodine.

For use in fluorescent detection, the detectable can be a fluorescent dye (e.g., a “fluorophore”). Many of these fluorescent dyes are commercially available, and include, but are not limited to, carbocyanine and aminostyryl dyes, long chain dialkyl carbocyanines (e.g., Dil, DiO, and DiD available from Molecular Probes Inc., Eugene, Oreg., United States of America), and dialkylaminostyryl dyes.

A fluorescent label can also comprise sulfonated cyanine dyes, including Cy5, Cy5.5, and Cy7 (available from Amersham Biosciences Corp., Piscataway, N.J., United States of America), IRD41 and IRD700 (available from Li-Cor, Inc., Lincoln, Nebr., United States of America), NIR-1 (available from Dejindo, Kumamoto, Japan), and LaJolla Blue (available from Diatron, Miami, Fla., United States of America). See also Licha et al., 2000; Weissleder et al., 1999; and Vinogradov et al., 1996.

In addition, a fluorescent label can comprise an organic chelate derived from lanthanide ions, for example fluorescent chelates of terbium and europium. See U.S. Pat. No. 5,928,627. Such labels can be conjugated or covalently linked to an NSAID derivative as disclosed therein. The chelator utilizes a number of coordinating atoms at coordination sites, as these terms are understood in the art, to bind the metal ion. The replacement of a coordination atom with a functional moiety to allow covalent attachment of the fluorescent label to a linker or other moiety might render the metal ion complex more toxic by decreasing the half-life of dissociation for the metal ion complex. Thus, in some embodiments, a site other than a coordination site is used for covalent attachment. However, for some applications, for example analysis of tumor tissue and the like, the toxicity of the metal ion complexes might not be of paramount importance and thus covalent attachment via a coordination site is appropriate.

Similarly, some metal ion complexes are so stable that even the replacement of one or more additional coordination atoms with a blocking moiety does not significantly affect the half-life of dissociation. For example, both diethylenetriamine pentaacetate (DTPA) and tetraazacyclododecyltetraacetic acid (DOTA), described hereinbelow, are extremely stable when complexed with Gd³⁺. Accordingly, one or several of the coordination atoms of the chelator can be replaced with one or more functional moieties for covalent attachment without a significant increase in toxicity.

There are a large number of known macrocyclic chelators or ligands that are used to chelate lanthanide and other metal ions. See e.g., Alexander, 1995; Jackels, 1990, expressly incorporated herein by reference, which describes a large number of macrocyclic chelators and their synthesis. Similarly, there are a number of patents that describe suitable chelators for use in the invention, including U.S. Pat. Nos. 5,155,215; 5,087,440; 5,219,553; 5,188,816; 4,885,363; 5,358,704; 5,262,532; and Meyer et al., 1990, all of which are also expressly incorporated by reference. There are a variety of factors that influence the choice and stability of the chelate metal ion complex, including enthalpy and entropy effects (for example number, charge and basicity of coordinating groups, ligand field and conformational effects, etc.). In general, the chelator has a number of coordination atoms that are capable of binding the metal ion. The number of coordination atoms, and thus the structure of the chelator, depends on the metal ion. Thus, as will be understood by those in the art, any of the known metal ion chelators or lanthanide chelators can be easily modified using the teachings herein to add a functional moiety for covalent attachment to an optical dye or linker.

For in vivo detection of a fluorescent label, an image is created using emission and absorbance spectra that are appropriate for the particular label used. The image can be visualized, for example, by diffuse optical spectroscopy. Additional methods and imaging systems are described in U.S. Pat. Nos. 5,865,754; 6,083,486; and 6,246,901, among other places.

Near infrared (NIR) light that can penetrate tissue several centimeters, and fluorescent contrast agents responsive to NIR light can be used to provide a viable imaging system. For use in luminescent detection, the detectable group can be a chemical dye. Dyes that can be used include, but are not limited to, the class of polymethine dyes selected from the following group: cyanine, styryl, merocyanine, squaraine, and oxonol dyes. Representative dyes of the class of cyanine dyes having maximum absorption and fluorescence values between 700 and 1000 nm and extinction coefficients of about 140,000 l M⁻¹ cm⁻¹ and more, and carrying one or several unsubstituted, branched or non-branched, acyclic or cyclic or, optionally, aromatic carbon-hydrogen residues and/or containing oxygen, sulfur, nitrogen. For example, a dye can contain a cyanine, styryl, merocyanine, squaraine, or oxonol dye, or a mixture of said dyes. For example, cyanine dyes with intense absorption and emission in the near-infrared (NIR) region are particularly useful because biological tissues are optically transparent in this region (Wilson, 1991). For example, indocyanine green, which absorbs and emits in the NIR region, has been used for monitoring cardiac output, hepatic functions, and liver blood flow (He et al., 1998; Caesar et al., 1961), and its functionalized derivatives have been used to conjugate biomolecules for diagnostic purposes (Mujumdar et al., 1993). See also U.S. Pat. Nos. 5,453,505 and 6,403,625; WO 98/48846; WO 98/22146; WO 96/17628; WO 98/48838.

In some embodiments, a radiological imaging agent of the presently disclosed subject matter comprises the following structure:

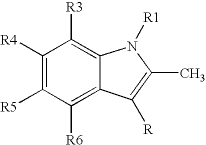

wherein
R is selected from the group consisting of

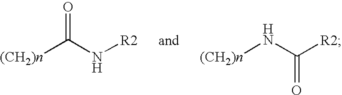

R1 is selected from the group consisting of a detectable group,

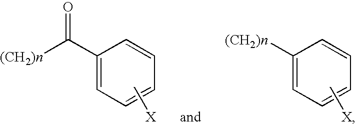

wherein X is a halogen or a radioactive isotope thereof at one or more positions of the aromatic ring;
R2 comprises a detectable group or a halo substituted aryl;
R3, R4, R5, and R6 are each independently selected from the group consisting of hydrogen; halo; C₁ to C₆ alkyl or branched alkyl;
C₁ to C₆ alkoxy or branched alkoxy; benzyloxy; SCH₃; SOCH₃; SO₂CH₃; SO₂NH₂; and CONH₂;
n is 0-5 inclusive;
   and wherein at least one of R1 and R2 comprises a detectable group. Thus, n can be 0, 1, 2, 3, 4, or 5.

In some embodiments, a radiological imaging agent of the presently disclosed subject matter comprises the following structure

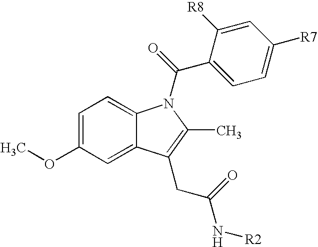

wherein R7 comprises a halogen and R8 is selected from the group consisting of hydrogen, a halogen, C₁-C₆ alkyl or branched alkyl, and C₁-C₆ aryl or branched aryl.

As used herein, the term “halogen” refers to one of the atoms of column VII of the Periodic Table of the Elements, and thus includes fluorine (F), chlorine (Cl), bromine (Br), iodine (I), and astatine (At). In some embodiments, a halogen is F, in some embodiments, a halogen is Cl, and in some embodiments a halogen is Br. As used herein, the term “halogen” refers to all isotopes of F, Cl, Br, I, and At including, but not limited to radioactive isotopes. In some embodiments, a halogen is ¹⁸F.

In some embodiments, R2 has the following structure:

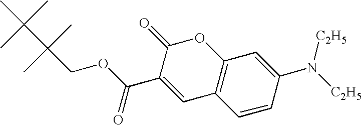

In some embodiments, R2 has the following structure:

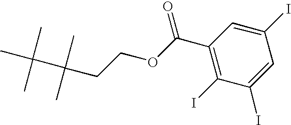

In some embodiments, R2 has the following structure:

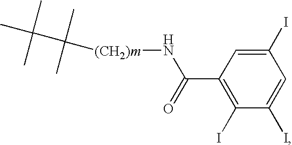

wherein m=an integer between 0 and 8, inclusive. Thus, m can be 0, 1, 2, 3, 4, 5, 6, 7, or 8.

In some embodiments, R2 has the following structure:

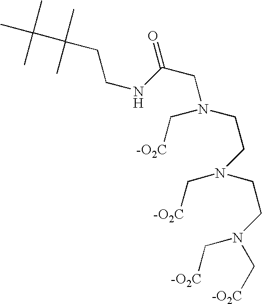

In some embodiments of this structure, the imaging agent further comprises a coordinated metal ion. In some embodiments, the coordinated metal ion is selected from the group consisting of Gd³⁺, Fe³⁺, Mn²⁺, Yt³⁺, Dy³⁺, and Cr³⁺. In some embodiments, the coordinated metal ion is Gd³.

In some embodiments of the instant radiological imaging agent, R1 is Cl and R2 has the following structure:

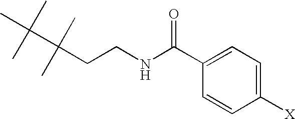

wherein X is a halogen or a radioactive isotope thereof. In some embodiments, X is ¹⁸F.

In some embodiments of the present imaging agent, R2 has the following structure:

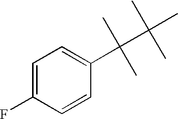

In some embodiments, R2 has the following structure:

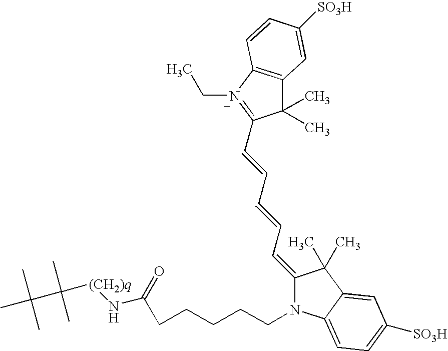

wherein q=an integer between 0 and 8, inclusive. Thus, q can be 0, 1, 2, 3, 4, 5, 6, 7, or 8.

In some embodiments, the radiological imaging agent comprises the following structure:

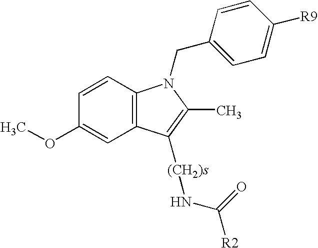

wherein R9 is a halogen, R2 is p-halobenzene, and s=1-4. Thus, s can be 0, 1, 2, 3, or 4. In some embodiments, R1 is Br, s=2, and R2 is p-¹⁸F-benzene.

In some embodiments, a radiological imaging agent of the presently disclosed subject matter comprises the following structure:

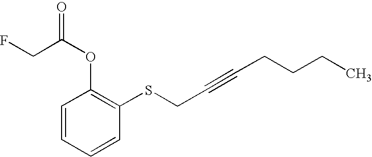

In some embodiments of the current radiological imaging agent, the fluorine atom is ¹⁸F.

In some embodiments, a radiological imaging agent of the presently disclosed subject matter comprises the following structure:

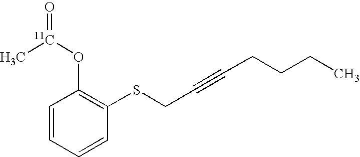

In some embodiments, a radiological imaging agent of the presently disclosed subject matter comprises the following structure:

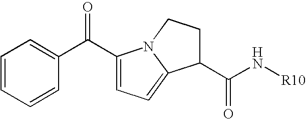

wherein R10 comprises a detectable group. In some embodiments, R10 has the following structure:

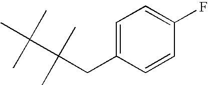

In some embodiments, a radiological imaging agent of the presently disclosed subject matter comprises the following structure:

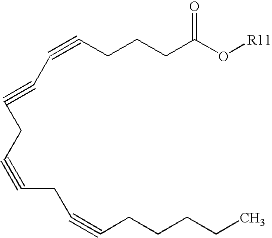

wherein R11 comprises a detectable group selected from the group consisting of a halogen-containing moiety, a fluorescent moiety, a metal ion-chelating moiety, a dye, a radioisotope-containing moiety, and combinations thereof.

In some embodiments, a radiological imaging agent of the presently disclosed subject matter comprises the following structure:

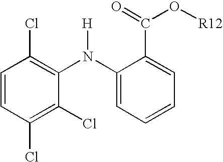

wherein R12 comprises a detectable group selected from the group consisting of a halogen-containing moiety, a fluorescent moiety, a metal ion-chelating moiety, a dye, a radioisotope-containing moiety, and combinations thereof.

In some embodiments, the radiological imaging agent comprises a detectable group and an indomethacin derivative selected from the group consisting of Compounds 355, 360, and 389, wherein Compounds 355, 360, and 389 have the following structures:

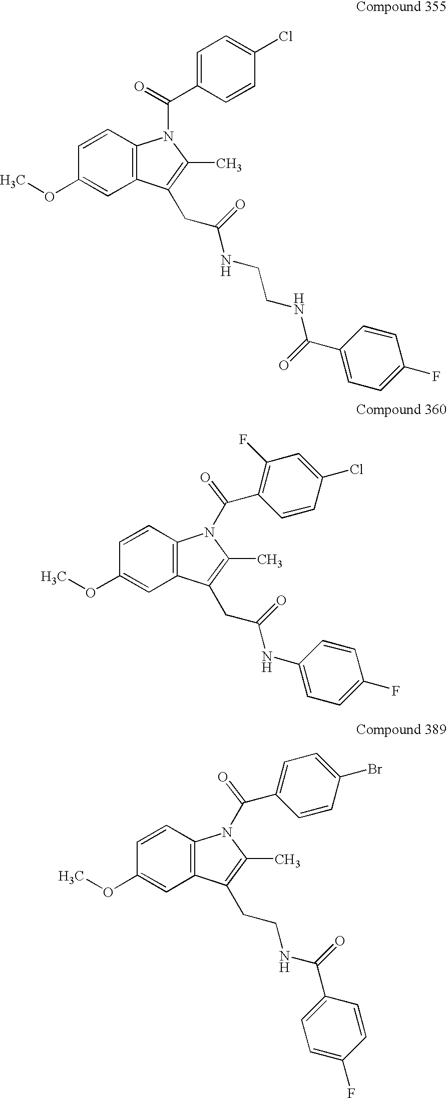

In some embodiments of the instant radiological imaging agent, the detectable group is ¹⁸F, and one or more fluorine atoms present in Compounds 355, 360, or 389 is ¹⁸F.

Radiological imaging compounds described herein can optionally be evaluated by the skilled artisan for efficacy and suitability for a selected detection method. Such methods are known in the art and/or can be easily ascertained by the skilled artisan. For example, a synthesized radiological imaging compound can be evaluated as an imaging agent in intact cells. For such evaluations, mouse resident peritoneal macrophages (MPM) can be used. These cells normally possess relatively high quantities of COX-1, and low to undetectable quantities of COX-2 after isolation and overnight culture. However, following exposure to LPS, MPM show a rapid synthesis of COX-2 that begins within 1 hour and reaches a peak at 6 to 8 hours. Concomitantly, these cells produce large quantities of prostacyclin (identified as its decomposition product, 6-ketoPGF1a) and PGE₂. Thus, MPM respond to LPS more rapidly than do RAW264.7 cells, and produce larger quantities and different classes of PG products.

Quantitative western blot analysis of cell lysates have shown that after 6 hours of LPS treatment, MPM cells might contain as many as 10⁵-10⁶ molecules of COX-2 per cell, indicating a high concentration of the imaging target compound. Because COX-1 levels remain constant during this time, LPS-treated MPM contain both isoforms of the enzyme, whereas untreated MPM contain only COX-1. Thus, a comparison of the effects of imaging agents in LPS-treated versus untreated cells allows one to control for any effects due to binding to COX-1. Furthermore, mice bearing a targeted gene deletion of either the COX-1 or the COX-2 gene are available (S. K. Dey, Vanderbilt University, Nashville, Tenn., United States of America; see Langenbach et al., 1995; Morham et al., 1995). MPM from these mice can serve as valuable controls to verify that effects of imaging agents are due specifically to COX-2.

MPM can be isolated from wild-type mice, or those bearing a targeted gene deletion by peritoneal lavage using well-established techniques. The cells are readily purified by adherence and cultured overnight. Following incubation for 6 hours in the presence or absence of LPS, cells can be treated for the desired period with inhibitors, then the appropriate imaging modality can be used to test the effectiveness of the test agent.

MPM-based screening assays can be tailored and optimized by the skilled artisan based on the kind of imaging agent being evaluated and the kind of detection technique being used. For example, radiological imaging agents comprising multiple iodine atoms for monochromatic X-ray can be tested. For the testing of these compounds, cells that have or have not been exposed to LPS can be treated with test compound, and then removed from the culture dishes and centrifuged, creating a cell button at the base of the centrifuge tube. Similar cultures of cells, which have not been exposed to the iodinated agent, can be treated identically. The tubes can then be suspended in a water phantom and 3-dimensionally imaged using the monochromatic X-ray beam tuned to the iodine k-edge (33.3 kiloelectron volts (keV)). Attenuation characteristics of the computed tomography (CT) images of the cell buttons can be established to determine whether or not the intracellular iodine has created a detectable signal to differentiate cells exposed to inhibitor from those not exposed, and to differentiate LPS-treated from untreated cells.

Radiological imaging compounds synthesized for optical imaging techniques can similarly be evaluated. Briefly, cells are examined after treatment with candidate fluorescent or chelating agents. These cells can be examined in suspension (by spectroscopy) or after adhering to coverslips (microscopy). Quantitative measurements of fluorescence signals can be performed in the presence and absence of background (i.e. by adding untreated cells).

For PET imaging agents radiolabeled with ¹⁸F, cells can be washed and scraped from culture dishes following incubation with inhibitors and the amount of radioactivity taken up can be determined by counting in an automated well scintillation γ-counter. Other screening methods for these agents can also be employed.

The in vivo efficacy of radiological imaging agents described herein can also be evaluated. For example, imaging agents can be evaluated for their ability to image COX-2-expressing tumors in vivo. Assays for this kind of evaluation are known in the art, and include, but are not limited to, the use of the HCA-7 human colon carcinoma xenograft model (see e.g., Sheng et al., 1997; Williams et al., 2000b; Mann et al., 2001); the murine Lewis lung carcinoma model (see e.g, Stolina et al., 2000; Eli et al., 2001); and murine colorectal carcinoma models that include, but are not limited to, the APC^{Min-} mouse model (see Su et al., 1992; Moser et al., 1995; Boolbol et al., 1996; Williams et al., 1996; Barnes and Lee, 1998; Jacoby et al., 2000; Oshima et al., 1996) and the azoxymethane-induced colon carcinoma model (Fukutake et al. 1998).

The term “independently selected” is used herein to indicate that the R groups, e.g., R¹, R², R³, etc. can be identical or different (e.g., R¹, R² and R³ can all be substituted alkyls, or R¹ and R⁴ can be a substituted alkyl and R³ can be an aryl, etc.). Moreover, “independently selected” means that in a multiplicity of R groups with the same name, each group can be identical to or different from each other (e.g., one R¹ can be an alkyl, while another R¹ group in the same compound can be aryl; one R² group can be H, while another R² group in the same compound can be alkyl, etc.).

A named R group will generally have the structure that is recognized in the art as corresponding to R groups having that name. For the purposes of illustration, representative R groups as enumerated above are defined herein. These definitions are intended to supplement and illustrate, not preclude, the definitions known to those of skill in the art.

As used herein, the term “alkyl” means C₁₋₁₀ inclusive (i.e. carbon chains comprising 1, 2, 3, 4, 5, 6, 7, 8, 9, or 10 carbon atoms; also, in some embodiments, C₁₋₆ inclusive, i.e. carbon chains comprising 1, 2, 3, 4, 5, or 6 carbon atoms) linear, branched, or cyclic, saturated or unsaturated (i.e., alkenyl and alkynyl)hydrocarbon chains, including for example, methyl, ethyl, propyl, isopropyl, butyl, isobutyl, tert-butyl, pentyl, hexyl, ethenyl, propenyl, butenyl, pentenyl, hexenyl, butadienyl, propynyl, butynyl, pentynyl, hexynyl, and allenyl groups.

The alkyl group can be optionally substituted with one or more alkyl group substituents which can be the same or different, where “alkyl group substituent” includes alkyl, halo, arylamino, acyl, hydroxy, aryloxy, alkoxyl, alkylthio, arylthio, aralkyloxy, aralkylthio, carboxy, alkoxycarbonyl, oxo and cycloalkyl. In this case, the alkyl can be referred to as a “substituted alkyl”. Representative substituted alkyls include, for example, benzyl, trifluoromethyl, and the like. There can be optionally inserted along the alkyl chain one or more oxygen, sulfur or substituted or unsubstituted nitrogen atoms, wherein the nitrogen substituent is hydrogen, alkyl (also referred to herein as “alkylaminoalkyl”), or aryl. Thus, the term “alkyl” can also include esters and amides. “Branched” refers to an alkyl group in which an alkyl group, such as methyl, ethyl, or propyl, is attached to a linear alkyl chain.

The term “aryl” is used herein to refer to an aromatic substituent, which can be a single aromatic ring or multiple aromatic rings that are fused together, linked covalently, or linked to a common group such as a methylene or ethylene moiety. The common linking group can also be a carbonyl as in benzophenone or oxygen as in diphenylether or nitrogen in diphenylamine. The aromatic ring(s) can include phenyl, naphthyl, biphenyl, diphenylether, diphenylamine, and benzophenone among others. In particular embodiments, the term “aryl” means a cyclic aromatic comprising about 5 to about 10 carbon atoms, including 5 and 6-membered hydrocarbon and heterocyclic aromatic rings.

An aryl group can be optionally substituted with one or more aryl group substituents which can be the same or different, where “aryl group substituent” includes alkyl, aryl, aralkyl, hydroxy, alkoxyl, aryloxy, aralkoxyl, carboxy, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene and —NR′R″, where R′ and R″ can be each independently hydrogen, alkyl, aryl and aralkyl. In this case, the aryl can be referred to as a “substituted aryl”. Also, the term “aryl” can also included esters and amides related to the underlying aryl group.

Specific examples of aryl groups include but are not limited to cyclopentadienyl, phenyl, furan, thiophene, pyrrole, pyran, pyridine, imidazole, isothiazole, isoxazole, pyrazole, pyrazine, pyrimidine, and the like.

The term “alkoxy” is used herein to refer to the —OZ¹ radical, where Z¹ is selected from the group consisting of alkyl, substituted alkyl, cycloalkyl, substituted cycloalkyl, heterocycloalkyl, substituted heterocycloalkyl, silyl groups and combinations thereof as described herein. Suitable alkoxy radicals include, for example, methoxy, ethoxy, benzyloxy, t-butoxy, etc. A related term is “aryloxy” where Z¹ is selected from the group consisting of aryl, substituted aryl, heteroaryl, substituted heteroaryl, and combinations thereof. Examples of suitable aryloxy radicals include phenoxy, substituted phenoxy, 2-pyridinoxy, 8-quinalinoxy, and the like.

The term “amino” is used herein to refer to the group —NZ¹Z², where each of Z¹ and Z² is independently selected from the group consisting of hydrogen; alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof. Additionally, the amino group can be represented as N⁺Z¹ Z² Z³, with the previous definitions applying and Z³ being either H or alkyl.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Aroyl” means an aryl-CO— group wherein aryl is as previously described. Exemplary aroyl groups include benzoyl and 1- and 2-naphthoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, lower alkyl, or aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Aralkyl” refers to an aryl-alkyl-group wherein aryl and alkyl are as previously described. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group as previously described. Exemplary alkylamino groups include ethylmethylamino, dimethylamino, and diethylamino.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl as previously described.

“Dialkylcarbamoyl” refers to a R′RN—CO— group wherein each of R and R′ is independently alkyl as previously described.

“Acyloxyl” refers to an acyl-O— group wherein acyl is as previously described.

“Acylamino” refers to an acyl-NH— group wherein acyl is as previously described.

“Aroylamino” refers to an aroyl-NH— group wherein aroyl is as previously described.

The term “amino” refers to the —NH₂ group.

The term “carbonyl” refers to the —(C═O)— group.

The term “carboxyl” refers to the —COOH group.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

### IV. Indomethacin-Based PET Contrast Agents

IV.A. General Considerations

The elevated expression of COX-2 in benign and malignant tumors and the apparent functional role that the enzyme plays in tumor growth suggests that COX-2 is an attractive target for the development of tumor-selective agents. The development of COX-2 selective indomethacin analogs has been accomplished by converting in one-step the non-selective COX-1 and COX-2 inhibitor indomethacin, into highly selective COX-2 inhibitors (see Kalgutkar et al., 2000b). The enhanced selectivity results from the conversion of the carboxylic acid functionality into amides and esters. In some cases, derivatives exhibit COX-2 selectivity greater than 1000-fold over COX-1. Therefore, in some embodiments of the presently disclosed subject matter, the development of COX-2 selective imaging agents centered primarily on a 5-methoxy-2-methylindole core, the main constituent of indomethacin. Additional strategies for synthesizing indomethacin derivatives for use as starting materials for the production of indomethacin-based PET contrast agents are disclosed in U.S. Pat. Nos. 6,207,700; 6,306,890; and 6,399,647.

In some embodiments, provided is the development of an indomethacin derivative PET agent. Positron emission tomography offers the highest spatial and temporal resolution of all nuclear medicine imaging modalities and allows quantitation of tracer concentrations in tissues. Of all the radioactive isotopes for PET, ¹⁸F is the most practical to work with due to its relatively low positron emission energy (maximum 635 KeV) and shortest positron linear range in tissue (2.3 mm) resulting in the highest resolution in PET imaging. Furthermore, its half-life (109.8 min) is long compared to other radioisotopes for relatively complex synthetic protocols and extended imaging sessions.

Despite the advantages of the modality, ¹⁸F radionuclide synthesis is challenging due to ¹⁸F's inherent half-life and radiation hazards. As such, all methods and manipulations of ¹⁸F should be simple and ideally automatable. Optimally, the incorporation of the radioisotope should be at the end of the synthesis. For this reason, nucleophilic aromatic substitution is the method of choice for the incorporation of the ¹⁸F anion into PET radioligand precursors. The exchange reaction is only possible, however, if activated (electron deficient) aromatics are used. Representative examples of suitable electron withdrawing groups on the aromatic moiety include the nitro, cyano, and carboxyl groups. Equally important is the presence of a suitable leaving group, with the trimethylammonium triflate salt being particularly useful.

Due to the short half-life of ¹⁸F (2 hours), PET agents must be prepared such that the ¹⁸F is incorporated at or near the end of the synthesis. Therefore, an ¹⁸F precursor that is one step away from the final product is desirable. The precursors that have been designed incorporate known leaving groups that have proven to exchange with ¹⁸F⁻ under the appropriate nucleophilic conditions of this reaction. The trimethylammonium triflate and tosylate are efficient precursors, with the nitro and halo groups also being useful.

IV.B. Indolyl Amide Series Indomethacin Derivatives

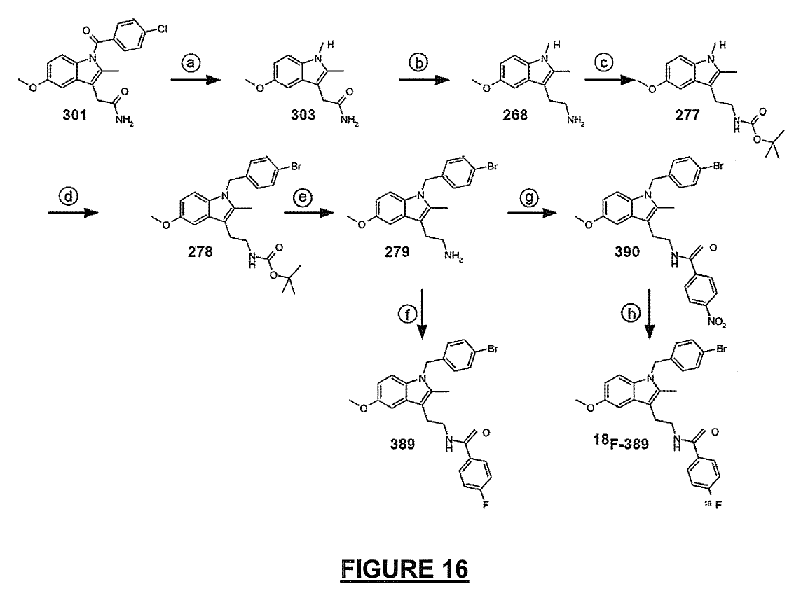

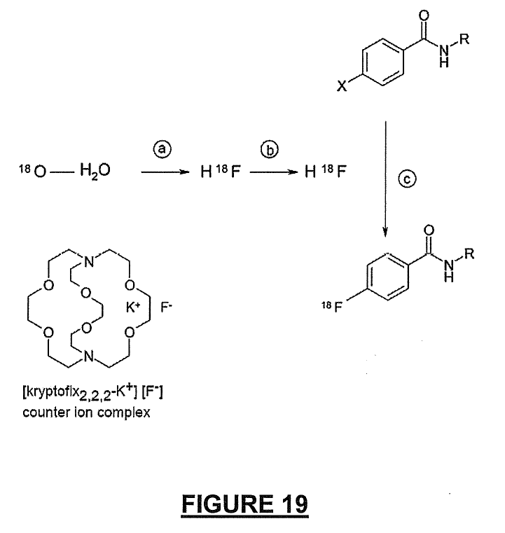

A generalized scheme for producing indomethacin derivatives in the indolyl amide series is shown in FIG. 16. As shown in FIG. 16 and described in Example 7, indomethacin can be converted through a series of steps to N-{2-[1-(4-Bromo-benzyl)-5-methoxy-2-methyl-1H-indol-3-yl]-ethyl}-4-nitro-benzamide (Compound 389). Compound 389 can then be labeled with ¹⁸F using the strategy shown in FIG. 19 to create a PET contrast agent that is specific for COX-2 (¹⁸F-labeled Compound 389).

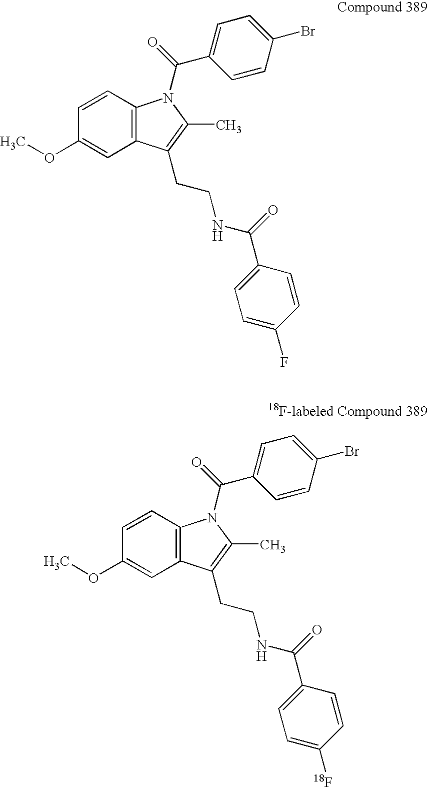

IV.C. Diamide Series Indomethacin Derivatives

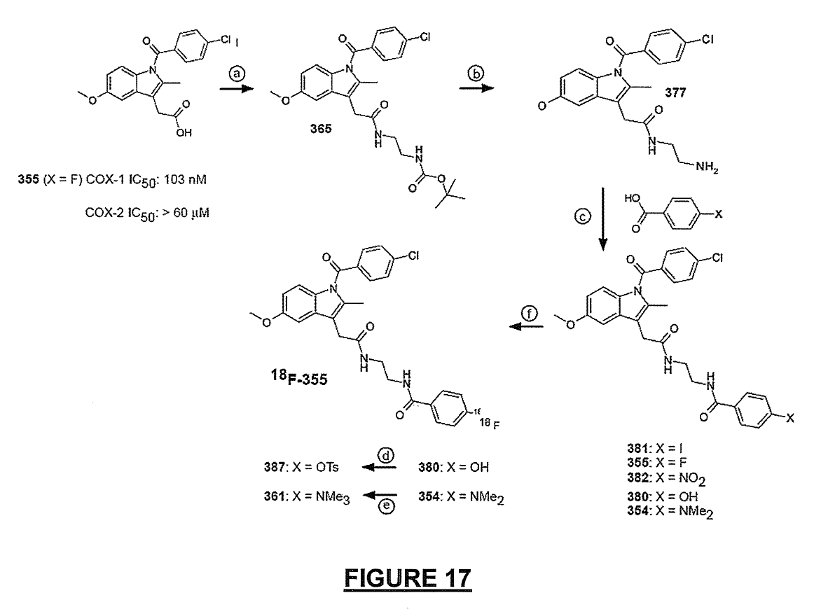

A generalized scheme for producing indomethacin derivatives in the diamide series is shown in FIG. 17. As shown in FIG. 17 and described in Example 8, indomethacin can be converted through a series of steps to N-(2-{2-[1-(4-Chloro-benzoyl)-5-methoxy-2-methyl-1H-indol-3-yl]-acetylamino}-ethyl)-4-fluoro-benzamide (Compound 355). Compound 355 can then be labeled with ¹⁸F using the strategy shown in FIG. 19 to create a PET contrast agent that is specific for COX-2 (¹⁸F-labeled Compound 355).

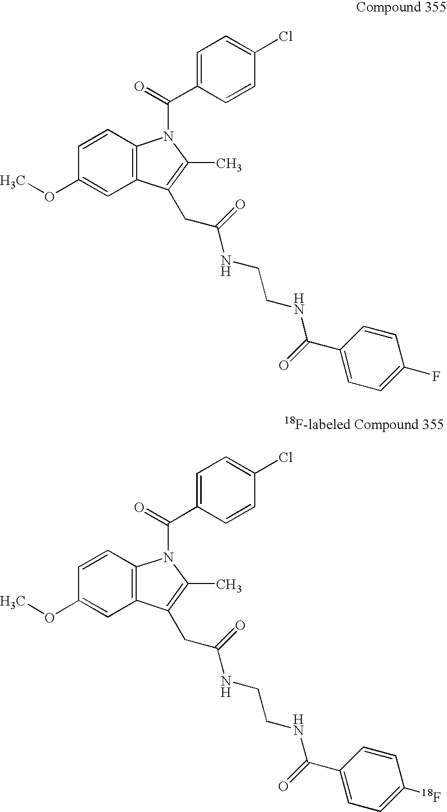

IV.D. Amide Series Indomethacin Derivatives

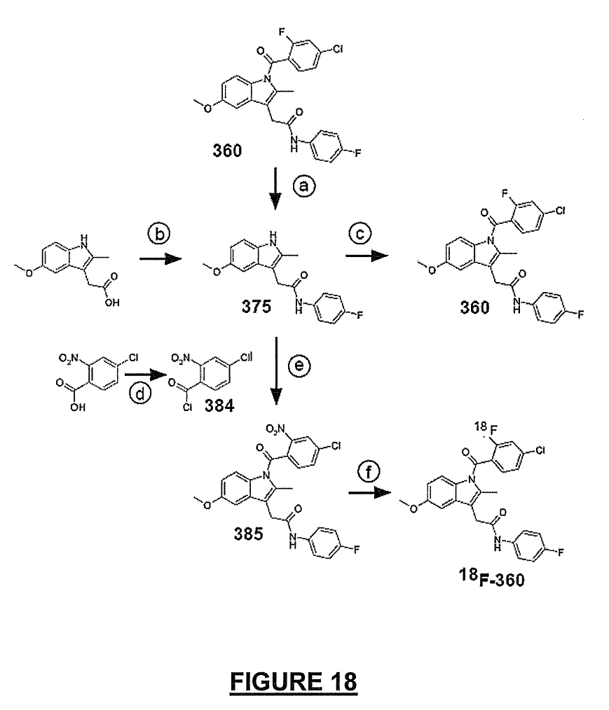

A generalized scheme for producing indomethacin derivatives in the amide series is shown in FIG. 18. As shown in FIG. 18 and described in Example 9, 5-methoxy-2-methyl-1H-indoleacetic acid or Compound 360, an indomethacin derivative synthesized by the co-inventors, can be converted through a series of steps to 2-[1-(4-Chloro-2-nitro-benzoyl)-5-methoxy-2-methyl-1H-indol-3-yl]-N-(4-fluoro-phenyl)-acetamide (Compound 385). Compound 385 can then be labeled with ¹⁸F using the strategy shown in FIG. 19 to create a PET contrast agent that is specific for COX-2 (¹⁸F-labeled Compound 360).

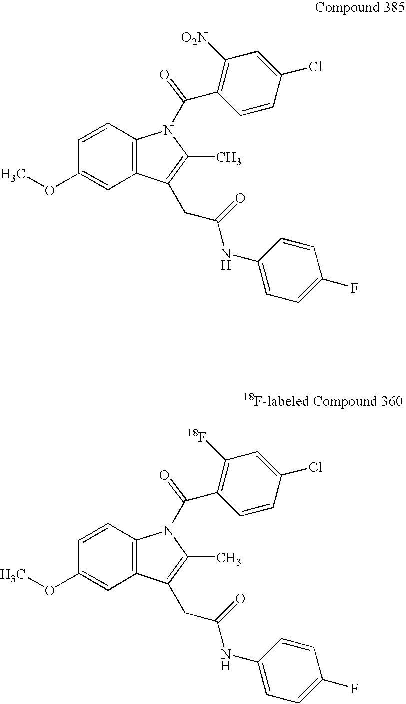

### V. Methods of Use

The presently disclosed subject matter also includes methods for imaging a target tissue in a subject, the method comprising (a) administering to the subject a radiological imaging agent under conditions sufficient for binding of the radiological imaging agent to the target tissue, wherein the radiological imaging agent comprises a COX-2-selective derivative of a non-steroidal anti-inflammatory drug (NSAID) comprising an ester moiety or a secondary amide moiety and further comprises a detectable group; and (b) detecting the detectable group in the target tissue.

The term “target tissue” refers to any cell or group of cells present in a subject. This term includes single cells and populations of cells. The term includes, but is not limited to, cell populations comprising glands and organs such as skin, liver, heart, kidney, brain, pancreas, lung, stomach, and reproductive organs. It also includes, but is not limited to, mixed cell populations such as bone marrow. Further, it includes but is not limited to such abnormal cells as neoplastic or tumor cells, whether individually or as a part of solid or metastatic tumors. The term “target tissue” as used herein additionally refers to an intended site for accumulation of a ligand following administration to a subject. For example, the methods of the present invention employ a target tissue comprising a tumor. In some embodiments, the target tissue is selected from the group consisting of an inflammatory lesion, a tumor, a neoplastic cell, a pre-neoplastic cell, and a cancer cell. In some embodiments, the inflammatory lesion is selected from the group consisting of a colon polyp and Barrett's esophagus.

As used herein, the term “cancer” encompasses cancers in all forms, including polyps, neoplastic cells, and pre-neoplastic cells.

As used herein, the term “neoplastic” is intended to refer to its ordinary meaning, namely aberrant growth characterized by abnormally rapid cellular proliferation. In general, the term “neoplastic” encompasses growth that can be either benign or malignant, or a combination of the two.

The term “tumor” as used herein encompasses both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas. The term “tumor” also encompasses radioresistant tumors, including radioresistant variants of any of the tumors listed above.

In some embodiments, the tumor is selected from the group consisting of a primary tumor, a metastasized tumor, and a carcinoma.

The methods and compositions of the presently claimed subject matter are useful for radiological imaging of a target tissue in any subject. Thus, the term “subject” as used herein includes any vertebrate species, for example, warm-blooded vertebrates such as mammals and birds. More particularly, the methods of the present invention are contemplated for the treatment of tumors in mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants and livestock (such as cattle, oxen, sheep, giraffes, deer, goats, bison, and camels), and horses. Also contemplated is the treatment of birds, including those kinds of birds that are endangered or kept in zoos, as well as fowl, and more particularly domesticated fowl or poultry, such as turkeys, chickens, ducks, geese, guinea fowl, and the like, as they are also of economic importance to humans. In some embodiments, the subject is a mammal. In some embodiments, the mammal is a human.

In some embodiments, the administering is peroral. In some embodiments, the administering is intravenous. In some embodiments, the administering is intraperitoneal. In some embodiments, the administration is intramuscular. In some embodiments, the administration is rectal. In some embodiments, the administration is by inhalation. In some embodiments, the administering is intratumoral. In some embodiments, a COX-2-selective ligand comprising a detectable group is administered intratumorally, and the tumor is visualized using PET.

### EXAMPLES

The following Examples provide illustrative embodiments. Certain aspects of the following Examples are described in terms of techniques and procedures found or contemplated by the present inventors to work well in the practice of the embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Synthesis of Aspirin-Derived COX-2-Selective Ligands

Aspirin is a representative NSAID that has significant analgesic properties. It is the only NSAID that covalently modifies cyclooxygenases. Aspirin acetylates a serine residue (Ser530 of COX-1 and Ser516 of COX-2), which appears to block the active site of the enzyme for its substrates (Van der Ouderaa et al., 1980; DeWitt et al., 1990), thereby inactivating the enzyme. While aspirin acetylates both COX-1 and COX-2, it is about 10-100 times as potent against COX-1 as it is against COX-2 (Meade et al., 1993; Vane and Botting, 1996).

Various derivatives of aspirin were investigated for their abilities to inhibit COX-1 and COX-2 in an effort to identify derivatives that displayed enhanced COX-2 inhibition relative to COX-1 inhibition. A series of acetoxybenzenes were derivatized in the ortho position with alkylsulfides. o-(Acetoxyphenyl)methyl sulfide exhibited moderate inhibitory potency and selectivity for COX-2 (Kalgutkar et al., 1998a). Variations in the acyl group, alkyl group, aryl substitution pattern, and heteroatom identity were also performed.

The compound that offered the best combination of potency and COX-2 selectivity was o-(acetoxyphenyl)hept-2-ynyl sulfide (APHS). IC₅₀ values for the inhibition of COX-2 and COX-1 by APHS are 0.8 μM and 17 μM, respectively. Like aspirin, APHS acetylates COX-2 at Ser516, and the time course for acetylation corresponds closely to the time course for irreversible inactivation of enzyme activity. Complete inactivation is achieved within about 30 min (k_{inact}/Kᵢ˜0.18 min⁻¹ μM⁻¹). Consistent with the proposed mechanism of action, the S516A mutant of COX-2 is resistant to the inhibitory effects of APHS (Kalgutkar et al., 1998a).

APHS is an effective inhibitor of COX-2 activity in the RAW 264.7 murine macrophage cell line activated by lipopolysaccharide (LPS) treatment. The IC₅₀ for inhibition of PGD₂ synthesis in response to addition of exogenous arachidonic acid is 0.12 μM. Furthermore, APHS inhibits the growth in soft agar of HCA-7 colon cancer cells (IC₅₀=2 μM), which express high levels of COX-2, and are dependent on COX-2 activity for maximal growth. In contrast, APHS has no effect on the growth of HCT-15 colon cancer cells, which do not express COX-2 (Kalgutkar et al., 1998a).

Two in vivo models of inflammation have been used to assess the effectiveness of COX-2 selective inhibitors. The first is the rat carageenan footpad model. Maximal edema is obtained in this model 3 hours after carageenan injection. APHS inhibits edema formation with an ED₅₀ of 6 mg/kg (p.o.). The ED₅₀ for inhibition by aspirin is 125 mg/kg. APHS induces no gastric toxicity at doses of 100 mg/kg whereas 50% of the animals treated with 100 mg/kg aspirin develop gastric lesions.

The second model used to evaluate in vivo efficacy is the rat air pouch model. In this model, a subcutaneous air pouch is infused with carageenan to establish a local inflammatory response. PGE₂ produced in the exudate is primarily the result of COX-2 activity, whereas thromboxane A₂ (TXA2) produced by blood platelets is the result of COX-1 activity. Thus, the selectivity of an inhibitor can be directly evaluated. In this model, APHS reduces PGE₂ levels in the pouch exudate by 95% at a dose of 5 mg/kg. This dose has no effect on serum thromboxane B₂ (TXB₂) levels. At a dose of 50 mg/kg, APHS reduces pouch PGE₂ and serum TXB₂ levels by 100% and 11%, respectively. These results contrast with those obtained with a 2 mg/kg dose of indomethacin, which reduces PGE₂ and TXB₂ levels by 100%, and 90%, respectively. Thus, APHS is a potent and selective COX-2 inhibitor in vivo (Kalgutkar et al., 1998a). It is noteworthy that daily oral administration of APHS to Sprague-Dawley rats at a dose of 100 mg/kg induces no detectable toxicities at 14 days as judged by gross or histopathological evaluation.

### Example 2

### Fluoro Analogs of APHS

The ability of APHS to selectively acetylate COX-2 provides multiple opportunities for the design of a PET imaging agent. From a technical standpoint, the most easily accomplished is to synthesize an isotopically labeled haloalkyl derivative of APHS. This requires that such derivatives must be effective inhibitors of COX-2. To explore this possibility, a fluoroacetyl derivative of APHS (F-APHS) was synthesized and shown to be an effective inhibitor of COX-2 (IC₅₀=4 μM). F-APHS inhibits the COX-2 activity in RAW 264.7 macrophages with an IC₅₀ of 2.8 μM. However, it did not inhibit the COX-1 activity in uninduced macrophages at concentrations up to 32 μM.

### Example 3

### Radioactive Analogs of APHS

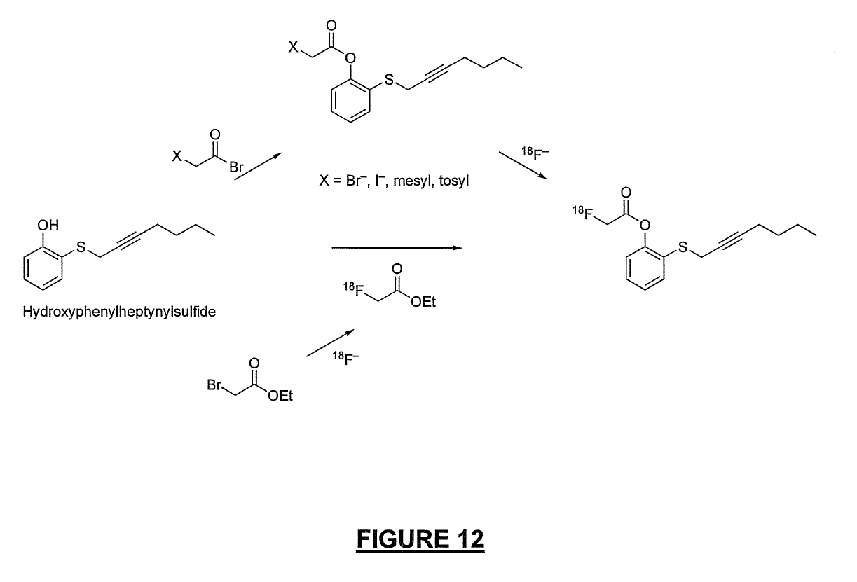

The fluorine atom of F-APHS can also be a radioactive isotope, such as ¹⁸F. A direct synthesis route is a single-step exchange of ¹⁸F⁻ for halogen, mesylate, or tosylate leaving groups. Previous reports indicate that ¹⁸F⁻ exchanges with Br⁻ or I⁻ in bromo- or iodo-acetyl esters or with mesyl or tosyl in mesyl- or tosyl-acetyl esters to form the corresponding ¹⁸F-fluoroacetyl esters without hydrolysis (FIG. 12; Block et al., 1988). The iodo-derivative of APHS has been synthesized, and can be used for the exchange reaction.

Alternatively, an ¹⁸F exchange with the tosyl-derivative of APHS can be used. The latter is available through tosylation of the glycolate ester of APHS. Tosylates are readily exchanged by F⁻, so this method is a facile alternative in the event that exchange with iodo-APHS is undesirable (Block et al., 1988).

One potential complication of the exchange reaction is hydrolysis of the acetyl-phenolate during ¹⁸F exchange. Although this is considered unlikely, an alternative synthesis of ¹⁸F-APHS has been designed in the event it occurs (FIG. 12). Others have reported a two-step synthesis of ¹⁸F-containing compounds in which ¹⁸F⁻ exchange is performed on ethyl-bromoacetate then the ethyl-fluoroacetate is reacted with the nucleophilic center to be acylated (Tada et al., 1990; Jalilian et al., 2000). This two-step scheme has been used to make ¹⁸F-fluoroacetyl amides and esters.

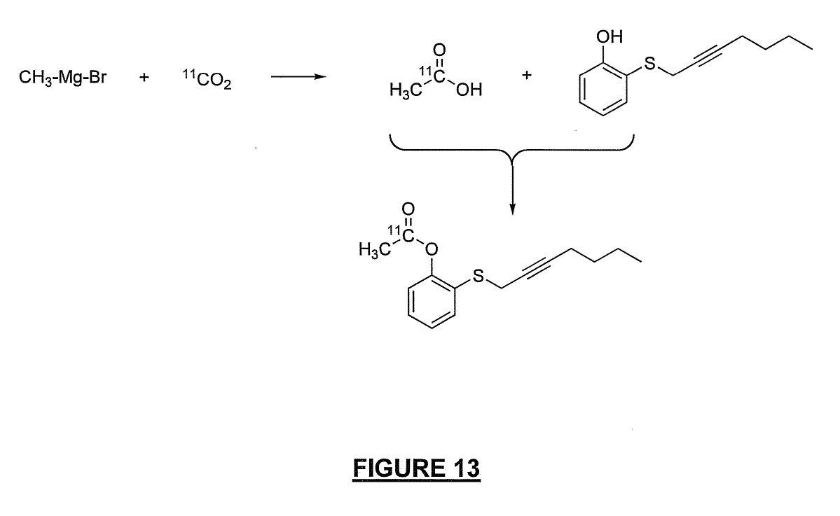

An alternate strategy for covalent imaging of COX-2 is to synthesize APHS labeled with ¹¹C in the acetyl group (FIG. 13). Procedures have been described in which ¹¹CO₂ is converted to ¹¹C-sodium acetate, which is rapidly purified by chromatography and solvent evaporation (Ishiwata et al., 1995; van den Hoff et al., 2001). The purified material is protonated and reacted with an excess of hydroxyphenylheptynylsulfide to directly produce ¹¹C-APHS. APHS is much less polar than either acetic acid or hydroxyphenylheptynylsulfide., so ¹¹C-APHS is purified by passage through a straight phase silica-based SEP-PAK™ matrix (Waters Corp., Milford, Mass., United States of America). The ¹¹C-APHS elutes first from the column. The acetylation of hydroxyphenylheptynylsulfide is rapid as are the manipulations necessary for workup and purification.

### Example 4

### COX-2-Selective NSAID Derivatives as In Vivo Imaging Agents

### Fluorescent Derivatives

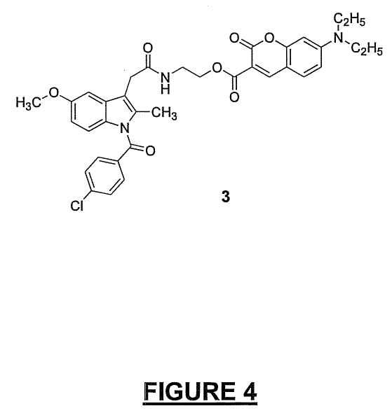

Compound 3, a coumarin-derived ester of the ethanolamide of indomethacin (see FIG. 4) was synthesized according to the method of Timofeevski et al. (2002). This compound is very weakly fluorescent in buffer but yields a strong fluorescent signal on binding to COX-2. The signal is comprised of two components, a non-selective component exhibited on binding to both COX-1 and COX-2, and a selective component that is only observed with COX-2. The kinetics of the specific fluorescence increase corresponds exactly to those of the inhibition of COX-2 by the agent. Compound 3 binds to both apo- and holo-COX-2 but a COX-2-selective fluorescence increase is only observed with apo-protein. The heme prosthetic group of the holo-enzyme quenches the fluorescence.

While compound 3 would not be expected to be a highly successful imaging agent in vivo due to interference from hemoglobin in surrounding tissue, results obtained from these tests are useful in the construction of other fluorescent COX-2-selective optical imaging agents. These agents bind to holo-enzyme without loss in fluorescence, and exhibit minimal interference from hemoglobin or water allowing their use in cells and tissues. The selection of fluorophores having absorption and emission maxima at wavelengths in the near infrared (NIR) is ideal for this purpose, as these wavelengths fall between the absorption spectra of heme and water (Weissleder 2001).

Fluorinated indomethacin and ketorolac derivatives have been synthesized that are potent and highly selective COX-2 inhibitors. The p-fluorophenyl derivative of indomethacin amide (Compound 18) and the p-fluorobenzyl derivative of ketorolac amide (Compound 19) exhibit IC₅₀ values of 52 nM and 80 nM, against purified COX-2, respectively. Compound 18 exhibits anti-inflammatory activity in the rat footpad edema assay following oral installation. Its bioavailability is 30% at a dose of 2 mg/kg and it has a 4 hr half-life in plasma following oral administration. Compound 19 has been shown that it is active in intact cells, inhibiting PGD₂ synthesis by LPS-activated RAW 264.7 cells with an IC₅₀ of 200 nM.

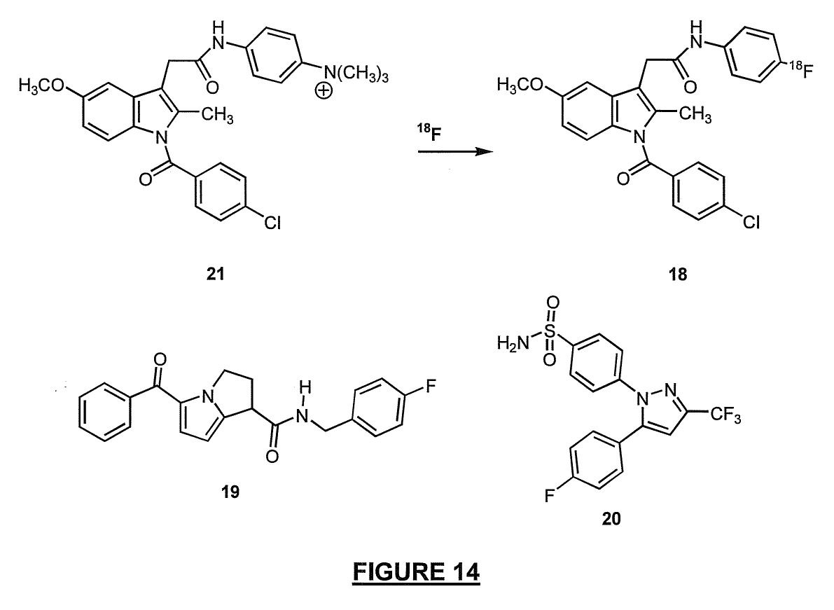

Compounds 18 and 19 are synthesized with ¹⁸F for PET imaging. In both cases, standard chemistry is employed in which p-trimethylammonium precursors are synthesized then exchanged with ¹⁸F⁻ (FIG. 14). Similar chemistry has been reported by McCarthy et al. for the synthesis of an ¹⁸F-labeled COX-2 inhibitor of the diarylheterocycle class (Compound 20) (McCarthy et al., 2002). Compound 20 contains a p-methoxyphenyl group and a pyrazole group, which are similar to the p-methoxyindole group and the pyrrole group in 18 and 19. ¹⁸F⁻ exchange has been successfully reported for compounds containing simple carboxylic acid esters, which are of comparable hydrolytic stability to the p-chlorobenzoyl group of 21. Hydrolysis of the p-chlorobenzoyl group of 21 is also carried out.

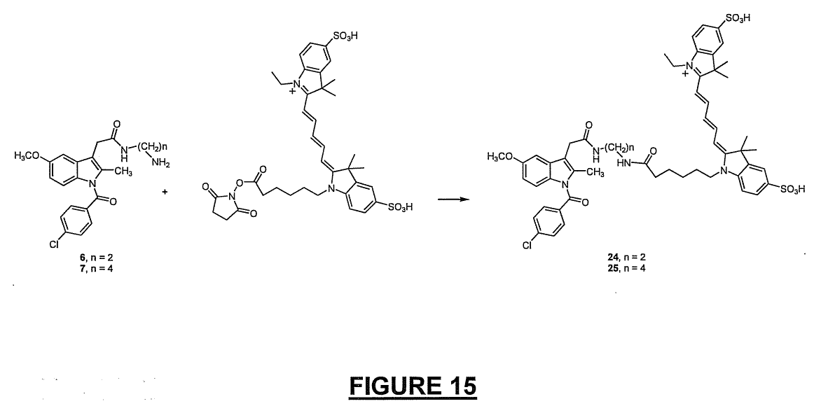

Fluorescent COX-2 inhibitors are also synthesized by coupling indomethacin to commercially available NIR fluorophores such as the succinimide esters Cy5, Cy5.5, and Cy7, supplied by Amersham Biosciences. The availability of the compounds with an activated carboxyl group provides an easy synthetic route to the desired inhibitors, by using indomethacin containing an amine linker. The structures of Cy5-indomethacin conjugates (Compounds 24 and 25) are shown in FIG. 15. The absorption and emission maxima of Cy5 are 650 nm and 668 nm, respectively. Cy5.5 and Cy7 have maxima at longer wavelengths. Molecular Probes also offers a series of NIR fluorophores available as succinimide esters. These compounds, Alexa 647, 660, 680, 700, and 750, have absorption and emission maxima that range from 650 nm to 780 nm, thus encompassing the entire NIR spectrum. They also offer higher extinction coefficients and greater stabilities than the Cy series of dyes.

### Example 5

### COX-2-Selective NSAID Derivatives as In Vivo Imaging Agents

### Iodine-Containing Agents

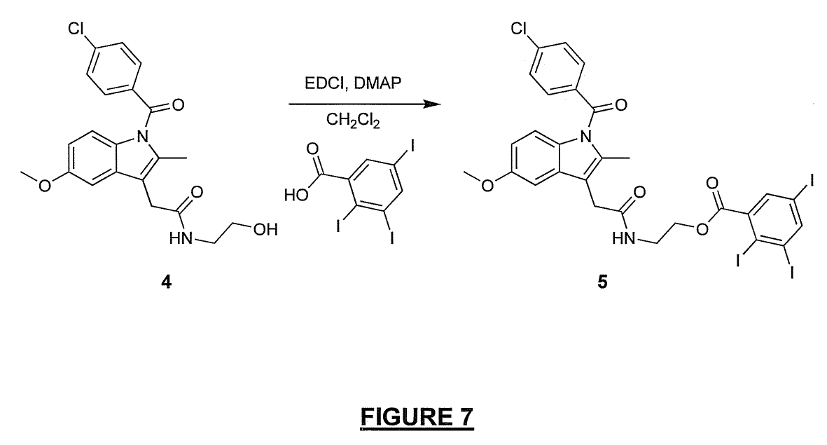

Several approaches have been used to synthesize iodine-containing X-ray contrast agents. The esterification of the ethanolamide of indomethacin has been accomplished by carbodiimide coupling of indomethacin ethanolamide (Compound 4) and 2,3,5-triiodobenzoic acid (FIG. 7). The product, Compound 5, is a potent and highly selective COX-2 inhibitor (IC₅₀ for COX-2=50 nM, IC₅₀ for COX-1>50 μM). Higher concentrations are required for inhibition of COX-2 in the RAW264.7 macrophage cell line (IC₅₀=3.5 μM), which might be related to the hydrophobicity of the compound (cLogP=8.5). Amide derivatives (Compounds 8 and 9) that correspond to the ester, Compound 5, are generated. Compounds 6 and 7 are synthesized and their coupling to 2,3,5-triiodobenzoic acid is carried out

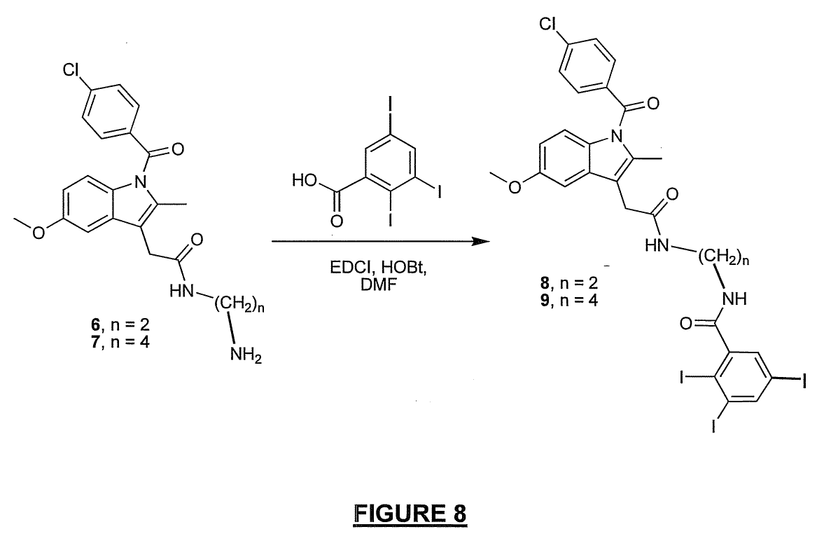

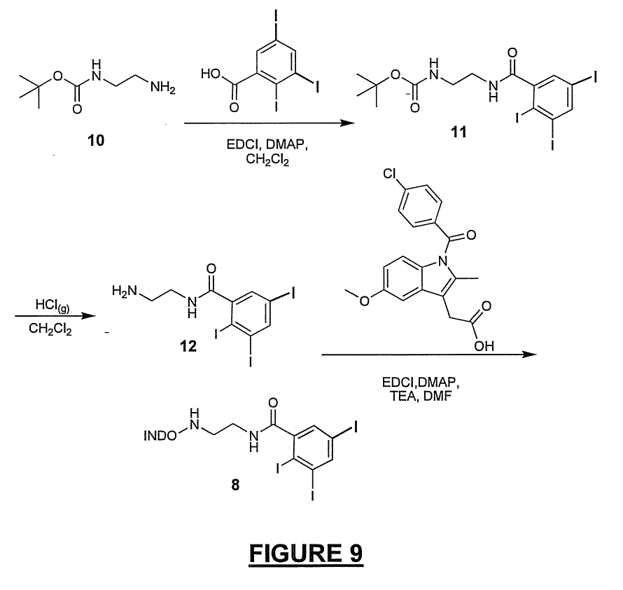

In addition to the straightforward coupling outlined in FIG. 8, the alternate strategy outlined in FIG. 9 can also be used to produce an iodine-containing NSAID. The scheme in FIG. 9 has the advantage of generating the nucleophilic primary amine under conditions that do not expose the base-labile p-chlorobenzoyl group of the indomethacin moiety to strong base.

### Example 6

### COX-2-Specific NSAID Derivatives as In Vivo Imaging Agents

### Chelating Agents

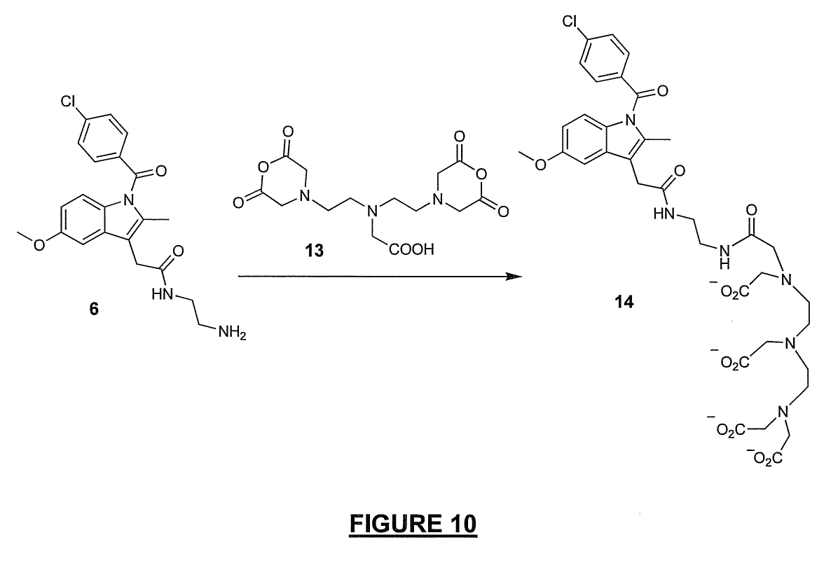

Radiological and/or optical imaging agents comprising heavy metal chelating derivatives of NSAIDs are synthesized. The diethyltriaminepentaacetic acid conjugate to Compound 6 as well as its Gd³⁺ derivative, Compound 15, have been synthesized (see FIG. 10). The use of an excess of the DTPA dianhydride, Compound 13, generated the desired product cleanly and efficiently. Purification of the product was accomplished by reverse phase silica gel chromatography. Gd³⁺ was successfully added to the chelator by dissolving the hexahydrate chloride salt in water, and successful incorporation was confirmed by mass spectrometry. The uncomplexed chelator, Compound 14, displayed no inhibitory activity against COX-2 or COX-1 whereas the Gd³⁺ derivative, Compound 15, exhibited weak COX-2 inhibition.

### Materials and Methods for Examples 7-9

All reactions were performed under an atmosphere of ultra high purity argon. Commercially obtained chemicals were used as received. Reactions were monitored using thin layer chromatography (TLC) plates (Silica Gel 60 F₂₅₄ precoated, 20×10 cm, 0.25 mm) from Analtech, Inc. (Newark, Del., United States of America). Purification was performed on column chromatography using silica followed by recrystallization from EtOAc/hexanes. ¹H and ¹³C NMR data were recorded on a Bruker AC-300 NMR System (Bruker Bio-Spin Corp., Billerica, Mass., United States of America) at 300 and 75 MHz, respectively, in CDCl₃ unless otherwise noted. Chemical shifts are reported in parts per million (ppm) downfield from TMS (δ=0); coupling constants are given in hertz. Positive ion channel electrospray ionization (ESI) and collision-induced dissociation (CID) mass spectra were obtained on a Finnigan TSQ 7000 mass spectrometer (Thermo Electron Corp., Waltham, Mass., United States of America).

### Example 7

### Synthesis of Indolyl Amides of Indomethacin

Indolyl amides of indomethacin were synthesized using the general scheme outlined in FIG. 16.

2-[1-(4-Chloro-benzoyl)-5-methoxy-2-methyl-1H-indol-3-yl]-acetamide (Compound 301). Indomethacin (3.5 g, 0.010 mol) and hydroxybenzotriazole (2 g, 0.015 mol) were dissolved in DMF (100 ml). To the mixture was added ammonia in dioxane, 0.5 M (50 ml, 0.025 mol). The mixture was cooled to 0° C. and 1-cyclohexyl-3-(2-morpholino-ethyl)carbodiimide metho-p-toluenesulfonate (5 g, 0.012 mol) was added. The reaction was stirred overnight and allowed to warm to room temperature. All solvents were removed via high vacuum and residue was taken up in ethylacetate (1200 ml) and brine (500 ml). The reaction was partitioned between two 1000 ml Erlenmeyer flasks for ease of handling. Mixtures were heated to completely dissolve all solids. The organic layer was washed with NaOH (1 N, 6×30 mL) to remove all traces of indomethacin. Yield, 95%; ¹H NMR (MeOH-d₄) δ 7.72-7.63 (m, 4H), 7.45 (s, 1H), 7.12 (s, 1H), 6.97-6.91 (m, 2H), 6.72-6.69 (m, 1H), 3.77 (s, 3H), 3.47 (s, 2H), 2.23 (s, 3H); ¹³C NMR (MeOH-d₄) 171.9, 168.2, 155.9, 137.9, 135.5, 134.6, 131.5, 131.3, 130.7, 129.4, 114.9, 111.5, 102.3, 55.8, 31.3, 13.7; ESI-CID 379 (MNa⁺), m/z 298, 89, 23.

5-methoxy-2-methyl-3-indolacetamide (Compound 303). Compound 301 (3.5 g, 9.8 mmol) was dissolved in dry DMF (100 mL) and stirred at room temperature. NaOH (10 N, 20 mL) was slowly added in small quantities over 1 hour while monitoring the reaction by TLC. The reaction was judged complete after 2 hours by TLC. The pH was lowered to 9 by the addition of HCl (4 N). DMF was evaporated via high vacuum rotovap, and syrup was taken up in ethylacetate (600 ml) and washed with sodium bicarbonate (3×300 mL). The aqueous layer was washed with ethylacetate (3×400 ml), and all organic extracts were combined, dried with sodium sulfate, and solvents removed to give 99% product.

2-(5-Methoxy-2-methyl-1H-indol-3-yl)-ethylamine (Compound 268). Compound 303 (273 mg, 0.7 mmol) was dissolved in freshly distilled THF (30 ml) and cooled to 0° C. Slow addition of a 1 M solution of LAH (0.85 ml, 0.85 mmol) was made with vigorous gaseous evolution noted. The reaction was stirred at room temperature (RT) for 6 days (144 hours), after which time it was poured slowly onto ice water and diluted with ether (150 ml). The aqueous layer was washed with ether (2×150 mL) and all ether extracts were combined and acidified with HCl (1 N, 3×150 mL). The acid extracts were treated with 4 N NaOH until pH 10 and the products were extracted into ether (3×150 mL), dried, and concentrated to give selectively 1-(4-bromobenzyl)-5-methoxy-2-methyl-3-indolethylamine in 55% yield. No 1-benzyl-5-methoxy-2-methyl-3-indolethylamine was detected.

[2-(5-Methoxy-2-methyl-1H-indol-3-yl)-ethyl]-carbamic acid tert-butyl ester (Compound 277). Compound 268 (50 mg, 0.25 mmol) was stirred while dicarbonate (64 mg, 0.29 mmol) in DMF (50 μL) was added at 23° C. The reaction stirred for 18 hours and was judged complete by TLC. The reaction was concentrated to a syrup and dissolved in EtOAc (5 mL), washed with saturated sodium bicarbonate (2×2 mL), dried with sodium sulfate, and concentrated to give product (74 mg; 99%) which was used immediately in the next step.

{2-[1-(4-Bromo-benzyl)-5-methoxy-2-methyl-1H-indol-3-yl]-ethyl}-carbamic acid tert-butyl ester (Compound 278). NaH (7 mg, 0.29 mmol) was added dropwise to a solution of Compound 277 (74 mg, 0.25 mmol) in DMF (10 mL) at 0° C. The reaction mixture was stirred for 20 minutes at 0° C. at which time bromobenzyl bromide (72 mg, 0.29 mmol) was added. The reaction stirred overnight and was diluted carefully with water, extracted with ether (2×10 mL) and washed with water (2×5 mL), dried with sodium sulfate, concentrated, and purified on silica (EtOAc 10% in hexanes) to give a yellow solid (20 mg, 17%)

2-[1-(4-Bromo-benzyl)-5-methoxy-2-methyl-1H-indol-3-yl]-ethylamine (Compound 279). HCl gas (HCl_{(g)}) was gently bubbled through a 1 mL solution of Compound 278 in CH₂Cl₂ in a 2 mL vial for 1 hour. The reaction was diluted with water and neutralized with 1 N NaOH added dropwise until pH=9. The product was extracted with CHCl₃ (3×3 mL) and dried with sodium sulfate to give a yellow oil (13 mg, 84%).

### Example 8

### Synthesis of Diamide Derivatives of Indomethacin

Diamide derivatives of indomethacin were synthesized following the general scheme outlined in FIG. 17.

(2-{2-[1-(4-Chloro-benzoyl)-5-methoxy-2-methyl-1H-indol-3-yl]-acetylamino}-ethyl)-carbamic acid tert-butyl ester (Compound 365). In an oven dried round bottomed flask equipped with a magnetic stir bar and a rubber septum were placed indomethacin (1 eq) HOBt (1.1 eq) and (1-[(3-dimethylamino)propyl]-3-ethylcarbodiimide (1.1 eq) in anhydrous CH₂CO₂. To this was added a solution of the BOC-protected diamine in CH₂Cl₂. Stirring was continued for 18 hours. The reaction mixture was then quenched by pouring the mixture into a separatory funnel containing aqueous saturated sodium bicarbonate followed by H₂O. The organic layer was collected and dried over anhydrous sodium sulfate. After filtration, the solvent was removed under reduced pressure to give a yellow solid. Purification was performed by flash column chromatography (silica gel, 50% EtOAc in Hexane) to give a white powder (7.7 g, 60%). ¹H NMR (CDCl₃) δ 7.70 (d, J=8.3 Hz, 2H), 7.48 (d, J=8.1 Hz, 2H), 6.91-6.88 (m, 2H), 6.69 (dd, J=2.1, 9.1 Hz, 2H), 6.29 (s, 1H), 3.82 (s, 3H), 3.63 (s, 2H), 3.35-3.29 (m, 2H), 3.21-3.16 (m 2H), 2.38 (s, 3H), 1.35 (s, 9H); ESI 500 (MH⁺)

N-(2-Amino-ethyl)-2-[1-(4-chloro-benzoyl)-5-methoxy-2-methyl-1H-indol-3-yl]-acetamide (Compound 377). The appropriate indo-BOC-aminoamide (1 eq) was dissolved in CH₂Cl₂ in a three neck round bottomed flask fitted with a reflux condenser in the center. A septum was placed in one opening while a second septum with a hole bored into it and containing a glass pasture pipette. The pipette was connected to the HCl gas cylinder via a TEFLON® tube. Gentle bubbling of the gas was maintained for 0.5 hours during which time the reaction develops a precipitate. TLC confirmed the consumption of starting material. The crude reaction was then concentrated in vacuo to give a solid (722 mg, 99%), which was used without further purification. ¹H NMR (CDCl₃) δ 7.66 (d, J=8.3 Hz, 2H), 7.47 (d, J=8.4 Hz, 2H), 6.91-6.88 (m, 3H), 6.69 (dd, J=2.2, 9.0 Hz, 2H), 6.29 (s, 1H), 3.82 (s, 3H), 3.65 (s, 2H), 3.28-3.23 (m, 2H), 2.75 (s, 2H), 2.39 (s, 3H); ¹³C NMR (CDCl₃) δ 170.6, 168.7, 156.6, 139.9, 136.6, 134.0, 131.6, 131.3, 130.8, 129.6, 115.5, 113.4, 112.6, 101.2, 56.1, 42.6, 41.6, 32.7, 28.7, 13.7; ESI 400 (MH⁺).

N-(2-{2-[1-(4-Chloro-benzoyl)-5-methoxy-2-methyl-1H-indol-3-yl]-acetylamino}-ethyl)-4-dimethylamino-benzamide (Compound 354). Compound 351 (210 mg, 0.5 mmol), dimethylaminobenzoic acid (264 mg, 1.5 mmol), EDCl (304 mg, 1.5 mmol), HOBt (215 mg, 1.5 mmol) and DIPEA (87 μL, 1.5 mmol) were dissolved in DMF (dry, 15 mL) and allowed to stir 18 hours. The reaction was quenched with saturated sodium bicarbonate (30 ml) and diluted with CHCl₃ (30 mL). The organic layers were combined, and concentrated and purified on silica gel (25% EtOAc in hexanes) to give a white solid (118 mg, 43%); ¹H NMR (MeOH-d₄) δ 7.68 (d, J=8.5 Hz, 2H), 7.48 (d, J=8.9 Hz, 2H), 7.42 (d, J=8.5 Hz, 2H), 6.84 (d, J=9.2 Hz, 2H), 6.73 (s, 1H), 6.65 (dd, J=2.3, 9.0 Hz, 1H), 6.57 (d, J=8.9 Hz, 1H), 3.75 (s, 3H), 3.61 (s, 2H), 3.44 (s, 4H), 2.34 (s, 3H); ¹³C NMR (MeOH-d₄) δ 171.9, 168.8, 156.6, 139.5, 138.0, 137.0, 134.2, 131.7, 131.4, 130.7, 129.5, 128.8, 121.7, 115.6, 113.0, 112.5, 111.4, 101.1, 56.1, 41.3, 40.5, 32.5, 13.7; ESI-CID 547 (MH⁺) m/z 382, 148.

N-(2-{2-[1-(4-Chloro-benzoyl)-5-methoxy-2-methyl-1H-indol-3-yl]-acetylamino}-ethyl)-4-fluoro-benzamide (Compound 355). Compound 351 (210 mg, 0.5 mmol), fluorobenzoic acid (210 mg, 1.5 mmol), EDCl (304 mg, 1.5 mmol), HOBt (215 mg, 1.5 mmol) and DIPEA (87 μL, 1.5 mmol) were dissolved in DMF (dry, 15 mL) and allowed to stir 18 hours. The reaction was quenched with saturated sodium bicarbonate (30 ml) and diluted with CHCl₃ (30 mL). The organic layers were combined and concentrated and purified on silica gel (25% EtOAc in hexanes) to give a white solid (72 mg, 30%); ¹H NMR (CDCl₃) δ 7.68 (d J=8.6 Hz, 2H), 7.65-7.62 (m, 1H). 7.57, (d, J=8.6 Hz, 1H), 7.45 (d, J=8.5 Hz, 2H), 7.32 (d, J=8.6 Hz, 1H), 7.23-7.10 (m, 1H), 7.04 (t, J=8.6 Hz, 2H), 6.86-6.82 (m, 2H), 6.65 (dd, J=2.4, 9.1 Hz, 1H), 6.56-6.50 (m, 1H), 3.74 (s, 3H), 3.63 (s, 2H), 3.50-3.40 (m, 4H), 2.34 (s, 3H); ¹³C NMR (CDCl₃) δ 172.5, 168.7, 167.4, 156.6, 139.9, 137.0, 134.0, 131.6, 130.7, 129.7, 129.6, 129.2, 128.8, 116.1, 115.8, 115.6, 112.7, 112.4, 101.2, 56.01, 41.5, 40.7, 32.5, 13.7; ESI-CID 522 (MH⁺), m/z 312, 245, 174.

N-(2-{2-[1-(4-Chloro-benzoyl)-5-methoxy-2-methyl-1H-indol-3-yl]-acetylamino}-ethyl)-4-trimethylamoniumtrifluoromethanesulfonyl-benzamide (Compound 361). Compound 354 (31.2 mg, 0.057 mmol) was dissolved in CH₂Cl₂ (dry, 20 mL) and methoxy trifluoromethane sulfonate (7.5 μL, 0.068 mmol) was added dropwise. The reaction was stirred for 18 hours, after which another aliquot of the triflate (20 μL) was added. The reaction was stirred for another 18 hours, at which time ether was added (5 mL) to produce a slight precipitate. Distilled water (20 mL) was added to dissolve the precipitate and the aqueous layer was collected and concentrated to give a green oil (25 mg, 62%); ¹H NMR (MeOH-d₄) δ 7.78 (d, J=9.2 Hz, 2H), 7.72 (d, J=9.1 Hz, 2H), 7.62 (d, J=8.4 Hz, 2H), 7.46 (d, J=8.4 Hz, 2H), 6.91-6.86 (m, 2H), 6.53 (dd, J=2.3, 9.1 Hz, 1H), 3.66 (s, 3H), 3.59 (s, 9H), 3.42-3.38 (m, 4H), 2.15 (s, 3H); ¹³C NMR (MeOH-d₄) δ ESI-CID 561 (MH⁺), m/z 312, 148.

N-(2-{2-[1-(4-Chloro-benzoyl)-5-methoxy-2-methyl-1H-indol-3-yl]-acetylamino}-ethyl)-4-hydroxy-benzamide (Compound 380). Compound 377 (106 mg, 0.27 mmol), EDCl (76 mg, 0.40 mmol), DIPEA (70 μL, 0.40 mmol) p-hydroxybenzoic acid (55 mg, 0.40 mmol) and HOBt (54 mg, 0.40 mmol) were dissolved in DMF (dry, 20 mL) and allowed to stir for 36 hours at room temperature. The reaction was quenched with saturated sodium bicarbonate (3×30 mL) and diluted with EtOAc (30 mL). The organic layer was concentrated in vacuo and purified on silica (EtOAc 80% in hexanes) to give a white solid, which was recrystallized from EtOAc (52 mg, 38%); ¹H NMR 400 MHz (DMSO-d₆) δ 9.92 (s, 1H), 8.18 (t, J=5.1 Hz, 1H), 8.07 (t, J=5.0 Hz, 1H), 7.67 (d, J=8.6 Hz, 2H), 7.64-7.61 (m, 4H), 7.08 (d, J=2.3 Hz, 1H), 6.93 (d, J=9.0 Hz, 1H), 6.74 (d, J=8.6 Hz, 2H), 6.69 (dd, J=2.4, 9.0 Hz, 1H), 3.73 (s, 3H), 3.49 (s, 2H), 2.05-1.77 (m, 4H), 2.49 (s, 3H); ¹³C NMR 400 MHz (DMSO-d₆) δ 170.1, 168.2, 166.6, 160.5, 155.9, 137.9, 136.0, 135.6, 135.0, 134.6, 131.5, 131.3, 130.7, 129.4, 115.1, 114.9, 114.5, 111.6, 102.1, 55.8, 31.6, 13.7; ESI 520 (MH⁺).

N-(2-{2-[1-(4-Chloro-benzoyl)-5-methoxy-2-methyl-1H-indol-3-yl]-acetylamino}-ethyl)-4-iodo-benzamide (Compound 381). Compound 377 (109 mg, 0.27 mmol), EDCl (78 mg, 0.41 mmol), DIPEA (71 μL, 0.41 mmol), p-iodobenzoic acid (102 mg, 0.41 mmol), and HOBt (55 mg, 0.41 mmol) were dissolved in DMF (dry, 20 mL) and allowed to stir for 36 hours at room temperature. The reaction was quenched with saturated sodium bicarbonate (3×30 mL) and diluted with EtOAc (30 mL). The organic layer was concentrated in vacuo and purified on silica (EtOAc 80% in hexanes) to give a white solid, which was recrystallized from EtOAc (88.4 mg, 52%); ¹H NMR 400 MHz (DMSO-d₆) δ 7.64 (m, 1H), 7.25 (m, 1H), 6.94 (d, J=8.1 Hz, 1H), 6.82 (d, J=8.6 Hz, 2H), 6.77 (d J=8.3 Hz, 2H), 6.67 (d, J=8.1 Hz, 2H), 6.24 (s, 1H), 6.08 (d, J=9.0 Hz, 1H), 5.83 (dd, J=2.2, 8.9 Hz, 1H), 2.88 (s, 3H), 2.65 (s, 2H), 2.42-2.38 (m, 4H), 1.34 (s, 3H); ¹³C NMR 400 MHz (DMSO-d₆) δ 170.2, 168.2, 166.2, 155.9, 137.9, 137.4, 135.6, 134.6, 134.2, 131.5, 131.2, 130.7, 129.5, 129.4, 114.9, 114.5, 111.6, 102.2, 99.1, 55.7, 31.6, 13.7; ESI 630 (MH⁺).

N-(2-{2-[1-(4-Chloro-benzoyl)-5-methoxy-2-methyl-1H-indol-3-yl]-acetylamino}-ethyl)-4-nitro-benzamide (Compound 382). Compound 377 (117 mg, 0.29 mmol), EDCl (84 mg, 0.44 mmol), DIPEA (77 μL, 0.44 mmol), p-nitrobenzoic acid (74 mg, 0.44 mmol), and HOBt (59 mg, 0.44 mmol) were dissolved in DMF (dry, 20 mL) and allowed to stir for 36 hours at room temperature. The reaction was quenched with saturated sodium bicarbonate (3×30 mL) and diluted with EtOAc (30 mL). The organic layer was concentrated in vacuo and purified on silica (EtOAc 80% in hexanes) to give a white solid, which was recrystallized from EtOAc (107 mg, 67%); ¹H NMR 400 MHz (DMSO-d₆) δ 7.86 (d, J=5.4 Hz, 1H), 7.37 (d, J=8.8 Hz, 2H), 7.24 (d, J=5.2 Hz, 1H), 7.09 (d, J=6.9 Hz, 2H), 6.81 (d, J=8.4 Hz, 2H), 6.76 (d, J=8.6 Hz, 2H), 6.23 (d, J=2.4 Hz, 1H), 6.05 (d, J=9.0 Hz, 1H), 5.81 (dd, J=2.5, 9.0 Hz, 1H), 2.87 (s, 3H), 2.65 (s, 2H), 2.43 (m, 4H), 1.33 (s, 3H); ¹³C NMR (DMSO-d₆) δ 170.2, 168.2, 165.2, 155.9, 149.2, 140.4, 137.9, 135.6, 134.6, 131.5, 131.2, 130.7, 129.4, 129.0, 123.7, 114.9, 114.5, 102.2, 55.7, 31.6, 13.7; ESI-CID 549 (MH⁺).

Toluene-4-sulfonic acid 4-(2-{2-[1-(4-chloro-benzoyl)-5-methoxy-2-methyl-1H-indol-3-yl]-acetylamino}-ethylcarbamoyl)-phenyl ester (Compound 387). Compound 380 (14.5 mg, 0.028 mmol) was dissolved in DMF (2 mL) with pyridine (2 drops). Tosyl chloride (6 mg, 0.031 mmol) was added and the reaction vessel was purged with argon and stirred at room temperature for 15 hours. The reaction was quenched with saturated sodium bicarbonate (2×10 mL) and extracted into CH₂Cl₂ (2×20 mL). The combined organic solution was washed with water (2×20 mL), dried with sodium sulfate, concentrated, and purified on silica (EtOAc 50% in hexanes) to give a yellow solid (6.3 mg, 33%); ¹H NMR (MeOH-d₄) δ 7.60 (d, J=8.1 Hz, 4H), 7.48-7.43 (m, 4H), 7.30 (d, J=8.0 Hz, 2H), 6.88-6.85 (m, 3H), 6.78 (d, J=9.0 Hz, 1H), 6.50 (dd, J=9.0, 2.4 Hz, 1H), 3.65 (s, 3H), 3.51 (s, 2H), 3.23 (m, 4H), 2.34 (s, 3H), 2.17 (s, 3H); ¹³C NMR (MeOH-d₄) δ 174.4, 170.4, 169.5, 158.0, 153.7, 147.9, 140.5, 137.7, 136.1, 134.7, 133.8, 132.8, 132.7, 132.5, 131.6, 130.6, 130.4, 130.1, 123.8, 116.4, 114.9, 113.1, 102.7, 56.5, 41.2, 41.0, 32.8, 22.1, 14.0;

### Example 9

### Synthesis of Amide Derivatives of Indomethacin

Amide derivatives of indomethacin were synthesized using the general scheme outlined in FIG. 18.

N-(4-Fluoro-phenyl)-2-(5-methoxy-2-methyl-1H-indol-3-yl)-acetamide (Compound 375). Method A. To a solution of 5-methoxy-2-methyl-1H-indoleacetic acid (1 g, 4.6 mmol) in dry CH₂Cl₂ (30 mL) was added DMAP (0.83 g, 6.8 mmol) and EDCl (1.3 g, 6.8 mmol) followed by 4-fluoroaniline (0.65 mL, 6.8 mmol). The reaction was allowed to stir for 18 hours at 23° C. The mixture was diluted with water (30 mL) and extracted with EtOAc (2×30 mL). The combined organic extracts were washed with water (2×30 mL), dried with sodium sulfate, concentrated, and purified on silica (20% EtOAc in hexanes) to give a white powder (433 mg, 30%).

Method B. Compound 360 (341 mg, 0.76 mmol; see FIG. 18) was dissolved in dry DMF (20 mL) and 10 N NaOH (513 μL) was added portionwise over 3 hours. The reaction was judged complete by TLC and quenched with water (100 mL) and extracted with EtOAc (2×50 mL). The combined organic layers were washed with water (2×30 mL) and dried (MgSO₄) to give a white powder (203 mg, 86%), which was used without further purification. ¹H NMR 400 MHz (CDCl₃) δ 8.04 (s, 1H), 7.37 (s, 1H), 7.31-7.24 (m, 3H), 6.95-6.90 (m, 3H), 6.83 (dd, J=2.4, 8.7 Hz, 1H), 3.81 (s, 3H), 3.78 (s, 2H), 2.42 (s, 3H)

2-[1-(4-Chloro-2-fluoro-benzoyl)-5-methoxy-2-methyl-1H-indol-3-yl]-N-(4-fluoro-phenyl)-acetamide (Compound 360). Compound 375 (57 mg, 0.18 mmol) was dissolved in dry DMF (10 mL) and cooled to 0° C. NaH (8.7 mg, 0.36 mmol) was added portionwise and the reaction was stirred for 20 minutes. To the reaction was added 2-fluoro-4-chloro-benzoyl chloride (70 mg, 0.36 mmol). The mixture was allowed to stir at 23° C. for 17 hours at which time TLC showed ˜50% conversion of starting material. Another 70 mg of the benzoyl chloride followed by 15 mg NaH was added to the reaction and allowed to stir for an additional 18 hours. The reaction was poured carefully onto ice water (20 mL) and extracted with EtOAc (2×30 mL). The combined organic layers were washed with 10% HCl (2×10 mL), dried with sodium sulfate, purified on silica (10% EtOAc in hexanes) to give yellow solid (28 mg, 33%); ¹H NMR (CDCl₃) δ 7.94 (t, J=9.0 Hz, 1H), 7.59 (t, J=8.1 Hz, 1H), 7.34-7.30 (m, 3H), 7.30-7.17 (m, 2H), 6.96 (d, J=8.9 Hz, 1H), 6.91 (d, J=1.2 Hz, 1H), 6.76 (dd, J=2.5, 9.0 Hz, 1H), 3.80 (s, 3H), 3.77 (s, 2H), 2.36 (s, 3H); ¹³C NMR (CDCl₃) δ 169.0, 163.0, 156.4, 135.7, 135.2, 133.7, 131.69, 130.1, 126.2, 125.3, 121.3, 120.5, 118.1, 117.8, 117.5, 115.8, 115.5, 115.1, 111.9, 102.7, 55.8, 32.2, 13.7; ESI 491 (MNa⁺).

4-Chloro-2-nitro-benzoyl chloride (Compound 384). A mixture of 4-Chloro-2-nitro-benzoic acid (2 g, 9.9 mmol) and SOCl₂ (8.5 mL, 114.8 mmol) and DMF (66 μL) was stirred at 26° C. for 4 hours. When evolution of HCl subsided the temperature was raised to 65° C. with stirring for 1 hours. After removal of excess SOCl₂ by vacuum distillation, the residue was dissolved in 1,2 dichloromethane (2 mL) and evaporated. The residue was dissolved in 10 mL of 1,2 dichloromethane and treated twice with decolorizing charcoal and filtered to give the final product in quantitative yield which was used without further purification. ¹H NMR (CDCl₃) δ 7.95 (s, 1H), 7.74 (s, 2H).

2-[1-(4-Chloro-2-nitro-benzoyl)-5-methoxy-2-methyl-1H-indol-3-yl]-N-(4-fluoro-phenyl)-acetamide (Compound 385). Compound 375 (100 mg, 0.32 mmol) was dissolved in DMF (dry, 5 mL) and cooled to 0° C. NaH (7.7 mg, 0.32 mmol) was added portionwise and the reaction was allowed to stir for 20 minutes. A clear to yellow color change was noted. Compound 384 (100 μL, 0.48 mmol) was added dropwise with an immediate color change to orange. The reaction stirred for 18 hours and was allowed to warm to room temperature. The reaction was diluted in CH₂CL₂ (30 mL) and quenched with 10% HCl (30 mL) solution. The organic layer was concentrated and purified on silica gel (EtOAc, 20% in hexanes) to give a brown syrup (51 mg, 32%); ¹H NMR (CDCl₃) δ 8.15 (d, J=1.9 Hz, 1H), 7.81 (dd J=1.9, 8.2 Hz, 1H), 7.62 (d, J=8.2 Hz, 1H), 7.40-7.36 (m, 2H), 7.28 (s, 1H), 6.97-6.84 (m, 4H), 6.68 (dd, J=2.3, 9.0 Hz, 1H), 3.79 (s, 3H), 3.74 (s, 2H), 2.39 (s, 3H); ¹³C NMR (CDCl₃) δ 168.5, 164.4, 156.6, 146.8, 136.5, 135.8, 135.4, 135.2, 131.9, 131.2, 131.0, 130.1, 125.8, 121.4, 121.3, 116.0, 115.8, 115.5, 112.0, 103.0, 55.8, 32.5, 14.0; ESI 471 (MH⁺).

2-[1-(4-Bromo-benzyl)-5-methoxy-2-methyl-1H-indol-3-yl]-ethylamine (Compound 388). Compound 277 (136 mg, 0.29 mmol) was dissolved in CH₂Cl₂ (dry, 6 mL) and HCl_{(g)} was bubbled gently through mixture until TLC indicated complete consumption of starting material. Saturated sodium bicarbonate (15 mL) was slowly added to neutralize mixture which was extracted with CH₂Cl₂ (2×20 mL). The combined organic solution was washed with water (2×20 mL), dried with sodium sulfate, and concentrated to give the product in quantitative yield (107 mg, 100%), which was used without further purification.

N-{2-[1-(4-Bromo-benzyl)-5-methoxy-2-methyl-1H-indol-3-yl]-ethyl}-4-nitro-benzamide (Compound 389). Compound 388 (42 mg, 0.11 mmol), EDCl (25 mg, 0.13 mmol), DIPEA (23 μL, 0.13 mmol) p-nitrobenzoic acid (22 mg, 0.13 mmol), and HOBt (18 mg, 0.13 mmol) were dissolved in DMF (dry, 5 mL) and allowed to stir for 18 hours at room temperature. The reaction was quenched with saturated sodium bicarbonate (2×10 mL) and extracted with EtOAc (2×20 mL). The combined organic solution was washed with water (2×20 mL), dried with sodium sulfate, concentrated, and purified on silica (EtOAc 50% in hexanes) to give a yellow solid (11 mg, 20%); ¹H NMR (CDCl₃) δ 8.18 (d, J=8.8 Hz, 2H), 7.71 (d, J=8.8 Hz, 2H), 7.34 (d, J=8.4 Hz, 2H), 7.08 (d, J=8.8 Hz, 2H), 7.00 (d, J=2.3 Hz, 1H), 6.81-6.76 (m, 3H), 6.20 (s, 1H), 5.20 (s, 2H), 3.78 (s, 3H), 3.72-3.71 (m, 2H), 3.08-3.06 (m, 2H), 2.24 (s, 3H); ¹³C NMR (CDCl₃) δ 154.7, 137.3, 132.3, 128.3, 128.0, 124.2, 111.4, 110.4, 100.7, 56.31, 46.61, 24.53, 10.76; ESI-CID 522 (MH⁺).

N-{2-[1-(4-Bromo-benzyl)-5-methoxy-2-methyl-1H-indol-3-yl]-ethyl}-4-fluoro-benzamide (Compound 390). Compound 388 (42 mg, 0.11 mmol), EDCl (25 mg, 0.13 mmol), DIPEA (23 μL, 0.13 mmol) p-fluorobenzoic acid (18 mg, 0.13 mmol), and HOBt (18 mg, 0.13 mmol) were dissolved in DMF (dry, 5 mL) and allowed to stir for 18 hours at room temperature. The reaction was quenched with saturated sodium bicarbonate (2×10 mL) and extracted with EtOAc (2×20 mL). The combined organic solution was washed with water (2×20 mL), dried with sodium sulfate, concentrated, and purified on silica (EtOAc 50% in hexanes) to give a yellow solid (30 mg, 56%); ¹H NMR (CDCl₃) δδ 8.11-8.08 (m, 1H), 7.61-7.56 (m, 2H), 7.34 (d, J=8.4 Hz, 2H), 7.16-6.98 (m, 5H), 6.77 (d, J=8.5 Hz, 2H), 5.19 (s, 2H), 3.76 (s, 3H), 3.69-3.67 (m, 2H), 3.06-3.01 (m, 2H), 2.23 (s, 3H); ¹³C NMR (CDCl₃) δ 154.7, 137.4, 132.3, 129.5, 129.4, 128.6, 128.1, 116.1, 115.9, 111.4, 110.3, 108.9, 100.7, 56.3, 51.3, 46.6, 41.0, 31.3, 24.7, 10.7; ESI-CID 595 (MH⁺), m/z 356.1, 194.5.

[1-(4-Chloro-2-nitro-benzoyl)-5-methoxy-2-methyl-1H-indol-3-yl]-acetic acid (Compound 391). 5-methoxy-2-methyl-1H-indol-3-yl]-acetic acid (315 mg, 1.43 mmol) was dissolved in DMF (dry, 5 mL) and cooled to 0° C. NaH (69 mg, 2.88 mmol) was added portionwise and the reaction was allowed to stir for 20 minutes. 4-Chloro-2-fluoro-benzoyl chloride (275 μL, 2.15 mmol) was added. The reaction was allowed to stir for 18 hours and allowed to warm to room temperature. The reaction was quenched with 10% HCl (50 mL) and extracted in CH₂CL₂ (50 mL). The combined organic solution was washed with water (2×20 mL), dried with sodium sulfate, concentrated, and purified on silica gel (EtOAc, 25% in hexanes) to give a brown solid.

### Example 10

### Radiolabeling of Indomethacin Derivatives

The production of ¹⁸F and the exchange chemistry is shown in Scheme 4 (see FIG. 19). The fluorine-18 anion was prepared from ¹⁸O-water using the 12 MeV cyclotron at the Vanderbilt Medical Center Nuclear PET facility (Vanderbilt University, Nashville, Tenn., United States of America). The fluorine-18 anion was then trapped onto an anion exchange column, and eluted with potassium carbonate to give K¹⁸F. The ion pair was delivered to the reaction vessel and complexed with KRYPTOFIX_{2,2,2}® to generate the [KRYPTOFIX_{2,2,2}®-K⁺] [F⁻] ion complex. Upon drying the salt down, substrate (dissolved in 5 mL acetonitrile) was delivered to the reaction vessel and the temperature was brought to 85° C. The reaction was allowed to stand for 30 minutes and then removed from the exchange apparatus for workup and radio-TLC quantification.

### Materials and Methods for Examples 11-12

Enzymology. Arachidonic acid was purchased from Nu Chek Prep (Elysian, Minn., United States of America). [1-¹⁴C]Arachidonic acid (˜55-57 mCi/mmol) was purchased from NEN Dupont (Boston, Mass., United States of America) or American Radiolabeled Chemicals, Inc. (St. Louis, Mo., United States of America). COX-1 was purified from ovine seminal vesicles (Oxford Biomedical Research, Inc., Oxford, Mich., United States of America) as described in Marnett et al., 1984. The specific activity of the protein was 20 (μM O₂/min)/mg, and the percentage of holoprotein was 13.5%. ApoCOX-1 was prepared by reconstitution by the addition of hematin to the assay mixtures as described in Odenwaller et al., 1990. Apoenzyme was reconstituted by the addition of hematin to the assay mixtures. Human COX-2 was expressed in Sf9 insect cells by means of the pVL 1393 expression vector (BD Biosciences Pharmingen, San Diego, Calif., United States of America) and purified by ion exchange and gel filtration chromatography. All of the purified proteins were shown by densitometric scanning of a 7.5% SDS-PAGE gel to be >80% pure.

Time- and Concentration-Dependent Inhibition of Ovine COX-1 and Human COX-2 Using Thin Layer Chromatography (TLC) Assay. Cyclooxygenase activity of ovine COX-1 (44 nM) or human COX-2 (66 nM) was assayed by TLC. Reaction mixtures of 200 μL consisted of hematin-reconstituted protein in 100 mM Tris-HCl, pH 8.0, 500 μM phenol, and [1-¹⁴C]arachidonic acid (50 μM) for 30 seconds at 37° C. Reactions were terminated by solvent extraction in Et₂O/CH₃OH/1 M citrate, pH 4.0 (30:4:1). The phases were separated by centrifugation at 2000 g for 2 minutes and the organic phase was spotted on a TLC plate (J. T. Baker, Phillipsburg, N.J., United States of America). The plate was developed in EtOAc/CH₂CL₂/glacial AcOH (75:25:1)) at 4° C. Radiolabeled prostanoid products observed at different inhibitor concentrations was divided by the percentage of products observed for protein samples preincubated for the same time with DMSO.

Inhibition of COX-2 Activity in Activated RAW264.7. Protocols for COX-2 inhibition in RAW264.7 cells have been previously described (Kalgutkar et al., 1998b). Briefly, cells (6.2×10⁶ cells/T25 flask) were activated with lipopolysaccharide (1 μg/mL) and γ-interferon (10 U/mL) in serum-free DMEM for 7 hours and then treated with inhibitor (0-2 μM) for 30 minutes at 37° C. Exogenous arachidonate metabolism was determined by adding [1-¹⁴C]-arachidonate acid (20 μM) for 15 minutes at 37° C. IC₅₀ values are the average of two independent determinations.

### Example 11

### Selective COX-2 Inhibition in Purified Enzyme

IC₅₀ values for the inhibition of purified human COX-2 or ovine COX-1 by test compounds were determined by thin layer chromatography (TLC) radiography. Hematin-reconstituted COX-2 (66 nM) or COX-1 (44 nM) in 100 mM Tris-HCl, pH 8.0 containing 500 μM phenol was treated with several concentrations of inhibitors (0-2850 nM) at 25° C. for 20 minutes. The cyclooxygenase reaction was initiated by the addition of [1-¹⁴C]-arachidonic acid (50 μM) at 37° C. for 30 seconds. As indicated in Tables 1-3 below, the fluorinated standards Compounds 355, 360, and 389 displayed potent and selective inhibition of COX-2 over COX-1 with IC₅₀ values in the 50-100 nM range.

### Example 12

### Selective COX-2 Inhibition in RAW264.7 Murine Macrophages

The ability of the fluorinated amide analogs of indomethacin to inhibit COX-2 in intact cells was assayed in RAW264.7 macrophages in which COX-2 activity was induced by pathologic stimuli. The macrophages were exposed to lipopolysaccharide and γ-interferon to induce COX-2 and then treated with several concentrations of Compound 355. The IC₅₀ value for inhibition of prostaglandin D2 production by Compound 355 was 500 nM.

### Discussion of Examples 7-12

Three representative COX-2 selective indomethacin analog precursors for positron emitting tomography (PET) were designed and prepared to investigate the feasibility of a COX-2 selective tumor imaging agent. A fluorinated amide, an indolyl amide, and a diamide analog of indomethacin have been shown to exhibit potent and selective activity against COX-2 in vitro over COX-1 in assays (COX-1 IC₅₀>60 μm for all, COX-2 IC₅₀=50-100 nm). The synthesis of 2-[1-(4-Chloro-2-fluoro-benzoyl)-5-methoxy-2-methyl-1H-indol-3-yl]-N-(4-fluoro-phenyl)-acetamide (Compound 360), N-{2-[1-(4-Bromo-benzyl)-5-methoxy-2-methyl-1H-indol-3-yl]-ethyl}-4-fluoro-benzamide (Compound 390) and N-(2-{2-[1-(4-Chloro-benzoyl)-5-methoxy-2-methyl-1H-indol-3-yl]-acetylamino}-ethyl)-4-fluoro-benzamide (Compound 382) were all carried out using EDCl amide coupling to give 33%, 43% and 56% yields respectively from the appropriate amide precursors. The nitro benzamide analogs were prepared similarly to give 2-[1-(4-Chloro-2-nitro-benzoyl)-5-methoxy-2-methyl-1H-indol-3-yl]-N-(4-fluoro-phenyl)-acetamide (Compound 360), 32%; N-(2-{2-[1-(4-Chloro-benzoyl)-5-methoxy-2-methyl-1H-indol-3-yl]-acetylamino}-ethyl)-4-nitro-benzamide (Compound 382), 67%; and N-{2-[1-(4-Bromo-benzyl)-5-methoxy-2-methyl-1H-indol-3-yl]-ethyl}-4-fluoro-benzamide (Compound 390), 56%. The nitro or tosyl compounds can be exchanged by nucleophilic aromatic substitution to generate ¹⁸F PET agents.

### Indolyl Amides of Indomethacin

The imaging agents in indolyl amide series utilized commercially available indomethacin which was transformed in 7 steps to either the fluoro-standard, Compound 389, or the PET precursor, Compound 390, using Scheme 1 depicted in FIG. 16. The development of this synthetic pathway was the result of several pathways tested. Indomethacin was first converted to the acetamide, Compound 301, followed by debenzoylation of the p-chlorobenzoyl group to give Compound 303. The 3^{rd} step involved the protection of the free amine using BOC anhydride so that selective benzylation of the indole nitrogen could be accomplished. Subsequent HCl_{(g)} deprotection of the BOC group followed by amidation using the appropriate p-substituted benzoic acid gave the PET precursor or fluorinated standard, Compounds 390 and 389, respectively, in good overall yield.

### Diamide Derivatives of Indomethacin

The synthesis of diamide indomethacin imaging agents required the selective amidation of only one of the two available amino groups present in the diamine tether. Dimer prevention was accomplished by the use of the mono tert-butoxycarbonyl (BOC) protected diamine. Treatment of indomethacin with mono BOC-ethylenediamine in the presence of ethyl-1-[3-(dimethylamino)propyl]-3-ethylcarbodiamide (EDCl) afforded the desired products in good yield using Scheme 2 (see FIG. 17). 1-hydroxybenzotriazole hydrate (HOBt) was employed, as it perturbed the generation of the stable, undesired N-acylurea byproduct. Deprotection of the BOC group was cleanly and efficiently accomplished by bubbling HCl gas through a solution of methylene chloride and the amino amide. Generation of the benzamide derivatives Compounds 354, 355, and 380-382, was accomplished with EDCl coupling in the presence of HOBt and DIPEA in DMF.

### Amide Derivatives of Indomethacin

The amide series can be synthesized from many routes, depending on the availability of starting materials. Preparation of the amide Compound 385 was accomplished by convenient HCl_{(g)} debenzoylation of Compound 360 to give Compound 375 followed by benzoylation using the corresponding acid chloride according to Scheme 3 (see FIG. 18).

Alternatively, Compound 375 was prepared from the commercially available indole acetic acid via EDCl coupling. o-Nitro benzaldehydes have been shown to undergo PET exchange (see Ekaeva et al., 1995), so the exchangeable group was placed ortho to the amide withdrawing group on the benzoyl chloride functionality. The 4-chloro-2-nitro benzylchloride (Compound 384) was prepared by stirring the benzoic acid starting material with thionyl chloride in DMF initially at room temperature until all HCl generation subsided followed by reflux for one hour. Benzoylation of Compound 384 to the indole nitrogen was accomplished by treatment of the indole with NaH for 10 minutes before Compound 384 was added.

In some embodiments, disclosed herein are reverse amides of indomethacin. The reverse amide series is different from those of the indomethacin series due to the placement of the amide bond. This amide “reversal” design was created to address the metabolic and hydrolytic instability associated with the conventional indomethacin analogs. Furthermore, amide bond hydrolysis in these compounds following in vivo administration in preclinical species will not generate indomethacin.

The diamide series was developed to address the feasibility of tethering bulky functional groups onto indomethacin to create a “dual function” inhibitor. The use of a long aliphatic chain allows the indomethacin functionality to fully insert into the binding pocket of COX-2 while the bulky secondary amide functional group resided in the more spacious lobby of the enzyme. Incorporating the diamine tether between indomethacin and p-fluorobenzamide aided this interaction. Extensive testing of Compound 355 has shown that this compound is selective and potent against COX-2 in free enzyme as well as intact cells.

Lastly, the amide series was developed in order to place the exchangeable group in the indomethacin core. This allows a large array of amides or esters to be prepared to address the issues of selectivity, potency, and half-life. The synthesis of a large series of derivatives could be accomplished by first benzoylating 5-methoxy-2-methyl indole with the appropriate PET sensitive acid chloride followed by amidation using a variety of amines.

An improved synthesis of the reverse amide intermediate has been accomplished to afford efficient reduction of the amine and selective benzylation at the indole nitrogen to give the key intermediate in gram scale quantities. The diamide series has been fully utilized for PET with the discovery that Compound 355 is a potent and specific inhibitor of COX-2 both in free enzyme as well as intact cells. The amide series also shows promise.

Tables 1-3 show several series of potential PET precursors as well as the ¹⁹F standards. Also provided are IC₅₀ values for certain of the derivatives for COX-1 and COX-2.

TABLE 1
Diamide Series Indomethacin Derivatives

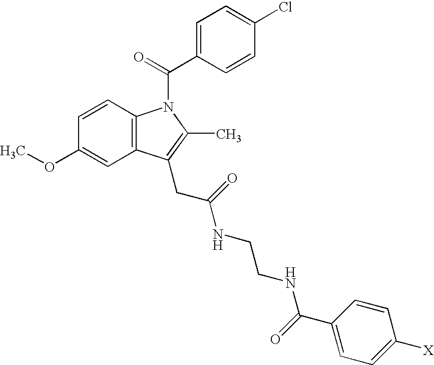

| Compound No. | X |
|---|---|
| 355 | F |
| 361 | NMe₃⁺ |
| 381 | I |
| 382 | NO₂ |
| 387 | OTs |

355: COX-1 IC₅₀ > 60 μM; COX-2 IC₅₀ 103 nM

TABLE 2
Reverse Amide Series Indomethacin Derivatives

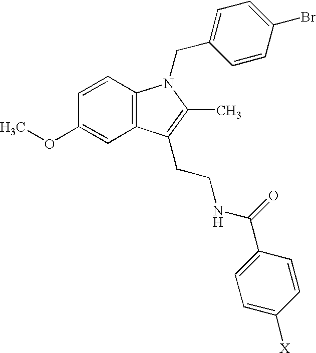

| Compound No. | X |
|---|---|
| 389 | F |
| 390 | NO₂ |
| — | I |
| — | OTs |
| — | NMe₃⁺ |

389: COX-1 IC₅₀ > 60 μM; COX-2 IC₅₀ 53 nM

TABLE 3
Amide Series Indomethacin Derivatives

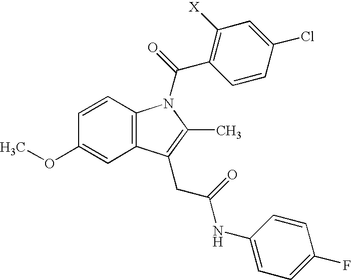

| Compound No. | X | R |
|---|---|---|
| 360 | F | NH—C₆H₄—F |
| 385 | NO₂ | NH—C₆H₄—F |
| 391 | F | OH |
| — | I | NH—C₆H₄—F |
| — | OTs | NH—C₆H₄—F |
| — | NMe₃⁺ | NH—C₆H₄—F |

360: COX-1 IC₅₀ > 60 μM; COX-2 IC₅₀ 100 nM

### Example 13

### Pharmacokinetics and Metabolism

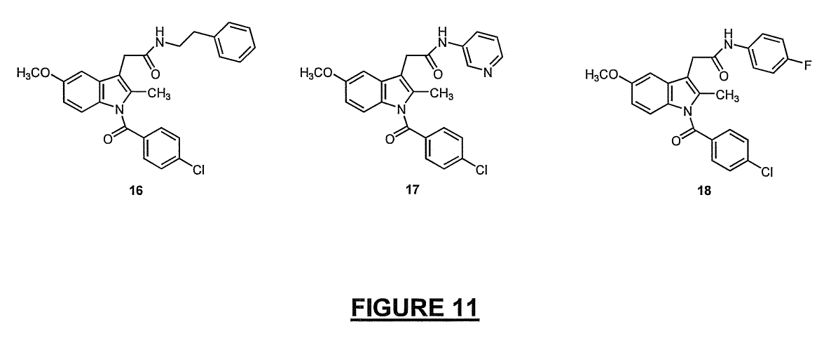

The in vivo pharmacokinetics and pharmacodynamics of the indomethacin derivatives are of interest in the design of an imaging agent, in that compounds that exhibit lengthy half-lives are more likely to reach target tissues. Detailed metabolic studies have been performed on three compounds, shown in FIG. 13. All three compounds are highly potent and selective COX-2 inhibitors, as indicated by IC₅₀ values for the purified enzyme of 0.060 μM, 0.060 μM, and 0.052 μM for Compounds 16, 17, and 18 (FIG. 11), respectively. All three compounds demonstrated IC₅₀ values for COX-1 of >66 μM.

Preliminary metabolic studies were conducted using isolated liver microsome preparations. Compound 16 was rapidly metabolized by rat, human, and mouse liver microsomes (0.125 mg/mL protein), with half-lives of 11 minutes, 21 minutes, and 51 minutes, respectively. Four metabolites were identified that arise by hydroxylation of the ethylene side chain and demethylation of the 5-methoxy group on the indole ring. The latter is a minor pathway of metabolism. Studies using specific inhibitors of cytochrome P450 isoforms, and purified recombinant enzymes demonstrated that side chain hydroxylation is catalyzed by CYP3A4, and O-demethylation is catalyzed by CYP2D6. No hydrolysis to indomethacin was observed in these studies, or during incubations of Compound 16 with rat liver cytosol or rat plasma. The finding that most of the metabolism of Compound 16 occurs in the amide side chain suggests that the use of more sterically hindered or electron-withdrawing substituents might improve compound stability. This was confirmed in the cases of Compounds 17 and 18, both of which were metabolized more slowly than Compound 16 by rat liver microsomes, (half-lives of 75 minutes, and 100 minutes, respectively).

Consistent with the data obtained with rat liver microsomes, Compound 16 demonstrated poor bioavailability, a short half-life, and a low maximal plasma concentration after oral dosing in rats, although a long terminal half-life was observed after intravenous dosing. In addition to the metabolites expected from the in vitro studies, indomethacin was detected in the plasma of treated rats. Approximately 4% of the administered dose was converted to indomethacin.

As predicted from its slower rate of microsomal metabolism, Compound 18 proved to be the most promising of the three compounds from a metabolic perspective. It exhibits 30% oral bioavailability, a clearance half-life of 4 hours, and a very low conversion to indomethacin in vivo (˜0.5% of the administered dose).

### Example 14

### In Vivo Anti-Inflammatory Efficacy

Despite their vast differences in pharmacokinetic parameters, both Compounds 16 and 18 are effective anti-inflammatory compounds in the rat carageenan footpad model. ED₅₀ values for Compounds 16 and 18 (0.8 mg/kg and 0.25 mg/kg, respectively) indicated favorable potency for these compounds as compared to indomethacin (ED₅₀=2 mg/kg). Although anti-inflammatory efficacy is not required for an imaging agent, the fact that these compounds have comparable or superior potency to indomethacin confirms that they reach and bind to COX-2 in vivo, a desirable characteristic.

### Example 15

### Evaluation of Monochromatic X-Ray Imaging Agents

Compounds containing multiple iodine atoms can be used for monochromatic X-ray imaging. For the evaluation of these compounds, tumor-bearing and control mice are imaged with the monochromatic X-ray beam in a CT geometry both below and above the iodine K-edge. A cylindrical water bolus surrounds the mice to help attenuate the X-ray beam and to normalize exposure. The procedure is then repeated following intravenous administration of the imaging agent. The CT study is interpreted by a “blinded” radiologist to determine visibility of the tumors and any alteration in attenuation engendered by the administration of the COX-2 agent.

### Example 16

### Evaluation of PET Imaging Agents

For imaging, the COX-2 selective imaging agent is labeled with 0.5-1 mCi of a positron emitting agent: ¹⁸F. Test animals are sedated, placed in the micro-PET system, and then imaged in dynamic 3D mode following injection. Injection volume is small (0.1-0.3 ml). Dynamic images are acquired every 5 minutes for the first hour and then serial static images are performed each 30 minutes for 3 hours. Static images are approximately 15 minutes in duration, depending upon the actual injected activity level. Time-activity curves are generated for both normal and tumor regions and standard uptake ratio values are determined in order to quantify the degree of tumor enhancement.

### Example 17

### Evaluation of MRI Imaging Agents

MR imaging is performed either with a 4 cm volume coil for whole-body imaging or with a 2.5 cm (inner diameter) surface coil for implanted tumors. In all studies, the animals are imaged prior to and following the injection of the gadolinium-labeled COX-2 selective imaging agent. After injection, images are made sequentially. Images are acquired approximately every minute for 30 minutes and then every 15 minutes for a total period of 4 hours. Initially animals will be re-imaged at 24 hours. Images are analyzed using the U.S. National Institutes of Health (NIH) supplied image-analysis software package, ImageJ. Image signal-enhancement over both normal and tumor regions is quantified as both a function of time and dose level.

### Example 18

### Evaluation of COX-2-Selective Imaging Agents In Vivo

### HCA-7 Human Colon Carcinoma Xenografts

Imaging agents that target the COX-2 enzyme in vivo can be used to detect tumors expressing elevated levels of this enzyme. Agents that have been identified as promising using the described methods are tested in vivo using a number of tumor models. An exemplary model system is the HCA-7 human colon adenocarcinoma cell line. HCA-7 cells are readily cultured in vitro, and can be evaluated as tumor xenografts in vivo. They express COX-2, and it is well-documented that NSAIDs and selective COX-2 inhibitors cause a reduction in the size and number of colonies formed by these cells when grown in soft agar or matrigel. Similarly, NSAIDs and COX-2 inhibitors cause a reduction in tumor formation and growth of HCA-7 cell xenografts in nude mice (Sheng et al., 1997; Williams et al., 2000b; Mann et al., 2001).

Tumor xenografts are established by injecting 10⁶ HCA-7 cells suspended in 0.2 mL of culture medium into the dorsal subcutaneous tissue of athymic nude mice. Measurable solid tumors are detected within 1 to 2 weeks, at which point they are suitable for imaging studies. This model is particularly useful, because tumors form quickly in a well-defined, subcutaneous location, allowing testing of all imaging modalities under nearly ideal conditions. Xenografts of HCT-116 cells, a colon cancer cell line that is not COX-2 dependent, are used as a negative control Sheng et al., 1997). The HCT-116 xenografts are also used to evaluate the level of COX-2 expression in tissue surrounding the tumor, a factor that has been shown to contribute to tumor angiogenesis and growth (Williams et al., 2000a).

### Example 19

### Evaluation of COX-2-Selective Imaging Agents In Vivo

### Murine Lewis Lung Carcinoma

Compounds that show promise in the HCA-7 xenograft model are tested against the murine Lewis lung carcinoma cell line. This cell line provides a syngeneic tumor model that can be used in C57BL/6 mice without concern of tumor rejection. Lewis lung carcinoma cells have been shown to express COX-2 in vitro and in vivo, and the administration of NSAIDs or COX-2 inhibitors has been shown to reduce cell proliferation and viability in vitro, and to reduce tumorigenesis and tumor growth in vivo (Stolina et al., 2000; Eli et al., 2001). Intravenous injection of Lewis lung carcinoma cells (5×10⁵) leads to the formation of lung tumors within 30 to 40 days. Subcutaneous injection of the cells (5×10⁵) leads to the formation of localized solid tumors. Therefore, as in the case of the HCA-7 xenograft, this model allows the testing of well-defined subcutaneous tumors, but also provides the opportunity to evaluate compounds for imaging tumors at the more challenging intrathoracic location.

### Example 20

### Evaluation of COX-2-Selective Imaging Agents In Vivo

### Murine Models of Colorectal Carcinoma

The HCA-7 and Lewis lung carcinoma models are advantageous, in that they allow the study of an imaging agent in a defined, solid tumor at a known location. However, ultimate clinical application will require the detection of small, spontaneous tumors that arise in situ. Two models of colon carcinogenesis are available that will allow the evaluation of imaging agents under these circumstances, the APC^{Min-} mouse model, and the azoxymethane tumorigenesis model.

### APC^{Min-} Mouse Model

Familial adenomatous polyposis (FAP) in humans is associated with the development of large numbers of intestinal adenomas at an early age, with progression to carcinomas over time. This condition results from mutation in the APC (adenomatous polyposis coli) gene, and a number of mouse models exist in which this gene has been altered, either by chemical exposure or by site-directed mutagenesis. The APC^{Min-} (multiple intestinal neoplasia) mouse model was developed through a chemically-induced germline mutation at codon 850 of the APC gene (Su et al., 1992; Moser et al., 1995). These mice develop multiple intestinal and colonic adenomas by 100 days of age. Increased expression of COX-2 has been demonstrated in the adenomas and surrounding stroma, and administration of NSAIDs and selective COX-2 inhibitors reduces both the number and size of adenoma formation (Boolbol et al., 1996; Williams et al., 1996; Barnes and Lee, 1998; Jacoby et al., 2000). In a similar model, APC^{Δ716}, coexpression of the APC mutation with targeted deletion of the COX-2 gene resulted in a reduced number and size of adenomas when compared to expression of the APC mutation in mice normozygous for COX-2 (Oshima et al., 1996).

### Azoxymethane-Induced Colon Carcinoma

A second well-defined model of colon tumorigenesis in rodents is derived from the subcutaneous injection of azoxymethane in weanling rats or mice. In this model, azoxymethane is administered subcutaneously or intraperitoneally at weekly doses of 10 to 15 mg/kg for a period of 2 to 6 weeks. Fully developed adenocarcinomas are observed at 30 to 50 weeks after treatment. Experiments in rats have demonstrated increased expression of COX-2 in azoxymethane-induced colonic tumors when compared to normal colonic tissue DuBois et al., 1996; Jacoby et al., 2000; Takahashi et al., 2000; Kishimoto et al., 2002a). Furthermore, NSAIDs and COX-2 inhibitors have been shown to decrease both the number and size of colonic tumors resulting from azoxymethane treatment in both rats and mice (Yoshima et al., 1997; Fukutake et al., 1998; Reddy et al., 2000; Kishimoto et al., 2002b). In order to generate tumors for use in assessing the utility of imaging agents, 6 week old male mice will be treated for 6 weeks with weekly intraperitoneal injections of 10 mg/kg azoxymethane (Fukutake et al. 1998).

Both the APC^{Min-} mouse model and the mouse azoxymethane-induced colon carcinoma model are used to determine the effectiveness of promising imaging agents. The azoxymethane model poses the disadvantage that over 7 months are required for tumor formation. However, because the tumors generated in this model are highly COX-2 dependent, and because the prior research in this model is extensive, this model is a valuable system in which to evaluate compounds. In both models, imaging agents are assessed at various points during disease progression in order to determine the effectiveness of each agent to detect tumors at early stages. Results are correlated with pathological evaluation of intestinal tissue.

### REFERENCES

The references listed below as well as all references cited in the specification are incorporated herein by reference to the extent that they supplement, explain, provide a background for, or teach methodology, techniques, and/or compositions employed herein.
Alexander V. (1995) Design and Synthesis of Macrocyclic Ligands and Their Complexes of Lanthanides and Actinides. Chem. Rev. 95, 273-342.
Allison, Howatson, Torrence, Lee, and Russell (1992) Gastrointestinal Damage Associated with the Use of Nonsteroidal Antiinflammatory Drugs. N. Engl. J. Med. 327, 749-754.
Barnes, C. J. and Lee, M. (1998) Chemoprevention of spontaneous intestinal adenomas in the adenomatous polyposis coli Min mouse model with aspirin. Gastroenterology 114, 873-877.
Block, D., Coenen, H. H. and Stocklin, G. (1988) ¹⁸F-Fluoroacylation via fluorocarboxylic acid esters. J. Labelled Compds. Radiopharm. 25:185-200.
Boolbol, S. K., Dannenberg, A. J., Chadburn, A., Martucci, C., Guo, X. J., Ramonefti, J. T., Abreu-Goris, M., Newmark, H. L., Lipkin, M. L., DeCosse, J. J. and Bertagnolli, M. M. (1996) Cyclooxygenase-2 overexpression and tumor formation are blocked by sulindac in a murine model of familial adenomatous polyposis. Cancer Res. 56, 2556-2560.
Buskens, C. J., Van Rees, B. P., Sivula, A., Reitsma, J. B., Haglund, C., Bosma, P. J., Offerhaus, G. J., Van Lanschot, J. J. and Ristimaki, A. (2002) Prognostic significance of elevated cyclooxygenase 2 expression in patients with adenocarcinoma of the esophagus. Gastroenterology 122, 1800-1807.
Caesar J. et al. (1961) The use of Indocyanine green in the measurement of hepatic blood flow and as a test of hepatic function. Clin. Sci. 21, 43-57.
Chulada, P. C., Thompson, M. B., Mahler, J. F., Doyle, C. M., Gaul, B. W., Lee, C., Tiano, H. F., Morham, S. G., Smithies, O. and Langenbach, R. (2000) Genetic disruption of Ptgs-1, as well as Ptgs-2, reduces intestinal tumorigenesis in Min mice. Cancer Res. 60, 4705-4708.
Daniel, T. O., Liu, H., Morrow, J. D., Crews, B. C. and Marnett, L. J. (1999) Thromboxane A2 is a mediator of cyclooxygenase-2-dependent endothelial migration and angiogenesis. Cancer Res. 59, 4574-4577.
Denkert, C., Kobel, M., Berger, S., Siegert, A., Leclere, A., Trefzer, U. and Hauptmann, S. (2001) Expression of cyclooxygenase 2 in human malignant melanoma. Cancer Res. 61, 303-308.
DeWitt, D. L. and Smith, W. L. (1988) Primary Structure of Prostaglandin G/H Synthase from Sheep Vesicular Gland Determined from the Complementary DNA Sequence. Proc. Natl. Acad. Sci. U.S.A. 85, 1412-1416.
DeWitt, D. L., El-Harith, E. A., Kraemer, S. A., Andrews, M. J., Yao, E. F., Armstrong, R. L. and Smith, W. L. (1990) The aspirin and heme-binding sites of ovine and murine prostaglandin endoperoxide synthases. J. Biol. Chem. 265, 5192-5198.
DuBois, R. N., Abramson, S. B., Crofford, L., Gupta, R. A., Simon, L. S., van de Putte, L. B. A. and Lipsky, P. E. (1998) Cyclooxygenase in biology and disease. FASEB J. 12, 1063-1073.
DuBois, R. N., Radhika, A., Reddy, B. S. and Entingh, A. J. (1996) Increased cyclooxygenase-2 levels in carcinogen-induced rat colonic tumors. Gastroenterology 110, 1259-1262.
Eberhart, C. E., Coffey, R. J., Radhika, A., Giardiello, F. M., Ferrenbach, S. and DuBois, R. N. (1994) Up-regulation of cyclooxygenase 2 gene expression in human colorectal adenomas and adenocarcinomas. Gastroenterology 107, 1183-1188.
Eli, Y., Przedecki, F., Levin, G., Kariv, N. and Raz, A. (2001) Comparative effects of indomethacin on cell proliferation and cell cycle progression in tumor cells grown in vitro and in vivo. Biochem. Pharmacol. 61, 565-571.
Ekaeva, I., et al. (1995) 2-[F-18]Fluorophenol and 4-[F-18]Fluorophenol from Baeyer-Villiger Oxidation of [F-18] Fluorophenylketones and [F-18]Fluorobenzaldehydes. Applied Radiation and Isotopes, 46, 777-782.
Fletcher, B. S., Kujubu, D. A., Perrin, D. M. and Herschman, H. R. (1992) Structure of the mitogen-inducible TIS10 gene and demonstration that the TIS10-encoded protein is a functional prostaglandin G/H synthase. J. Biol. Chem. 267, 4338-4344.
Fu, J.-Y., Masferrer, J. L., Seibert, K., Raz, A. and Needleman, P. (1990) The induction and suppression of prostaglandin H₂ synthase (cyclooxygenase) in human monocytes. J. Biol. Chem. 265, 16737-16740.
Fukutake, M., Nakatsugi, S., Isoi, T., Takahashi, M., Ohta, T., Mamiya, S., Taniguchi, Y., Sato, H., Fukuda, K., Sugimura, T. and Wakabayashi, K. (1998) Suppressive effects of nimesulide, a selective inhibitor of cyclooxygenase-2, on azoxymethane-induced colon carcinogenesis in mice. Carcinogenesis 19, 1939-1942.
He Y. L., Tanigami H., Ueyama H., Mashimo T., Yoshiya I. (1998) Measurement of blood volume using indocyanine green measured with pulse-spectrometry: Its reproducibility and reliability. Critical Care Medicine 26, 1446-1451.
Herschman, H. R. (1996) Prostaglandin synthase 2. Biochim. Biophys. Acta Lipids Lipid Metab. 1299, 125-140.
Hla, T. and Neilson, K. (1992) Human cyclooxygenase-2 cDNA. Proc. Natl. Acad. Sci. USA 89, 7384-7388.
Hochgesang, G. P. Jr., Nemeth-Cawley, J. F., Rowlinson, S. W., Caprioli, R. M. and Marnett, L. J. (in press) Functional analysis of the molecular determinants of cycloxygenase-2 acetylation by 2-acetoxyphenylhept-2-ynyl sulfide. Arch. Biochem. Biophys.
Hutchens, J. O., Wagner, H., Podolsky, B. and McMahon, T. M. (1949) Effect of ethanol and various metabolites on fluoroacetate poisoning. J. Pharmacol. Exptl. Therap. 95, 62-70.
Ishiwata, K., Ishii, S. I. and Senda, M. (1995) Successive preparation of ¹¹C labeled sodium acetate and/or sodium hexanoate. Appl. Radiat. Isot. 46, 1035-1037.
Jackels (1990) Pharm. Med. Imag, Section III, Chap. 20, p 645.
Jacoby, R. F., Seibert, K., Cole, C. E., Kelloff, G. and Lubet, R. A. (2000) The cyclooxygenase-2 inhibitor celecoxib is a potent preventive and therapeutic agent in the min mouse model of adenomatous polyposis. Cancer Res. 60, 5040-5044.
Jalilian, A. R., Afarideh, H., Najafi, R., Shafiee, A. and Bineshmarvasti, M. (2000) A new method of one-step, no carrier-added synthesis of cholesteryl 4-[¹⁸F]-fluorobenzoate ([¹⁸F]-CFB), a radiotracer in detection of adrenal malignancies. J. Pharm. Pharmaceut. Sci. 3, 114-124.
Kalgutkar, A. S., Crews, B. C., Rowlinson, S. W., Garner, C., Seibert, K. and Marnett, L. J. (1998a) Aspirin-like molecules that covalently inactivate cyclooxygenase-2. Science 280, 1268-1270.
Kalgutkar, A. S., Kozak, K. R., Crews, B. C., Hochgesang Jr., G. P. and Marnett, L. J. (1998b) Covalent modification of cyclooxygenase-2 (COX-2) by 2-(acetoxyphenyl)alkyl sulfides, a new class of selective COX-2 inactivators. J. Med. Chem. 41, 4800-4818.
Kalgutkar, A. S., Crews, B. C., Rowlinson, S. W., Marnett, A. B., Kozak, K. R., Remmel, R. P. and Marnett, L. J. (2000a) Biochemically based design of cyclooxygenase-2 (COX-2) inhibitors: facile conversion of nonsteroidal antiinflammatory drugs to potent and highly selective COX-2 inhibitors. Proc. Natl. Acad. Sci. USA 97, 925-930.
Kalgutkar, A. S., Marnett, A. B., Crews, B. C., Remmel, R. P. and Marnett, L. J. (2000b) Ester and amide derivatives of the nonsteroidal antiinflammatory drug, indomethacin, as selective cyclooxygenase-2 inhibitors. J. Med. Chem. 43, 2860-2870.
Kalgutkar, A. S., Rowlinson, S. W., Crews, B. C. and Marnett, L. J. (2002) Amide derivatives of meclofenamic acid as selective cyclooxygenase-2 inhibitors. Bioorg. Med. Chem. Lett. 12, 521-524.
Kandil, H. M., Tanner, G., Smalley, W., Halter, S., Radhika, A. and Dubois, R. N. (2001) Cyclooxygenase-2 expression in Barrett's esophagus. Dig. Dis. Sci. 46, 785-789.
Kargman, S. L., O'Neill, G. P., Vickers, P. J., Evans, J. F., Mancini, J. A. and Jothy, S. (1995) Expression of prostaglandin G/H synthase-1 and -2 protein in human colon cancer. Cancer Res. 55, 2556-2559.
Kawamori, T., Rao, C. V., Seibert, K. and Reddy, B. S. (1998) Chemopreventive activity of celecoxib, a specific cyclooxygenase-2 inhibitor, against colon carcinogenesis. Cancer Res. 58, 409-412.
Kishimoto, Y., Morisawa, T., Hosoda, A., Shiota, G., Kawasaki, H. and Hasegawa, J. (2002a) Molecular changes in the early stage of colon carcinogenesis in rats treated with azoxymethane. J. Exp. Clin. Cancer Res. 21, 203-211.
Kishimoto, Y., Yashima, K., Morisawa, T., Shiota, G., Kawasaki, H. and Hasegawa, J. (2002b) Effects of cyclooxygenase-2 inhibitor NS-398 on APC and c-myc expression in rat colon carcinogenesis induced by azoxymethane. J Gastroenterol. 37, 186-193.
Kozak, K. R., Rowlinson, S. W. and Marnett, L. J. (2000) Oxygenation of the Endocannabinoid, 2-Arachidonylglycerol, to Glyceryl Prostaglandins by Cyclooxygenase-2. J. Biol. Chem. 275, 33744-33749.
Kujubu, D. A., Fletcher, B. S., Varnum, B. C., Lim, R. W. and Herschman H. R. (1991) TIS10, A Phorbol Ester Tumor Promoter Inducible mRNA from Swiss 3T3 Cells, Encodes a Novel Prostaglandin Synthase/Cyclooxygenase Homologue. J. Biol. Chem. 266, 12866-12872.
Kurumbail, R. G., Stevens, A. M., Gierse, J. K., McDonald, J. J., Stegeman, R. A., Pak, J. Y., Gildehaus, D., Miyashiro, J. M., Penning, T. D., Seibert, K., Isakson, P. C. and Stallings, W. C. (1996) Structural basis for selective inhibition of cyclooxygenase-2 by anti-inflammatory agents. Nature 384, 644-648.
Lamanna, C. and Hart, E. R. (1968) Relationship of lethal toxic dose to body weight of the mouse. Toxicol. Appl. Pharmacol. 13, 307-315.
Langenbach, R., Morham, S. G., Tiano, H. F., Loftin, C. D., Ghanayem, B. I., Chulada, P. C., Mahler, J. F., Lee, C. A., Goulding, E. H., Kluckman, K. D., Kim, H. S, and Smithies, O. (1995) Prostaglandin synthase 1 gene disruption in mice reduces arachidonic acid-induced inflammation and indomethacin-induced gastric ulceration. Cell 83, 483-492.
Lanzo, C. A., Sutin, J., Rowlinson, S. W., Talley, J. and Marnett, L. J. (2000) Fluorescence quenching analysis of the association and dissociation of a diaryheterocycle to cyclooxygenase-1 and cyclooxygenase-2: The dynamic basis of cyclooxygenase-2 selectivity. Biochemistry 39, 6228-6234.
Lee S. H., Soyoola E., Chanmugam P., Hart S., Sun W., Zhong H., Liou S., Simmons D. and Hwang D. (1992) Selective Expression of Mitogen-Inducible Cyclooxygenase in Macrophages Stimulated with Lipopolysaccharide. J. Biol. Chem. 267, 25934-25938.
Luong, C., Miller, A., Barnett, J., Chow, J., Ramesha, C. and Browner, M. F. (1996) Flexibility of the NSAID binding site in the structure of human cyclooxygenase-2. Nature Struct Biol. 3, 927-933.
Mann, M., Sheng, H., Shao, J., Williams, C. S., Pisacane, P. I., Sliwkowski, M. X. and DuBois, R. N. (2001) Targeting cyclooxygenase 2 and HER-2/neu pathways inhibits colorectal carcinoma growth. Gastroenterology 120, 1713-1719.
Manning, H. C., Goebel, T., Marx, J. N. and Bornhop, D. J. (2002) Facile, efficient conjugation of a trifunctional lanthanide chelate to a peripheral benzodiazepine receptor ligand. Org. Lett. 4, 1075-1078.
Marnett, L. J., Siedlik, P, H, Ochs, R. C., Pagels, W. R., Das, M., Honn, K. V., Warnock, R. H., Tainer, B. E. and Eling, T. E. (1984) Mechanism of the stimulation of prostaglandin H synthase and prostacyclin synthase by the antithrombotic and antimetastatic agent, nafazatrom. Mol Pharmacol 26, 328-35.
Marnett, L. J. (1992) Aspirin and the potential role of prostaglandins in colon cancer. Cancer Res. 52, 5575-5589.
Marnett, L. J. and DuBois, R. N. (2002) COX-2: A Target for Colon Cancer Prevention. Annu. Rev. Pharmacol. Toxicol. 42, 55-80.
Masferrer, J. L., Leahy, K. M., Koki, A. T., Zweifel, B. S., Settle, S. L., Woerner, B. M., Edwards, D. A., Flickinger, A. G., Moore, R. J. and Seibert, K. (2000) Antiangiogenic and antitumor activities of cyclooxygenase-2 inhibitors. Cancer Res. 60, 1306-1311.
McCarthy, T. J., Sheriff, A. U., Graneto, M. J., Talley, J. J. and Welch, M. J. (2002) Radiosynthesis, in vitro validation, and in vivo evaluation of 18F-labeled COX-1 and COX-2 inhibitors. J. Nucl. Med. 43, 117-124.
Meade, E. A., Smith, W. L. and DeWitt, D. L. (1993) Differential inhibition of prostaglandin endoperoxide synthase (cyclooxygenase) isozymes by aspirin and other non-steroidal anti-inflammatory drugs. J. Biol. Chem. 268, 6610-6614.
Meyer et al. (1990) Invest. Radiol. 25, S53.
Morham, S. G., Lagenbach, R., Loftin, C. D., Tiano, H. F., Vouloumanos, N., Jennette, J. C., Mahler, J. F., Kluckman, K. D., Ledford, A., Lee, C. A. and Smithies, O. (1995) Prostaglandin synthase 2 gene disruption causes severe renal pathology in the mouse. Cell 83, 473-482.
Moser, A. R., Luongo, C., Gould, K. A., McNeley, M. K., Shoemaker, A. R. and Dove, W. F. (1995) ApcMin: a mouse model for intestinal and mammary tumorigenesis. Eur. J. Cancer 31A, 1061-1064.
Mujumdar R. B., Ernst L. A., Mujumdar S. R., Lewis C. J. and Waggoner A. S. (1993) Cyanine dye labeling reagents: Sulformdocyanine succinimidyl esters. Bioconjugate Chemistry 4, 105-111.
Odenwaller, R., Chen, Y. N. and Marnett, L. J. (1990) Preparation and proteolytic cleavage of apoprostaglandin endoperoxide synthase. Methods Enzymol 187, 479-85.
Ohki, S., Ogino, N., Yamamoto, S, and Hayaishi, O. (1979) Prostaglandin hydroperoxidase, an integral part of prostaglandin endoperoxide synthetase from bovine vesicular gland microsomes. J. Biol. Chem. 254, 829-836.
Oshima, M., Dinchuk, J. E., Kargman, S., Oshima, H., Hancock, B., Kwong, E., Trzaskos, J. M., Evans, J. F. and Taketo, M. M. (1996) Suppression of intestinal polyposis in Apc^{Δ716} knockout mice by inhibition of cyclooxygenase 2 (COX-2). Cell 87, 803-809.
O'Sullivan, M. G., Huggins, E. M. Jr., and McCall C. E. (1993) Lipopolysaccharide-Induced Expression of Prostaglandin H Synthase-2 in Alveolar Macrophages is Inhibited by Dexamethasone by not by Aspirin. Biochem. Biophys. Res. Commun. 191, 1294-1300.
PCT International Publication WO 96/17628
PCT International Publication WO 98/22146
PCT International Publication WO 98/48838
PCT International Publication WO 98/48846
Peters, R. A. (1952) Lethal synthesis. Proc. R. Soc. Lond. B. 139, 143-170.
Peters, R. A., Wakelin, R. W. and Buffa, P. (1953) Biochemistry of fluoroacetate poisoning: Isolation and some properties of the fluorotricarboxylic acid inhibitor of citrate metabolism. Proc. Roy. Soc. (London) B140, 497-506.
Picot, D., Loll, P. J. and Garavito, R. M. (1994) The X-ray crystal structure of the membrane protein prostaglandin H₂ synthase-1. Nature 367, 243-249.
Pomper M. G. and Port J. D. (2000) New techniques in MR imaging of brain tumors. Magn Reson Imaging Clin N Am 8, 691-713.
Reddy, B. S., Hirose, Y., Lubet, R., Steele, V., Kelloff, G., Paulson, S., Seibert, K. and Rao, C. V. (2000) Chemoprevention of colon cancer by specific cyclooxygenase-2 inhibitor, celecoxib, administered during different stages of carcinogenesis. Cancer Res. 60, 293-297.
Ristimaki, A., Nieminen, O., Saukkonen, K., Hotakainen, K., Nordling, S. and Haglund, C. (2001) Expression of cyclooxygenase-2 in human transitional cell carcinoma of the urinary bladder. Am. J. Pathol. 158, 849-853.
Ristimaki, A., Sivula, A., Lundin, J., Lundin, M., Salminen, T., Haglund, C., Joensuu, H. and Isola, J. (2002) Prognostic Significance of Elevated Cyclooxygenase-2 Expression in Breast Cancer. Cancer Res. 62, 632-635.
Sheng, H., Shao, J., Kirkland, S. C., Isakson, P., Coffey, R. J., Morrow, J. D., Beauchamp, R. D. and DuBois, R. N. (1997) Inhibition of human colon cancer cell growth by selective inhibition of cyclooxygenase-2. J. Clin. Invest. 99, 2254-2259.
Sheng, H., Shao, J., Washington, M. K. and DuBois, R. N. (2001) Prostaglandin E2 increases growth and motility of colorectal carcinoma cells. J. Biol. Chem. 276, 18075-18081.
Smith, C. J., Morrow, J. D., Roberts, L. J. I. and Marnett, L. J. (1993) Differentiation of monocytoid THP-1 cells with phorbol ester induces expression of prostaglandin endoperoxide synthase-1 (COX-1). Biochem. Biophys. Res. Commun. 192, 787-793.
Smith, W. L., DeWitt, D. L. and Garavito, R. M. (2000) Cyclooxygenases: structural, cellular, and molecular biology. Annu. Rev. Biochem. 69, 145-182.
Smith, W. L., Garavito, R. M. and DeWitt, D. L. (1996) Prostaglandin endoperoxide H synthases (cyclooxygenases)-1 and -2. J. Biol. Chem. 271, 33157-33160.
Soslow, R. A., Dannenberg, A. J., Rush, D., Woerner, B. M., Khan, K. N., Masferrer, J. and Koki, A. T. (2000) COX-2 is expressed in human pulmonary, colonic, and mammary tumors. Cancer 89, 2637-2645.
Steinbach, G., Lynch, P. M., Phillips, R. K., Wallace, M. H., Hawk, E., Gordon, G. B., Wakabayashi, N., Saunders, B., Shen, Y., Fujimura, T., Su, L. K. and Levin, B. (2000) The effect of celecoxib, a cyclooxygenase-2 inhibitor, in familial adenomatous polyposis. N. Engl. J. Med. 342, 1946-1952.
Stolina, M., Sharma, S., Lin, Y., Dohadwala, M., Gardner, B., Luo, J., Zhu, L., Kronenberg, M., Miller, P. W., Portanova, J., Lee, J. C. and Dubineft, S. M. (2000) Specific inhibition of cyclooxygenase 2 restores antitumor reactivity by altering the balance of IL-10 and IL-12 synthesis. J. Immunol. 164, 361-370.
Su, L.-K., Kinzler, K. W., Vogelstein, B., Preisinger, A. C., Moser, A. R., Luongo, C., Gould, K. A. and Dove, W. F. (1992) Multiple intestinal neoplasia caused by a mutation in the murine homolog of the APC gene. Science 256, 668-670.
Tada, M., Oikawa, A., Iwata, R., Sato, K., Kubota, K., Fujiwara, T., Sugiyama, H., Abe, Y., Sato, T., Matsuzaa, T., Takahashi, H., Wakui, A. and Ido, T. (1990) A rapid and efficient synthesis of 2-deoxy-2-[18F]fluoro-acetamido-D-mannopyranose and -D-galactopyranose. J. Labelled Compd. Radiopharm. 23, 847-853.
Takahashi, M., Mutoh, M., Kawamori, T., Sugimura, T. and Wakabayashi, K. (2000) Altered expression of beta-catenin, inducible nitric oxide synthase and cyclooxygenase-2 in azoxymethane-induced rat colon carcinogenesis. Carcinogenesis 21, 1319-1327.
Timofeevski, S. L., Prusakiewicz, J. J., Rouzer, C. A. and Marnett, L. J. (2002) Isoform-selective interaction of cyclooxygenase-2 with indomethacin amides studied by real-time fluorescence, inhibition kinetics, and site-directed mutagenesis. Biochemistry 41, 9654-9662.
Tsujii, M. and DuBois, R. N. (1995) Alterations in cellular adhesion and apoptosis in epithelial cells overexpressing prostaglandin endoperoxide synthase 2. Cell 83, 493-501.
Tucker, O. N., Dannenberg, A. J., Yang, F. K., Zhang, F., Teng, L. S., Daly, J. M., Soslow, R. A., Masferrer, J. L., Woerner, B. M., Koki, A. T. and Fahey, T. J. I. (1999) Cyclooxygenase-2 expression is up-regulated in human pancreatic cancer. Cancer Res. 59, 987-990.
U.S. Pat. Nos. 4,885,363; 5,087,440; 5,155,215; 5,188,816; 5,219,553; 5,262,532; 5,358,704; 5,453,505; 5,865,754; 5,928,627; 6,083,486; 6,207,700; 6,246,901; 6,306,890; 6,399,647; 6,403,625.
van den Hoff, J., Burchert, W., Borner, A. R., Fricke, H., Kuhnel, G., Meyer, G. J., Otto, D., Weckesser, E., Wolpers, H. G. and Knapp, W. H. (2001) [1-(11)C]Acetate as a quantitative perfusion tracer in myocardial PET. J. Nucl. Med. 42, 1174-1182.
Van Der Ouderaa, F. J., Buytenhek, M., Nugteren, D. H. and Van Dorp, D. A. (1980) Acetylation of prostaglandin endoperoxide synthetase with acetylsalicylic acid. Eur. J. Biochem. 109, 1-8.
Vane, J. R. and Botting, R. M. (1996) Mechanism of action of anti-inflammatory drugs. Scand. J. Rheumatol. 25(Suppl. 102), 9-21.
Vane, J. R., Bakhle, Y. S. and Botting, R. M. (1998) Cyclooxygenases 1 and 2. Annu. Rev. Pharmacol. Toxicol. 38, 97-120.
Ward, J. C. and Spencer, D. A. (1947) Notes on the pharmacology of sodium fluoroacetate: Compound 1080. J. Am. Pharm. Assoc. 36, 59-62.
Weissleder, R. (2001) A clearer vision for in vivo imaging. Nat. Biotechnol. 19, 316-317.
Weissleder R., Tung C. H., Mahmood U., and Bogdanov A. Jr. (1999) In vivo imaging of tumors with protease-activated near-infrared fluorescent probes. Nat Biotechnol 17, 375-378.
Williams, C. S., Luongo, C., Radhika, A., Zhang, T., Lamps, L. W., Nanney, L. B., Beauchamp, R. D. and DuBois, R. N. (1996) Elevated cyclooxygenase-2 levels in Min mouse adenomas. Gastroenterology 111, 1134-1140.
Williams, C. S., Tsujii, M., Reese, J., Dey, S. K. and DuBois, R. N. (2000a) Host cyclooxygenase-2 modulates carcinoma growth. J. Clin. Invest. 105, 1589-1594.
Williams, C. S., Watson, A. J., Sheng, H., Helou, R., Shao, J. and DuBois, R. N. (2000b) Celecoxib prevents tumor growth in vivo without toxicity to normal gut: lack of correlation between in vitro and in vivo models. Cancer Res. 60, 6045-6051.
Wilson, K. T., Fu, S., Ramanujam, K. S. and Meltzer, S. J. (1998) Increased expression of inducible nitric oxide synthase and cyclooxygenase-2 in Barrett's esophagus and associated adenocarcinomas. Cancer Res. 58, 2929-2934.
Wilson (1991) Optical properties of tissues. Encyclopedia of Human Biology, 5, 587-597.
Xie, W., Chipman, J. G., Robertson, D. L., Erikson, R. L. and Simmons, D. L. (1991) Expression of a mitogen-responsive gene encoding prostaglandin synthase is regulated by mRNA splicing. Proc. Natl. Acad. Sci. USA 88, 2692-2696.
Yoshimi, N., Kawabata, K., Hara, A., Matsunaga, K., Yamada, Y. and Mori, H. (1997) Inhibitory effect of NS-398, a selective cyclooxygenase-2 inhibitor, on azoxymethane-induced aberrant crypt foci in colon carcinogenesis of F344 rats. Jpn. J. Cancer Res. 88, 1044-1051.
Yokoyama, C. and Tanabe, T. (1989) Cloning of Human Gene Encoding Prostaglandin Endoperoxide Synthase and Primary Structure of the Enzyme. Biochem. Biophys. Res. Commun. 165, 888-894.

It will be understood that various details of the described subject matter can be changed without departing from the scope of the described subject matter. Furthermore, the foregoing description is for the purpose of illustration only, and not for the purpose of limitation.

What is claimed is:

1. A radiological imaging agent comprising a detectable group and a COX-2-selective ligand, wherein the COX-2-selective ligand is a derivative of a non-steroidal anti-inflammatory drug (NSAID) comprising an ester moiety or a secondary amide moiety.

2. The radiological imaging agent of claim 1, wherein a carboxyl group of the non-steroidal anti-inflammatory drug has been derivatized to an ester or secondary amide.

3. The radiological imaging agent of claim 1, wherein the radiological imaging agent comprises the following structure: 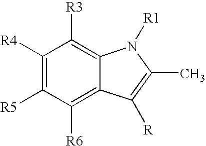
and further wherein:
R is selected from the group consisting of 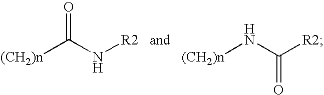
R1 is selected from the group consisting of a detectable group, 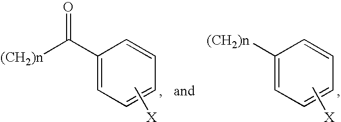
wherein X is a halogen or a radioactive isotope thereof at one or more positions of the aromatic ring;
R2 comprises a detectable group or a halo substituted aryl;
R3-R6 are each independently selected from the group consisting of hydrogen; halo; C₁ to C₆ alkyl or branched alkyl; C₁ to C₆ alkoxy or branched alkoxy; benzyloxy; SCH₃; SOCH₃; SO₂CH₃; SO₂NH₂; and CONH₂;
n is 0-5 inclusive;
and further at least one of R1 and R2 comprises a detectable group.

4. The radiological imaging agent of claim 3, wherein the radiological imaging agent comprises the following structure: 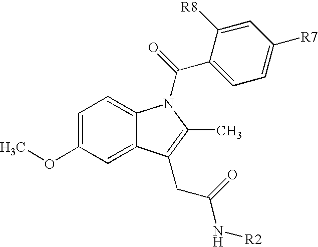
and further wherein:
R7 comprises a halogen;
R8 is selected from the group consisting of hydrogen, a halogen, C₁-C₆ alkyl or branched alkyl, and C₁-C₆ aryl or branched aryl; and
R2 comprises a detectable group.

5. The radiological imaging agent of claim 4, wherein:
R2 is selected from the group consisting of 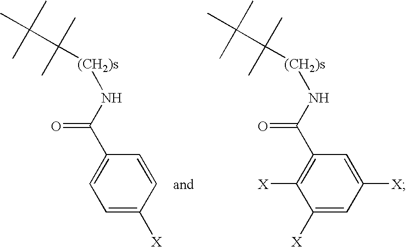
and
X is a halogen selected from the group consisting of fluorine, iodine, and radioactive isotopes thereof.

6. The radiological imaging agent of claim 5, wherein at least one of the halogens present in the R2 group is selected from the group consisting of ¹⁸F, ¹²³I, and ¹²⁵I.

7. The radiological imaging agent of claim 6, wherein R2 is 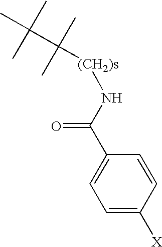
and X is ¹⁸F.

8. The radiological imaging agent of claim 4, wherein:
R7 is Cl;
R2 has the following structure: 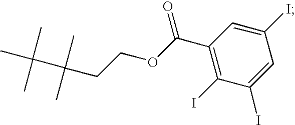
and at least one iodine atom is selected from the group consisting of ¹²³I and ¹²⁵I.

9. The radiological imaging agent of claim 4, wherein:
R7 is Cl;
R2 has the following structure: 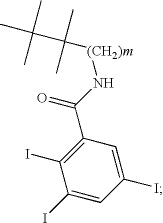
m=an integer between 0 and 8, inclusive; and
at least one iodine atom is selected from the group consisting of ¹²³I and ¹²⁵I.

10. The radiological imaging agent of claim 4, wherein R7 is Cl and R2 has the following structure: 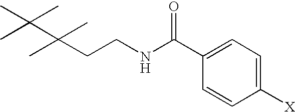
and further wherein X is a halogen or a radioactive isotope thereof.

11. The radiological imaging agent of claim 10, wherein X is ¹⁸F.

12. The radiological imaging agent of claim 4, wherein R7 is Cl and R2 has the following structure:

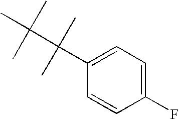

13. The radiological imaging agent of claim 3, wherein:
the radiological imaging agent comprising the following structure: 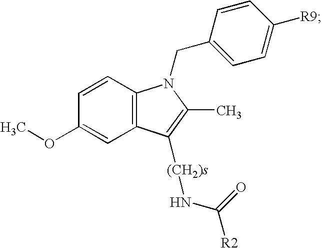
R1 is a halogen;
R2 is p-halobenzene;
and s is an integer from 1 to 4 inclusive.

14. The radiological imaging agent of claim 13, wherein R9 is Br, n=2, and R2 is p-¹⁸F-benzene.

15. A radiological imaging agent comprising a detectable group and an indomethacin derivative selected from the group consisting of:

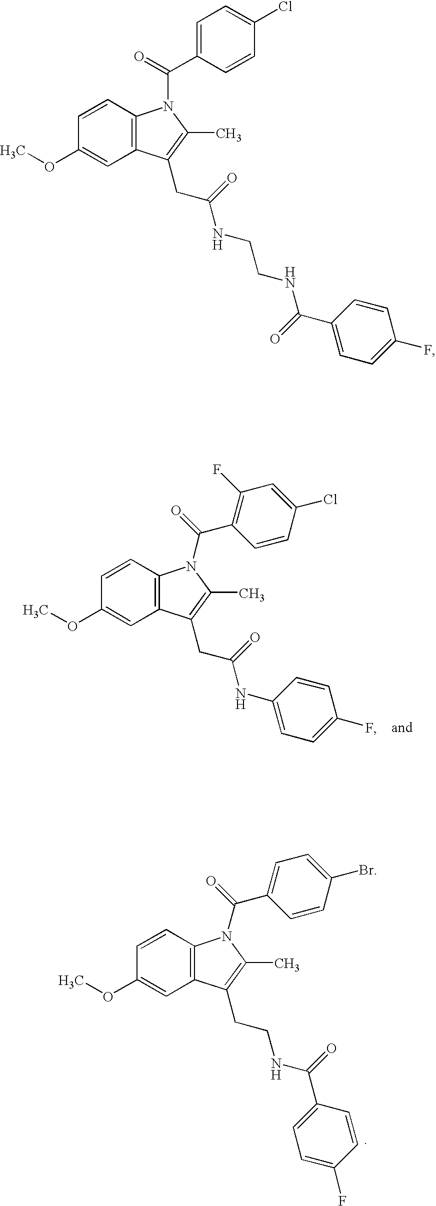

16. The radiological imaging agent of claim 15, wherein one or more fluorine atoms present is ¹⁸F.

17. A radiological imaging agent comprising the following structure: 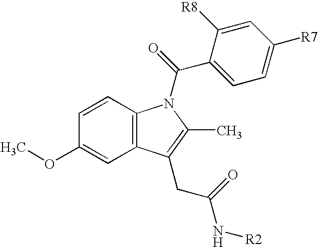
wherein:
R2 is selected from the group consisting of 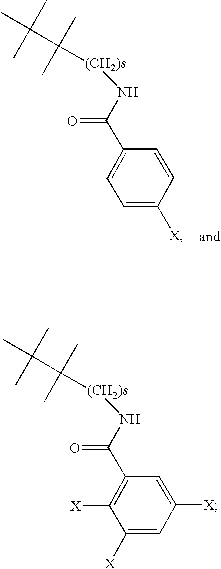
R7 is a halogen;
R8 is selected from the group consisting of hydrogen, a halogen, C₁-C₆ alkyl, C₁-C₆ branched alkyl, C₁-C₆ aryl, and C₁-C₆ branched aryl;
s is an integer from 1 to 4 inclusive; and
X is a halogen selected from the group consisting of fluorine, iodine, and radioactive isotopes thereof.

18. The radiological imaging agent of claim 17, wherein at least one of the halogens present in the R2 group is selected from the group consisting of ¹⁸F, ¹²³I, and ¹²⁵I.
